SEQUENCE LISTING (8 sequences)

SEQ ID NO: 1 - DNA, 1940 bp, Homo sapiens
gcgggactgtagaggcgcctataagggaagttgttcagtcaactcggaaaaagggtaacaacccggaaagtagactcaccgtcttggtctagagactgacccctgcacagacagaccccttcccctctctgcgaaaggaccaagccccagaagtcactccatctcctacggctcgcaatttccagaggccccctggcaccttccagcctgatgtcgtgcgtcaagttatggcccagcggtgcccccgcccccttggtgtccatcgaggaactggagaaccaggagctcgtcggcaaaggcgggttcggcacagtgttccgggcgcaacataggaagtggggctacgatgtggcggtcaagatcgtaaactcgaaggcgatatccagggaggtcaaggccatggcaagtctggataacgaattcgtgctgcgcctagaaggggttatcgagaaggtgaactgggaccaagatcccaagccggctctggtgactaaattcatggagaacggctccttgtcggggctgctgcagtcccagtgccctcggccctggccgctcctttgccgcctgctgaaagaagtggtgcttgggatgttttacctgcacgaccagaacccggtgctcctgcaccgggacctcaagccatccaacgtcctgctggacccagagctgcacgtcaagctggcagattttggcctgtccacatttcagggaggctcacagtcagggacagggtccggggagccagggggcaccctgggctacttggccccagaactgtttgttaacgtaaaccggaaggcctccacagccagtgacgtctacagcttcgggatcctaatgtgggcagtgcttgctggaagagaagttgagttgccaaccgaaccatcactcgtgtacgaagcagtgtgcaacaggcagaaccggccttcattggctgagctgccccaagccgggcctgagactcccggcttagaaggactgaaggagctaatgcagctctgctggagcagtgagcccaaggacagaccctccttccaggaatgcctaccaaaaactgatgaagtcttccagatggtggagaacaatatgaatgctgctgtctccacggtaaaggatttcctgtctcagctcaggagcagcaataggagattttctatcccagagtcaggccaaggagggacagaaatggatggctttaggagaaccatagaaaaccagcactctcgtaatgatgtcatggtttctgagtggctaaacaaactgaatctagaggagcctcccagctctgttcctaaaaaatgcccgagccttaccaagaggagcagggcacaagaggagcaggttccacaagcctggacagcaggcacatcttcagattcgatggcccaacctccccagactccagagacctcaactttcagaaaccagatgcccagccctacctcaactggaacaccaagtcctggaccccgagggaatcagggggctgagagacaaggcatgaactggtcctgcaggaccccggagccaaatccagtaacagggcgaccgctcgttaacatatacaactgctctggggtgcaagttggagacaacaactacttgactatgcaacagacaactgccttgcccacatggggcttggcaccttcgggcaaggggaggggcttgcagcaccccccaccagtaggttcgcaagaaggccctaaagatcctgaagcctggagcaggccacagggttggtataatcatagcgggaaataaagcaccttccaagcttgcctccaagagttacgagttaaggaagagtgccaccccttgaggcccctgacttccttctagggcagtctggcctgcccacaaactgactttgtgacctgtcccccaggagtcaataaacatgatggaatgctagtcaaaaaaaaaaaaaaaaaaaa

SEQ ID NO: 2 - protein, 518 aa, Homo sapiens
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNCSGVQVGDNNYLTMQQTTALPTWGLAPSGKGRGLQHPPPVGSQEGPKDPEAWSRPQGWYNHSGK

SEQ ID NO: 3 - DNA, 4165 bp, Homo sapiens
gaacgcgcgcgcctccccggctagtcccggcaggcggagccccagttagactgctcgtcaagtgtgggaaaagctccgtggcgtcacaagctactatataaaaggcggtgcccgccggggccgagtgggagtccgcggcgagcgcagcagcagggcccggtcctgcgcctcgggagtcggcgtccaggctcggagcgcgacacggagactaggtggcagggtacagctctgccggggggggaaaaagtggtaccattttgggcgttcttgagcttcagaatgcaaccagacatgtccttgaatgtcattaagatgaaatccagtgacttcctggagagtgcagaactggacagcggaggctttgggaaggtgtctctgtgtttccacagaacccagggactcatgatcatgaaaacagtgtacaaggggcccaactgcattgagcacaacgaggccctcttggaggaggcgaagatgatgaacagactgagacacagccgggtggtgaagctcctgggcgtcatcatagaggaagggaagtactccctggtgatggagtacatggagaagggcaacctgatgcacgtgctgaaagccgagatgagtactccgctttctgtaaaaggaaggataattttggaaatcattgaaggaatgtgctacttacatggaaaaggcgtgatacacaaggacctgaagcctgaaaatatccttgttgataatgacttccacattaagatcgcagacctcggccttgcctcctttaagatgtggagcaaactgaataatgaagagcacaatgagctgagggaagtggacggcaccgctaagaagaatggcggcaccctctactacatggcgcccgagcacctgaatgacgtcaacgcaaagcccacagagaagtcggatgtgtacagctttgctgtagtactctgggcgatatttgcaaataaggagccatatgaaaatgctatctgtgagcagcagttgataatgtgcataaaatctgggaacaggccagatgtggatgacatcactgagtactgcccaagagaaattatcagtctcatgaagctctgctgggaagcgaatccggaagctcggccgacatttcctggcattgaagaaaaatttaggcctttttatttaagtcaattagaagaaagtgtagaagaggacgtgaagagtttaaagaaagagtattcaaacgaaaatgcagttgtgaagagaatgcagtctcttcaacttgattgtgtggcagtaccttcaagccggtcaaattcagccacagaacagcctggttcactgcacagttcccagggacttgggatgggtcctgtggaggagtcctggtttgctccttccctggagcacccacaagaagagaatgagcccagcctgcagagtaaactccaagacgaagccaactaccatctttatggcagccgcatggacaggcagacgaaacagcagcccagacagaatgtggcttacaacagagaggaggaaaggagacgcagggtctcccatgacccttttgcacagcaaagaccttacgagaattttcagaatacagagggaaaaggcactgcttattccagtgcagccagtcatggtaatgcagtgcaccagccctcagggctcaccagccaacctcaagtactgtatcagaacaatggattatatagctcacatggctttggaacaagaccactggatccaggaacagcaggtcccagagtttggtacaggccaattccaagtcatatgcctagtctgcataatatcccagtgcctgagaccaactatctaggaaatacacccaccatgccattcagctccttgccaccaacagatgaatctataaaatataccatatacaatagtactggcattcagattggagcctacaattatatggagattggtgggacgagttcatcactactagacagcacaaatacgaacttcaaagaagagccagctgctaagtaccaagctatctttgataataccactagtctgacggataaacacctggacccaatcagggaaaatctgggaaagcactggaaaaactgtgcccgtaaactgggcttcacacagtctcagattgatgaaattgaccatgactatgagcgagatggactgaaagaaaaggtttaccagatgctccaaaagtgggtgatgagggaaggcataaagggagccacggtggggaagctggcccaggcgctccaccagtgttccaggatcgaccttctgagcagcttgatttacgtcagccagaactaaccctggatgggctacggcagctgaagtggacgcctcacttagtggataaccccagaaagttggctgcctcagagcattcagaattctgtcctcactgataggggttctgtgtctgcagaaattttgtttcctgtacttcatagctggagaatggggaaagaaatctgcagcaaaggggtctcactctgttgccaggctggtctcaaacttctggactcaagtgatcctcccgcctcggccttccaaagtgctgggatatcaggcactgagccactgcgcccagccaacaatccgctctgaggaaagcgtaagcaggaagacctcttaatggcatagcaccaataaaaaaatgactcctagttgtgtttggaaagggagagaagagatgtctgaggaaggtcatgttctttcagcttatggcatttcctagagttttgttgaagcaagaagaaaaactcagagaatataaaatcaacttttaaaattgtgtgctctcttcttcacgtaggctcctgttaaaaacaaagtgcagtcagattctaagccctgttcagagacttcgtggatcacagctgcagctcaccgccacatcacaggatccgttaacgttaatacccaatactctgtcagccactgtaggctctaagaaccacgtgcagtcttcagcccattaaattatcgattattttttaatgaattgaatttatattgagtcttcaaattaactgaatggatttaaaggggtaccaaggaggggggaaacatcagaatttcccaggcagttgttgcaaggaattggtactaaccgtgactacaacaaaaattcttgattgacttttaaagttatttcctggcattctggtaccttcacccagcctgagtgccctggagagggaacaggaaatgctgatctctacccctgggtgagaccagaacctcagggctgatactgttgagtggcttcctcggtttactctgtgtactgtgaaagtattttcatattttttctgtgtgccagagtgaaaaaggacagcttctgagtgtggtaattgtgcctctagcacccagcctttcaaagcccacctgaaacctgggggtggatgaaagaactagaatagaagactgaagctgggtaggccgctcagtgtccactggcattttgctaaaccgacaaggaaggctgtgtgcttagctctccccagagggagggcgagaagggtgtggtgatggtcaatctggctgtcggaacagattctggtgtcttgggctgataacagtgttgttgattctgattgtgaatcccctcaactctagcagacacatacacacccctgaaatggggctgcagagcaggctgtctcagccttgccactgtcggcatctcggcctgggtaattctgttgtggggactgtcctgttccttgtaggatgtttagtagcatccctgcccccacctactagatgccaggggcactgttctccccagccccccgccccagttgtgacaatagtctctaaacattgtcaaatggtccaaggaaaggggaaaattgccccggttgagaagagcactgctgtaaagtaatgagcctcggctctcctgtctgcacctgtccggttactacttggccaccacgcagccttggctcctacagcccaaaagggagaatggagggaggctccaggctttgctggaggggcctgggtgagttctgtttgctccttgtaccaccatccaaatggtgttatcaaatctcttagattccaaagaggttgaataattaatgttcaaaggcaagagggcaaggcattttttaacactttttaaaataaaaatttataccacaactttaaaaaaaaaaaaaaaaaa

SEQ ID NO: 4 - protein, 671 aa, Homo sapiens
MQPDMSLNVIKMKSSDFLESAELDSGGFGKVSLCFHRTQGLMIMKTVYKGPNCIEHNEALLEEAKMMNRLRHSRVVKLLGVIIEEGKYSLVMEYMEKGNLMHVLKAEMSTPLSVKGRIILEIIEGMCYLHGKGVIHKDLKPENILVDNDFHIKIADLGLASFKMWSKLNNEEHNELREVDGTAKKNGGTLYYMAPEHLNDVNAKPTEKSDVYSFAVVLWAIFANKEPYENAICEQQLIMCIKSGNRPDVDDITEYCPREIISLMKLCWEANPEARPTFPGIEEKFRPFYLSQLEESVEEDVKSLKKEYSNENAVVKRMQSLQLDCVAVPSSRSNSATEQPGSLHSSQGLGMGPVEESWFAPSLEHPQEENEPSLQSKLQDEANYHLYGSRMDRQTKQQPRQNVAYNREEERRRRVSHDPFAQQRPYENFQNTEGKGTAYSSAASHGNAVHQPSGLTSQPQVLYQNNGLYSSHGFGTRPLDPGTAGPRVWYRPIPSHMPSLHNIPVPETNYLGNTPTMPFSSLPPTDESIKYTIYNSTGIQIGAYNYMEIGGTSSSLLDSTNTNFKEEPAAKYQAIFDNTTSLTDKHLDPIRENLGKHWKNCARKLGFTQSQIDEIDHDYERDGLKEKVYQMLQKWVMREGIKGATVGKLAQALHQCSRIDLLSSLIYVSQN

SEQ ID NO: 5 - DNA, 1317 bp, Homo sapiens
tgagatcacttcccttgcagactttggaagggagagcactttattacagaccttggaagcaagaggattgcattcagcctagttcctggttgctggccaaagggatcatggacattgaagcatattttgaaagaattggctataagaactctaggaacaaattggacttggaaacattaactgacattcttgagcaccagatccgggctgttccctttgagaaccttaacatgcattgtgggcaagccatggagttgggcttagaggctatttttgatcacattgtaagaagaaaccggggtgggtggtgtctccaggtcaatcaacttctgtactgggctctgaccacaatcggttttcagaccacaatgttaggagggtatttttacatccctccagttaacaaatacagcactggcatggttcaccttctcctgcaggtgaccattgacggcaggaattacattgtcgatgctgggtctggaagctcctcccagatgtggcagcctctagaattaatttctgggaaggatcagcctcaggtgccttgcattttctgcttgacagaagagagaggaatctggtacctggaccaaatcaggagagagcagtatattacaaacaaagaatttcttaattctcatctcctgccaaagaagaaacaccaaaaaatatacttatttacgcttgaacctcgaacaattgaagattttgagtctatgaatacatacctgcagacgtctccaacatcttcatttataaccacatcattttgttccttgcagaccccagaaggggtttactgtttggtgggcttcatcctcacctatagaaaattcaattataaagacaatacagatctggtcgagtttaaaactctcactgaggaagaggttgaagaagtgctgagaaatatatttaagatttccttggggagaaatctcgtgcccaaacctggtgatggatcccttactatttagaataaggaacaaaataaacccttgtgtatgtatcacccaactcactaattatcaacttatgtgctatcagatatcctctctaccctcacgttattttgaagaaaatcctaaacatcaaatactttcatccataaaaatgtcagcatttattaaaaaacaataactttttaaagaaacataaggacacattttcaaattaataaaaataaaggcattttaaggatggcctgtgattatcttgggaagcagagtgattcatgctagaaaacatttaatattgatttattgttgaattcatagtaaatttttactggtaaatgaataaagaatattgtgg

SEQ ID NO: 6 - protein, 290 aa, Homo sapiens
MDIEAYFERIGYKNSRNKLDLETLTDILEHQIRAVPFENLNMHCGQAMELGLEAIFDHIVRRNRGGWCLQVNQLLYWALTTIGFQTTMLGGYFYIPPVNKYSTGMVHLLLQVTIDGRNYIVDAGSGSSSQMWQPLELISGKDQPQVPCIFCLTEERGIWYLDQIRREQYITNKEFLNSHLLPKKKHQKIYLFTLEPRTIEDFESMNTYLQTSPTSSFITTSFCSLQTPEGVYCLVGFILTYRKFNYKDNTDLVEFKTLTEEEVEEVLRNIFKISLGRNLVPKPGDGSLTI

SEQ ID NO: 7 - DNA, 4729 bp, Homo sapiens
ctttggaaagtcccgtggaaatccccgggcctacaacccgcatacaactgaaacggggcaaagcagactgcgcagtctgcagtcttcgtggcgggccaagcgagcttggagcccgcgggggcggagcggtgagagcggccgccaagagagatcacacccccagccgaccctgccagcgagcgagcccgaccccaggcgtccatggagcgtcgcctccgcccggtccctgccccgacccccgcctgcggcgcgctcctgccttgaccaggacttgggactttgcgaaaggatcgcggggcccggagaggtaaccgccgcgcctcccggagaggtgttggagagcacaatggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggctaaccggaaacaggtgggtcacctcctgcaagaagtggggcctcgagctgtcagtcatcatggtgctatcctctgaacccctcagctgccactgcaacagtgggcttaagggtgtctgagcaggagaggaaagataagctcttcgtggtgcccacgatgctcaggtttggtaacccgggagtgttcccaggtggccttagaaagcaaagcttgtaactggcaagggatgatgtcagattcagcccaaggttcctcctctcctaccaagcaggaggccaggaacttctttggacttggaaggtgtgcggggactggccgaggcccctgcaccctgcgcatcaggactgcttcatcgtcttggctgagaaagggaaaagacacacaagtcgcgtgggttggagaagccagagccattccacctcccctcccccagcatctctcagagatgtgaagccagatcctcatggcagcgaggccctctgcaagaagctcaaggaagctcagggaaaatggacgtattcagagagtgtttgtagttcatggtttttccctacctgcccggttcctttcctgaggacccggcagaaatgcagaaccatccatggactgtgattctgaggctgctgagactgaacatgttcacattgacagaaaaacaagctgctctttataatatgcaccttttaaaaaattagaatattttactgggaagacgtgtaactctttgggttattactgtctttacttctaaagaagttagcttgaactgaggagtaaaagtgtgtacatatataatatacccttacattatgtatgagggatttttttaaattatattgaaatgctgccctagaagtacaataggaaggctaaataataataacctgttttctggttgttgttggggcatgagcttgtgtatacactgcttgcataaactcaaccagctgcctttttaaagggagctctagtcctttttgtgtaattcactttatttattttattacaaacttcaagattatttaagtgaagatatttcttcagctctggggaaaatgccacagtgttctcctgagagaacatccttgctttgagtcaggctgtgggcaagttcctgaccacagggagtaaattggcctctttgatacacttttgcttgcctccccaggaaagaaggaattgcatccaaggtatacatacatattcatcgatgtttcgtgcttctccttatgaaactccagctatgtaataaaaaactatactctgtgttctgttaatgcctctgagtgtcctacctccttggagatgagatagggaaggagcagggatgagactggcaatggtcacagggaaagatgtggccttttgtgatggttttattttctgttaacactgtgtcctgggggggctgggaagtcccctgcatcccatggtaccctggtattgggacagcaaaagccagtaaccatgagtatgaggaaatctctttctgttgctggcttacagtttctctgtgtgctttgtggttgctgtcatatttgctctagaagaaaaaaaaaaaaggaggggaaatgcattttccccagagataaaggctgccattttgggggtctgtacttatggcctgaaaatatttgtgatccataactctacacagcctttactcatactattaggcacactttccccttagagccccctaagtttttcccagacgaatctttataatttctttccaaagataccaaataaacttcagtgttttcatctaattctcttaaagttgatatcttaatattttgtgttgatcattatttccattcttaatgtgaaaaaaagtaattatttatacttattataaaaagtatttgaaatttgcacatttaattgtccctaatagaaagccacctattctttgttggatttcttcaagtttttctaaataaatgtaacttttcacaagagtcaacattaaaaaataaattatttaagaacagaaaaaaaaaaaaaaaa

SEQ ID NO: 8 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

(19) United States
(12) Patent Application Publication
(10) Pub. No.: US 2021/0147842 A1
(43) Pub. Date: May 20, 2021

(54) METHODS AND COMPOSITIONS FOR INHIBITING NECROPTOSIS IN NEUROVASCULAR AND/OR NEURODEGENERATIVE DISEASES OR DISORDERS

(71) Applicant: President and Fellows of Harvard College, Cambridge, MA (US)

(72) Inventors: Junying YUAN, Waban, MA (US); Chengyu Zou, So. Weymouth, MA (US); Lauren Mifflin, Boston, MA (US)

(21) Appl. No.: 17/097,695

(22) Filed: Nov. 13, 2020

Related U.S. Application Data
(60) Provisional application No. 62/934,899, filed on Nov. 13, 2019.

(51) Int. Cl. C12N 15/113; A61P 25/28

(52) U.S. Cl. CPC C12N 15/113; A61P 25/28; C12Y 203/01005; C12N 2310/122; C12N 2750/14143

(57) ABSTRACT

The present disclosure provides agents, compositions, and methods for inhibiting necroptosis in the brains of subjects in need thereof. In some embodiments, agents, compositions, and methods provided herein are useful for treating necroptosis-mediated neurovascular or neurodegenerative diseases or disorders, e.g., Alzheimer's disease.

### RELATED APPLICATIONS

This application is claims the benefit of priority to U.S. Provisional Patent Application Ser. No. 62/934,899, filed Nov. 13, 2019, which is hereby incorporated herein by reference in its entirety.

### BACKGROUND

In many diseases, cell death is mediated through apoptotic and/or necrotic pathways. While much is known about the mechanisms of action that control apoptosis, control of necrosis is not as well understood. Understanding the mechanisms regulating both necrosis and apoptosis in cells is essential to being able to treat conditions, such as neurovascular diseases, neurodegenerative diseases, stroke, coronary heart disease, kidney disease, and liver disease.

Cell death has traditionally been categorized as either apoptotic or necrotic based on morphological characteristics (Wyllie et al., Int. Rev. Cytol. 30 68: 251 (1980)). These two modes of cell death were also initially thought to occur via regulated (caspase-dependent) and non-regulated processes, respectively. More recent studies, however, demonstrate that the underlying cell death mechanisms resulting in these two phenotypes are much more complicated and, under some circumstances, interrelated. Furthermore, conditions that lead to necrosis can occur by either regulated caspase-independent or non-regulated processes.

One regulated caspase-independent cell death pathway with morphological features resembling necrosis, called necroptosis, has recently been described (Degterev et al., Nat. Chem. Biol. 1: 112 (2005)). This manner of cell death can be initiated with various stimuli (e.g., TNF-α and Fas ligand) and in an array of cell types (e.g., monocytes, fibroblasts, lymphocytes, macrophages, epithelial cells and neurons). Necroptosis may represent a significant contributor to and, in some cases, predominant mode of cellular demise under pathological conditions involving excessive cell stress, rapid energy loss, and massive oxidative species generation, where the highly energy-dependent apoptosis process is not operative.

There is a need to prevent, treat, stabilize, or lessen the severity or progression of diseases or disorders associated with necroptosis, e.g., necroptosis-mediated neurovascular or neurodegenerative diseases or disorders such as Alzheimer's disease.

### SUMMARY

The present disclosure provides technologies relating to inhibiting necroptosis in the brains of subjects in need thereof, including agents and compositions that inhibit necroptosis, e.g., necroptosis induced by tumor necrosis factor alpha (TNFα), and methods of using the same. The present disclosure also provides methods of treating neurovascular diseases or disorders associated with endothelial cell necroptosis, including, e.g., Alzheimer's disease (AD).

The present disclosure recognizes, among other things, that endothelial cell necroptosis in brain can contribute to development of a neurovascular or neurodegenerative disease or disorder, e.g., AD. Thus, a neurovascular or neurodegenerative disease or disorder, e.g., AD, can be treated or prevented by modulating expression and/or activity of one or more molecular mediators involved in a necroptosis pathway in the brain.

Accordingly, one aspect provided herein relates to a method of treating a neurovascular or neurodegenerative disease or disorder in a subject. Such a method comprises inhibiting or decreasing necroptosis in brain endothelial cells of a subject suffering from or susceptible to a neurovascular or neurodegenerative disease or disorder. An exemplary neurovascular or neurodegenerative disease or disorder is Alzheimer's disease.

In some embodiments, a step of inhibiting or decreasing necroptosis in brain endothelial cells of a subject in need thereof comprises performing at least one of the following steps:
a. inhibiting or decreasing expression and/or activity of receptor-interacting serine/threonine-protein kinase 3 (RIPK3) in the brain endothelial cells of the subject;
b. inhibiting or decreasing expression and/or activity of receptor-interacting serine/threonine-protein kinase 1 (RIPK1) in the brain endothelial cells of the subject;
c. increasing expression and/or activity of N-acetyltransferase 1 (Nat1) or N-acetyltransferase 2 (Nat2) in the brain endothelial cells of the subject;
d. increasing expression and/or activity of tumor necrosis factor alpha-induced protein 3 (Tnfaip3/A20) in the brain endothelial cells of the subject; and
e. increasing expression and/or activity of low density lipoprotein receptor-related protein 1 (LRP1) in the brain endothelial cells of the subject.

In some embodiments involving a method described herein, a step of inhibiting or decreasing necroptosis comprises a step of inhibiting or decreasing expression and/or activity of RIPK3 in the brain endothelial cells of a subject in need thereof; and the step of inhibiting or decreasing expression and/or activity of RIPK3 comprises administering to the subject a brain endothelial-cell-specific viral vector that expresses a nucleic acid agent that inhibits expression of RIPK3. In some embodiments, a nucleic acid agent delivered by a brain-endothelial-cell-specific viral vector to inhibit or decrease expression and/or activity of RIPK3 is or comprises an RNA agent whose nucleotide sequence is sufficiently complementary to that of an RIPK3 transcript, or portion thereof, that the nucleic acid agent hybridizes to the RIPK3 transcript. For example, when the nucleic acid agent is present, RIPK3 level, RIPK3 transcript level, or both, is reduced as compared with otherwise comparable conditions when the nucleic acid agent is absent. In some embodiments, a nucleic acid agent delivered by a brain-endothelial-cell-specific viral vector (e.g., an adeno-associated viral vector) to inhibit or decrease expression and/or activity of RIPK3 is or comprises a short hairpin RNA (shRNA).

In some embodiments involving a method described herein, a step of inhibiting or decreasing necroptosis comprises a step of increasing expression and/or activity of N-acetyltransferase 1 (Nat1) or N-acetyltransferase 2 (Nat2) in the brain endothelial cells of a subject in need thereof; and the step of increasing expression and/or activity of Nat1 or Nat2 comprises administering to the subject a brain endothelial-cell-specific viral vector (e.g., an adeno-associated viral vector) comprising a nucleic acid agent that encodes Nat1 or Nat2, or a functional fragment thereof. In some embodiments, when such a nucleic acid agent is present, Nat1 or Nat2 level, Nat1 or Nat2 transcript level, or both is increased as compared with otherwise comparable conditions when the nucleic acid agent is absent. In some embodiments, when a subject to be treated is a mouse subject, a step of inhibiting necroptosis comprises a step of increasing expression of Nat1. In some embodiments, when a subject to be treated is a human subject, a step of inhibiting necroptosis comprises a step of increasing expression of Nat2.

In some embodiments involving a method described herein, necroptosis is inhibited in brain endothelial cells, which comprise or are cerebral endothelial cells, blood-brain barrier endothelial cells, brain microvessels or microvasculature, or combinations thereof.

In some embodiments involving a method described herein, a subject in need thereof is suffering from or susceptible to neuroinflammation in the subject's brain; and a step of inhibiting or decreasing necroptosis in the brain endothelial cells of the subject comprises inhibiting or decreasing to an extent sufficient that the neuroinflammation is reduced, e.g., as assessed by detecting level of interferon or interleukin-1β or both.

The present disclosure also recognizes that Nat1 or Nat2 is an upstream regulator of a necrosis signaling pathway. Accordingly, a method of inhibiting necroptosis in the brain of a subject in need thereof comprising increasing expression and/or activity of Nat1 or Nat2 in the brain of the subject is provided herein. When a subject in need of necroptosis inhibition is a mouse subject, such a method comprises administering an agonist of Nat1 to the subject. When a subject in need of necroptosis inhibition is a human subject, such a method comprises administering an agonist of Nat2 to the subject.

In some embodiments involving a method described herein, a step of increasing expression and/or activity of Nat1 or Nat2 comprises increasing to an extent sufficient that receptor-interacting serine/threonine-protein kinase 1 (RIPK1) activation in the brain of a subject being treated is inhibited. In some embodiments involving a method described herein, a step of increasing expression and/or activity of Nat1 or Nat2 comprises increasing to an extent sufficient that level of tumor necrosis factor alpha-induced protein 3 (A20) in the brain of a subject being treated is increased. In some embodiments involving a method described herein, a step of increasing expression and/or activity of Nat1 or Nat2 comprises increasing to an extent sufficient that level of low density lipoprotein receptor-related protein 1 (LRP1) in the brain of a subject being treated is increased.

In some embodiments involving a method described herein, wherein an agonist of Nat1 or Nat2 administered to a subject in need thereof is or comprises an expression vector comprising a nucleic acid sequence that encodes Nat1 or Nat2, or a functional fragment thereof. An expression vector may comprise a cerebral endothelial-cell-specific viral vector (e.g., adeno-associated viral vector). In some embodiments, when such an agonist is present, Nat1 or Nat2 level, Nat1 or Nat2 transcript level, or both is increased as compared with otherwise comparable conditions when the agonist is absent.

In some embodiments involving a method described herein, a subject in need thereof is suffering from or susceptible to a necroptosis-mediated neurovascular disease or disorder (e.g., Alzheimer's disease).

These, and other aspects encompassed by the present disclosure, are described in more detail below and in the claims.

### BRIEF DESCRIPTION OF THE DRAWINGS

FIG. 1: Single-cell RNA sequencing of brain endothelial cells. Panel a shows the experimental workflow. APPswePSEN1dE9 (APP/PS1) mice and its littermate controls (WT) were sacrificed at the age of 5-6 months to obtain single-cell suspensions from brain, which were then sorted into CD31^{high} and CD45^{low} ECs. Brain ECs were encapsulated with hydrogel beads in the inDrops microfluidic device for single-cell RNA-seq. t-SNE plot of the 4538 single cells were separated into 10 clusters. Panel b illustrates endothelial arteriovenous zonation. Three endothelial clusters (CD31+) with 3686 cells were identified as venous endothelial cell (vEC), capillary endothelial cell (capEC) and arterial endothelial cell (aEC) with known arteriovenous markers. Panel c illustrates the number of different expression genes between WT and APP/PS1 groups detected by SCDE in vEC, capEC and aEC clusters. Panel d shows a GO gene set analysis of genes detected in Panel c.

FIG. 2A-D: The loss of vECs and capECs in APP/PS1 mice. FIG. 2A shows proportions of endothelial cells along the venous to arterial axis in WT and APP/PS1 mice.

FIGS. 2B and 2C illustrate brain endothelial cells in the cerebral cortex marked with anti-CD31 antibody through immunochemistry. aECs were identified with Acta2 staining, while Vwf were used as a marker for both aECs and vECs. Imaris was used for three-dimensional reconstruction of microvessels and compartmentalization of aECs (Acta2⁺), vECs (Acta2⁻Vwf⁺) and capECs(Vwf⁻). FIG. 2D shows quantifications of arterial, venous and capillary microvessels reveal the loss of vECs and capECs in APP/PS1 mice.

FIG. 3A-G: RIPK1 is observed to mediate brain endothelial dysfunctions in Alzheimer's disease. FIG. 3A shows that the gene expression level of RIPK1 decreases along the venous to arterial endothelial axis. Gkn3 serves a marker of aEC. FIG. 3B shows published endothelial single-cell sequencing data (Vanlandewijck, M. et al. Nature 554, 475-580 (2018)) also confirms the decrease of RIPK1 gene expression along venous-arterial zonation. FIG. 3C demonstrates that RIPK1 is activated in microvessels of Alzheimer's brain. Microvessels from mouse brains and human brains were isolated as described in Munikoti et al. Journal of neuroscience methods 207: 80-85 (2012) and Boulay et al. JoVE, e53208 (2015), the contents of each of which are incorporated herein by reference in their entireties for the purposes stated herein. p-S166 RIPK1 was immunoprecipitated and then detected by anti-RIPK1 antibody. FIG. 3D shows that activated RIPK1 is detected in endothelial cells of Alzheimer's brain. Sections of cerebral cortex from WT, APP/PS1 and APP/PS1;RIPK1-138N mouse brain were immunostained for DAPI, CD31 and p-S166 RIPK1 (pRIPK1). White arrow heads indicate endothelial cells that are positive for pRIPK1 staining and the scale bar is 20 μm. FIG. 3E demonstrates that genetic inhibition of RIPK1 activation prevents brain endothelial loss in the cerebral cortex of APP/PS1 mice. Brain endothelial cells were identified with CD31 staining and quantified by Imaris. The scale bar is 20 μm. FIG. 3F shows that RIPK1 inhibition rescues blood brain barrier (BBB) leakage in APP/PS1 mice. 10 kD-tetramethyrhodamine-labeled dextran tracer was injected into the circulation of mice. White arrow head points to the leaked dextran and the scale bar is 50 μm. FIG. 3G demonstrates that the migration of neutrophils into the cerebral parenchyma is prevented with the genetic inhibition of RIPK1. Neutrophils were stained with anti-Gr-1 antibody and pointed by the white arrowhead. The scale bar is 20 μm.

FIG. 4A-G: Specific knockdown of RIPK3 in brain endothelial cells rescues microvascular and cognitive dysfunctions in APP/PS1 mice. FIG. 4A demonstrates that the protein expression of RIPK3 is decreased in cerebral microvasculature endothelial cells via the injection of adeno-associated virus (AAV) expressing RIPK3 shRNA. APP/PS1 mice were intravenously injected with brain microvascular endothelial cell specific AAV (1×10¹¹ genomic particles per mouse) that expresses either scrambled shRNA (Ctrl-AAV) or RIPK3 shRNA (RIPK3-shRNA-AAV) at the age of 4 months. After 6 weeks, mice were sacrificed. Cerebral cortex sections were stained with anti-RIPK3 and anti-CD31 antibody. The scale bar is 25 um. FIGS. 4B, 4C and 4D show that RIPK3 knockdown (RIPK3-KD) in brain endothelial cells rescues the loss of brain microvessels, BBB leakage and neutrophil penetration into cerebral parenchyma of APP/PS1 mice. The scale bar represents 40, 50 and 20 μm. FIG. 4E demonstrates that inflammatory cytokines, interferon-gamma (IFN) and interleukin 1 (3 (IL-1(3), in the brain of APP/PS1 mice are reduced with specific knockdown of RIPK3 in endothelial cells. FIGS. 4F and 4G show that specific RIPK3-KD in brain microvascular endothelial cells prevents cognitive decline of APP/PS1 mice. Novel object recognition test (FIG. 4F) and water T maze test (FIG. 4G) both revealed RIPK3-KD in brain endothelial cells has rescued cognitive behaviors in APP/PS1 mice.

FIG. 5: The gene expression levels of Caspase 3 (Casp3), Caspase 8 (Casp8), Caspase 9 (Cap9), RIPK3, Mlk1 and TNF receptor (Tnfrsfla) along venous to arterial endothelial axis obtained from the published endothelial single cell sequencing data (Vanlandewijck, M. et al. Nature 554, 475-580 (2018)).

FIGS. 6A-B: TNF and Aβ oligomers induce RIPK1 and RIPK3 dependent necroptosis in primary brain endothelial cells. Primary brain endothelial cells from adult mice (WT, RIPK1-D138N or RIPK3-KO mice) were cultured as described in Welser-Alves, J. V., et al. Methods in molecular biology 1135, 345-356 (2014). TNF (10 ng/ml) and oligomeric Aβ₁₋₄₂ (100 nM) were used for treating cells 18 hours. Cell viability was tested by luminescent ATP assay (FIG. 6A) and cell death was detected by DAPI and PI staining (FIG. 6B).

FIG. 7: N-acetyltransferase 1 (Nat1) expression decreases in the brain ECs of AD. Panel a shows that RNA-seq of brain ECs has revealed Nat1 is at the top of the down-regulated gene list during AD development. Fluorescence activated cell sorting (FACS) was used to isolate ECs (CD31^{high} and CD45^{low}) from single cell suspensions of brain tissues. The differentially expressed genes between APP/PS1 and WT groups were compared using edgeR and 116 genes were identified. In the plot, green points indicate genes that are down-regulated in AD mice while red points represent up-regulated genes. Panel b demonstrates that quantitative PCR (qPCR) has confirmed the decease of Nat1 mRNA expression in the isolated brain ECs of APP/PS1 mice. Panels c and d show the expression of Nat1 protein (mouse)/Nat2 protein (human) in brain microvessels decreases during AD pathogenesis. Microvessels from mouse or human brains were isolated as described in Munikoti, et al. Journal of neuroscience methods 207, 80-85 (2012) and Boulay et al. Journal of visualized experiments: JoVE, e53208 (2015). Panel e demonstrates that exogenous insulin increases Nat1 mRNA expression in cultured mouse brain ECs (bEnd.3). Different concentrations of insulin were applied into the culture medium for 18 h. Panel f shows that Akt inhibitor and oligomeric Aβ₁₋₄₂ prevents insulin-induced Nat1 mRNA up-regulation. MK-2206(5 uM, Akt inhibitor), Repamycin (250 nM, mTOR inhibitor) or oligomeric Aβ₁₋₄₂ (100 nM) was applied to the culture medium of bEnd.3 cells half an hour prior to the treatment of insulin.

FIG. 8: Knockdown of Nat1 (Nat1-KD) in bEnd.3 cells promotes cell death with increasing Ripk1 activation and decreasing A20 protein expression. Panel a shows that Nat1-KD cells are susceptible to TNF-induced Ripk1 dependent cell death. Nat1-KD in bEnd.3 cells were conducted by lentivirus transfection with Nat1 shRNA. Ctrl group was transfected with lentivirus expressing scrambled shRNA. The efficiency of Nat-1 KD was confirmed by western blotting (WB). TNFα(100 ng/ml) was applied in Ctrl or Nat-KD cells for 24 h with or without Ripk1 inhibitor, Nec1s (10 uM). Cell death was quantified with SYTOX green staining. Panel b shows a global quantitative analysis of proteome in Nat1-KD cells. Using Tandem Mass Tag (TMT) isobaric labeling mass spectrometry (MS), we identified 7707 proteins in Nat1-KD and Ctrl bEND.3 cells. Among them, 265 proteins, labeled as red dots in the plot, are up-regulated in Nat1-KD cells, including amyloid precursor protein (APP) and β-secretase 2 (Bace2). 181 proteins labeled with green are down-regulate in Nat1-KD cells, including low density lipoprotein receptor-related protein 1 (LRP1) and A20. Panel c shows a gene set analysis of differently expressed proteins by GeneAnalytics. The proteins that are down-regulated in Nat1-KD cells are enriched at various metabolism pathways and AD dysregulated genes. The up-regulated proteins in Nat1-KD cells are enriched at the activation of NF-κB signaling, the disruption of BBB and apoptosis induced DNA fragment pathways and also at AD dysregulated genes. Panel d demonstrates that Nat1-KD increases Ripk1 activation under TNFα treatment and decreases A20 protein expression. Nat1-KD or Ctrl bEnd.3 cells were treated with Flag-TNFα (50 ng/ml) as the indicated time course. Ripk1 activation was examined by the blotting of p-S166 Ripk1 for cell lysates. The decreased A20 expression in Nat1-KD cell lysates, as indicated by the MS data, was confirmed with WB. Immunoprecipitation (IP) of flag-TNFα has revealed the up-regulation of Ripk1 ubiquitination in complex I of Nat1-KD cells. Also, the recruitment of A20 into complex I is decreased. Panel e shows that K63 ubiquitination of Ripk1 in complex I increases in Nat1-KD cells.

FIG. 9A-H: Decreased acetylation of A20 promotes the susceptibility of Nat-1 KD cells to TNFα induced cell death. FIG. 9A shows that damaged mitochondria are observed in Nat1-KD bEnd.3 cells. Mitochondria was stained with MitoTracker and imaged in live cells. The scale bar represents 20 and 5 um, respectively. FIG. 9B demonstrates that the concentration of acetyl coenzyme A (acetyl-CoA) is reduced in Nat1-KD cells. FIG. 9C shows the schematics for the experiment of comparing N-terminal acetylation and lysine acetylation of A20 between ctrl and Nat1-KD cells. For N-terminal acetylation, subtiligase reaction as described in Yi et al. Cell 146, 607-620 (2011) was used. Biotin was added into the free N-terminal residue. Enrichment in the amount of biotin pulldown suggests a decrease in N-terminal acetylation. For lysine acetylation, we first of all adjusted the protein concentrations to equalize A20 input in cell lysates of ctrl and Nat1-KD groups. Anti-acetylated lysine antibody was used for IP and anti-A20 antibody was used for WB. FIG. 9D demonstrates that N-terminal acetylation and lysine acetylation of A20 are both reduced in Nat1-KD cells. FIGS. 9E and 9F show that the decreased concentrations of acetyl-CoA and A20 in Nat1-KD cells are restored by the treatment of sodium acetate over 24 h. FIG. 9G demonstrates that sodium acetate supplementation in culture medium (100 mM) inhibits the over-activation of Ripk1 in Nat1-KD cells. Nat1-KD bEnd.3 cells were treated with flag-TNFα (100 ng/ml) as the indicated time course. IP of flag tag has shown Ripk1 ubiquitination in complex I decreases in Nat1-KD cells with sodium acetate treatment, along with reduced Ripk1 activation indicated by the phosphorylation of S166. FIG. 9H shows that sodium acetate treatment rescues TNFα-induced cell death in Nat1-KD cells.

FIG. 10: Nat1-KD in mouse embryonic fibroblasts (MEFs) promotes Ripk1 dependent apoptosis and necroptosis. Panel a shows MEFs transfected with lentivirus expressing Nat1-KD shRNA (KD1 or KD 2) or control shRNA (Ctrl). The knockdown efficiency of Nat1 was examined by WB. Panels b-d show MEFs treated with 200 nM (5Z)-7-oxozeaeno (5z7), 250 nM SM-164/20 uM Z-VAD(SZ) or 250 nM SM-164(S) alone for 0.5 h followed by TNFα (10 ng/ml) with or without Nec-1s (10 uM).

FIG. 11: Lysosomal degradation of A20 increases in Nat1-KD bEnd.3 cells. Panel a shows a qPCR of mRNA expression of A20 in Ctrl and Nat1-KD cells. Panels b and c show bEnd.3 cells treated with cycloheximide (CHX, 250 ug/ml) to investigate the degradation kinetics of A20. The half-life of A20 in Ctrl group is 19.4 h and in Nat1-KD is 10.0 h. Panel d demonstrates that MG132(10 uM), the proteasome inhibitor, reduces the protein expression of A20. Panels e and f show Chloroquine (CQ, 10 uM) and E64d (5 ug/ml) applied for inhibiting lysosomal protein degradation.

FIG. 12A-C: AAV mediated Nat1 expression in cerebral endothelial cells increases A20 expression and inhibits necroptosis in cerebral microvessels of APP/PS1 mice. FIG. 12A shows data from APP/PS1 mice at the age of 4 months old injected with AAVs that specially target cerebral endothelial cells and express Nat1. After 6 weeks, mice were sacrificed and cerebral microvessels were isolated. Nat1 and A20 protein expression levels were checked by western blot. FIGS. 12B and 12C show the markers of necroptosis in cerebral endothelial cells (identified by CD31 staining), p-RIPK3 and p-MLKL, stained and imaged. Quantifications of percentage of endothelial cells that are positive for p-RIPK3 or p-MLKL reveal expression of Nat1 in cerebral endothelial cells inhibits necroptosis

FIG. 13A-C: AAV mediated Nat1 expression in cerebral endothelial cells restores the loss of cerebral microvessels in APP/PS1 mice (FIG. 13A), prevents the infiltration of neutrophils into brain parenchyma (FIG. 13B) and the damage of blood brain barrier (FIG. 13C).

FIG. 14: AAV mediated Nat1 expression in cerebral endothelial cells increases Aβ clearance via Lrp1. Panel a shows that Lrp1 expression is decreased in Nat1 knock-down endothelial cells. Panel b demonstrates that the expression of Nat1 in cerebral endothelial cells increases Lrp1 expression in microvessels of APP/PS1 mice. Panels c and d show that Aβ expression was decreased in APP/PS1 mice that were injected with Nat1 AAV. Panel e demonstrates that Inflammatory cytokines, IFN and IL-1(3, were reduced in brains of APP/PS1 mice with Nat1 AAV injection.

FIG. 15: Cognitive deficits in APP/PS1 mice were rescued by Nat1 AAV injection as observed in Open Field test (Panel a), novel Y maze test (Panel b), water T maze test (Panel c) and Barnes Maze test (Panel d).

### CERTAIN DEFINITIONS

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be parenteral. In some embodiments, administration may be oral. In some embodiments, administration may be intracranial. In some embodiments, administration may be intracerebral. In some embodiments, administration may be intranasal. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: As used herein, the term “agent”, may be used to refer to a compound or entity of any chemical class including, for example, a polypeptide, nucleic acid, saccharide, lipid, small molecule, metal, or combination or complex thereof. As will be clear from context to those skilled in the art, in some embodiments, the term may be utilized to refer to an entity that is or comprises a cell or organism, or a fraction, extract, or component thereof. Alternatively or additionally, as those skilled in the art will understand in light of context, in some embodiments, the term may be used to refer to a natural product in that it is found in and/or is obtained from nature. In some embodiments, again as will be understood by those skilled in the art in light of context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. Some particular embodiments of agents that may be utilized herein include small molecules, antibodies, antibody fragments, aptamers, nucleic acids (e.g., siRNAs, shRNAs, DNA/RNA hybrids, antisense oligonucleotides, ribozymes), peptides, peptide mimetics, etc.

Agonist: As used herein, the term “agonist” refers to an agent that (i) increases or induces the effects of another agent; and/or (ii) increases or induces one or more biological events. Agonists may be or include agents of any chemical class including, for example, small molecules, polypeptides, nucleic acids, carbohydrates, lipids, metals, and/or any other entity that shows the relevant agonistic activity. An agonist may be direct (in which case it exerts its influence directly upon its target) or indirect (in which case it exerts its influence by other than binding to its target; e.g., by interacting with a regulator of the target, for example so that level or activity of the target is altered).

Antibody: As used herein, the term “antibody” includes but is not limited to polyclonal, monoclonal, humanized, chimeric, Fab fragments, Fv fragments, F(ab′) fragments and F(ab′)2 fragments, as well as single chain antibodies (scFv), fusion proteins and other synthetic proteins which comprise the antigen-binding site of the antibody. Antibodies can be made by the skilled person using methods and commercially available services and kits known in the art. Methods of preparation of monoclonal antibodies are well known in the art and include hybridoma technology and phage display technology. Further antibodies suitable for use in the present disclosure are described, for example, in the following publications: Antibodies A Laboratory Manual, Second edition. Edward A. Greenfield. Cold Spring Harbor Laboratory Press (Sep. 30, 2013); Making and Using Antibodies: A Practical Handbook, Second Edition. Eds. Gary C. Howard and Matthew R. Kaser. CRC Press (Jul. 29, 2013); Antibody Engineering: Methods and Protocols, Second Edition (Methods in Molecular Biology). Patrick Chames. Humana Press (Aug. 21, 2012); Monoclonal Antibodies: Methods and Protocols (Methods in Molecular Biology). Eds. Vincent Ossipow and Nicolas Fischer. Humana Press (Feb. 12, 2014); and Human Monoclonal Antibodies: Methods and Protocols (Methods in Molecular Biology). Michael Steinitz. Humana Press (Sep. 30, 2013)).

Antibodies may be produced by standard techniques, for example by immunization with the appropriate polypeptide or portion(s) thereof, or by using a phage display library. If polyclonal antibodies are desired, a selected mammal (e.g., mouse, rabbit, goat, horse, etc.) is immunized with an immunogenic polypeptide bearing a desired epitope(s), optionally haptenized to another polypeptide. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Serum from the immunized animal is collected and treated according to known procedures. If serum containing polyclonal antibodies to the desired epitope contains antibodies to other antigens, the polyclonal antibodies can be purified by immunoaffinity chromatography or any other method known in the art. Techniques for producing and processing polyclonal antisera are well known in the art.

Antisense oligonucleotide: As used herein, the term “antisense oligonucleotide” (ASO) means a nucleic acid agent, at least a portion of which is complementary to a target nucleic acid (e.g., mRNA) to be inhibited. Antisense oligonucleotides are generally single-stranded nucleic acids (either a DNA, RNA, or hybrid RNA-DNA molecule), which are complementary to a target nucleic acid sequence, such as a portion of a target mRNA. By binding to a target sequence, an RNA-RNA, a DNA-DNA, or RNA-DNA duplex is formed, thereby inhibiting the function or level of the target nucleic acid, such as by blocking the transcription, processing, poly(A) addition, replication, translation, or promoting inhibitory mechanisms of the cells, such as promoting mRNA degradation. In some embodiments, an antisense oligonucleotide is 10 to 40, 12 to 35, or 15 to 35 bases in length, or any integer in between. An antisense oligonucleotide can comprise one or more modified bases, such as 2-Aminopurine, 2,6-Diaminopurine (2-Amino-dA), 5-Promo dU, 5-Methyl dC, deoxylnosine, Locked Nucleic Acid (LNA), 5-Nitroindole, 2′-O-Methyl bases, Hydroxmethyl dC 2′ Fluoro bases. An antisense oligonucleotide can comprise one or more modified bonds, such as a phosphorothioate bond.

Approximately or about: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Associated with: Two events or entities are “associated” with one another, as that term is used herein, if the presence, level and/or form of one is correlated with that of the other. For example, a particular biological phenomenon (e.g., necroptosis in brain endothelial cells) is considered to be associated with a particular disease, disorder, or condition (e.g., a neurovascular or neurodegenerative disease or disorder such as Alzheimer's disease), if its presence correlates with incidence of and/or susceptibility of the disease, disorder, or condition (e.g., across a relevant population).

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Excipient: As used herein, “excipient” refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Fragment or portion: A “fragment” is a continuous portion of a polypeptide that is shorter than the original polypeptide. In certain embodiments, a fragment refers to a polypeptide having significant sequence identity to the original polypeptide over a continuous portion of the fragment that comprises at least 50%, preferably at least 60%, at least 70%, at least 80%, at least 90%, at least 95%, or more, of the length of the fragment or the length of the polypeptide, (whichever is shorter). In certain embodiments, a fragment refers to a polypeptide having substantial sequence identity to the original polypeptide over a continuous portion of the fragment that comprises at least 50%, preferably at least 60%, at least 70%, at least 80%, at least 90%, at least 95%, or more, of the length of the fragment or the length of the polypeptide, (whichever is shorter). In a non-limiting embodiment a fragment has at least 80% identity to the original sequence over a continuous portion of the fragment that comprises between 90% and 100% of the fragment, e.g., over 100% of the length of the fragment or the length of the polypeptide, (whichever is shorter). In another non-limiting embodiment a fragment has at least 80% identity to the original sequence over a continuous portion of the fragment that comprises between 90% and 100% of the fragment, e.g., over 100% of the length of the fragment or the length of the polypeptide, (whichever is shorter).

In certain embodiments, a fragment possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment may be determined by crystallizing the protein, which can be done using standard methods. Alternately, an NMR solution structure can be generated, also using standard methods. A modeling program such as MODELER (Sali, A. and Blundell, TL, J. Mol. Biol., 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32(Web Server issue):W522-5, Jul. 1, 2004). In many embodiments, one, more than one, or all biological functions or activities of a fragment is substantially similar to that of the corresponding biological function or activity of the original molecule. In certain embodiments the activity of a fragment may be at least 20%, at least 50%, at least 60%, at least 70%, at least 80%, or at least 90% of the activity of the original molecule, up to approximately 100%, approximately 125%, or approximately 150% of the activity of the original molecule. In certain embodiments, an activity of a fragment is such that the amount or concentration of the fragment needed to produce an effect is within 0.5 to 5-fold of the amount or concentration of the original molecule needed to produce that effect.

Increased, decreased, or reduced: As used herein, “increased,” “decreased,” or “reduced,” or grammatically comparable comparative terms, indicate values that are relative to a comparable reference measurement. For example, in some embodiments, an assessed value achieved with an agent of interest may be “increased” relative to that obtained with a comparable reference agent. Alternatively or additionally, in some embodiments, an assessed value achieved in a subject or system of interest may be “increased” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Interfering RNA: As used herein, the term “interfering RNA” refers to an RNA agent that can inhibit gene expression through the biological process of RNA interference (RNAi). RNA interference (RNAi) is a process of sequence-specific post-transcriptional gene silencing in animals initiated by double-stranded (dsRNA) that is homologous in sequence to the silenced gene. In some embodiments, an interfering RNA comprises an RNA or RNA-like structure typically containing 15-50 base pairs (such as about 18-25 base pairs) and having a nucleobase sequence identical (complementary) or nearly identical (substantially complementary) to a coding sequence in an expressed target gene within a cell. Interfering RNAs include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207). siRNAs are generally RNA duplexes with each strand being 19-25 (such as 19-21) base pairs in length. Exemplary siRNAs are described by Hannon et al. Nature, 418 (6894): 244-51 (2002); Brummelkamp et al, Science 21, 21 (2002); and Sui et al, Proc. Natl Acad. Sci. USA 99, 5515-5520 (2002). An shRNA typically contains of a stem of 19-29 base pairs, a loop of at least 4 nucleotides (nt), and optionally a dinucleotide overhang at the 3′ end. Expression of shRNA in a subject can be obtained by delivery of a vector (e.g., a plasmid or viral or bacterial vectors) encoding the shRNA. siRNAs and shRNAs may be designed using any method known in the art, for example, based on the known target sequences. Interfering RNA agents may also comprise one or more chemical modifications, such as a base modification and/or a bond modification to at least improve its stability and binding affinity to a target mRNA.

Inhibition: As used herein, the term “inhibition” or “inhibiting” refers to any degree of inhibition and is not limited to only total inhibition. Thus, any degree of partial inhibition or relative reduction is intended to be included within the scope of the term “inhibition.” For example, inhibiting necroptosis includes complete inhibition of necroptosis or a reduction in necroptosis.

Inhibitor: As used herein, the term “inhibitor” refers to an agent whose presence or level correlates with decreased level or activity of a target to be modulated. In some embodiments, an inhibitor may be act directly (in which case it exerts its influence directly upon its target, for example by binding to the target); in some embodiments, an inhibitor may act indirectly (in which case it exerts its influence by interacting with and/or otherwise altering a regulator of a target, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.).

Nanoparticle: As used herein, a “nanoparticle” is a particle of submicron dimensions. Optionally, the nanoparticle is comprised of polymeric materials. Suitably, the nanoparticle may be comprised of natural or synthetic polymeric materials. As used herein, “synthetic polymeric materials” do not include natural polymers, such as proteins or starch. Examples of suitable polymeric materials include, but are not limited to homopolymers, copolymers, random polymers, graft polymers, alternating polymers, block polymers, branch polymers, arborescent polymers and dendritic polymers. Nanoparticles include nanospheres, which are nanoparticles having a substantially round, spherical or globular structure. Nanoparticles may be used to carry therapeutic agents for delivery to target cells or tissue. As used herein, carrying of a therapeutic agent by a nanoparticle includes encapsulation of the therapeutic agent by the nanoparticle, or attachment, adsorption or other association of the therapeutic agent to or with the nanoparticle. Suitably, nanoparticles may be biodegradable, for example being made of FDA-approved polymers and reagents for internal use. Nanoparticles may optionally comprise surface ligands that enhance their transfer to target cells such as endothelial cells of the brain. Suitably, nanoparticles may be at least 20 nm, at least 25 nm, at least 35 nm, at least 50 nm or at least 75 nm in average diameter. Suitably, nanoparticles may be less than 600 nm, less than 500 nm, less than 300 nm, less than 250 nm, less than 200 nm, less than 150 nm or less than 100 nm in average diameter. Suitably, nanoparticles are of a size that does not induce an inflammatory response in the target cell.

Neurodegenerative disease or disorder: The term “neurodegenerative disease or disorder” as used herein refers to a neurological disorder or disease, which is characterized by a gradual and progressive loss of neural tissue, and/or altered neurological function, typically reduced neurological function as a result of a gradual and progressive loss of neural tissue. In some embodiments, neurodegenerative diseases that are amenable to prevention and/or treatment using the methods described herein are neurodegenerative diseases that involve dysfunctional endothelial cells that form the blood-brain-barrier and/or dysfunctional cerebrovascular endothelial cells. Examples of such neurodegenerative diseases include, but are not limited to Alzheimer's Disease, Huntington's Disease, Parkinson's Disease, vascular dementia and the like. In some embodiments a neurodegenerative disease or disorder may be characterized by one or more symptoms associated with one or more disturbances in the normal functioning of cerebral vascular system in a subject, and is thus also considered as a neurovascular disease or disorder.

Neurovascular disease or disorder: As used interchangeably herein, the term “neurovascular disease or disorder” or “cerebrovascular disease or order” refers to a disorder characterized by one or more disturbances in the normal functioning of at least one component of the cerebral vascular or cerebral nervous system in a subject, e.g., a human. In some embodiments, the neurovascular disease or disorder is stroke. In some embodiments, the neurovascular disease or disorder is Alzheimer's disease.

Nucleic acid: As used herein, the term “nucleic acid” refers to a polymer of at least 10 nucleotides or more. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguano sine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of size, e.g., of an RNA oligonucleotide, a certain number of nucleotides refers to the number of nucleotides on a single strand, e.g., of an RNA oligonucleotide.

Polypeptide: The term “polypeptide”, as used herein, generally has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject.

Prevent or prevention: As used herein, “prevent” or “prevention,” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Reference: As used herein, “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. In some embodiments, a reference or control in the context of a reference level of a target refers to a level of a target in a normal healthy subject or a population of normal healthy subjects. In some embodiments, a reference or control in the context of a reference level of a target refers to a level of a target in a subject prior to a treatment. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

RNA agent: As used herein, the term “RNA agent” refers to an agent comprising ribonucleotides. In some embodiments, an RNA agent is single stranded. In some embodiments, an RNA agent is double stranded. In some embodiments, an RNA agent comprises both single and double stranded portions. In some embodiments, an RNA agent can comprise a backbone structure as described in the definition of “Nucleic acid” above. An RNA agent can be a regulatory RNA (e.g., siRNA, shRNA microRNA, etc.), or a messenger RNA (mRNA) oligonucleotide.

Selective or specific: The term “selective” or “specific”, when used herein with reference to an agent having an activity, is understood by those skilled in the art to mean that the agent discriminates between potential target entities, states, or cells. For example, in some embodiments, an agent is said to target “specifically” certain cells, e.g., brain endothelial cells or cerebral endothelial cells, if it acts preferentially on the certain cells in the presence of other cell types, e.g., non-endothelial cells. In some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments, specificity is evaluated relative to that of a reference non-specific binding agent. In some embodiments, the agent or entity does not detectably bind to the competing alternative target under conditions of binding to its target entity. In some embodiments, a binding agent binds with higher on-rate, lower off-rate, increased affinity, decreased dissociation, and/or increased stability to its target entity as compared with the competing alternative target(s).

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is biologically active. In some embodiments, suitable small molecules may be identified by methods such as screening large libraries of compounds (Beck-Sickinger & Weber (2001) Combinational Strategies in Biology and Chemistry (John Wiley & Sons, Chichester, Sussex); by structure-activity relationship by nuclear magnetic resonance (Shuker et al (1996) “Discovering high-affinity ligands for proteins: SAR by NMR.” Science 274: 1531-1534); encoded self-assembling chemical libraries (Melkko et al (2004) “Encoded self-assembling chemical libraries.” Nature Biotechnol. 22: 568-574); DNA-templated chemistry (Gartner et al (2004) “DNA-templated organic synthesis and selection of a library of macrocycles.” Science 305: 1601-1605); dynamic combinatorial chemistry (Ramstrom & Lehn (2002) “Drug discovery by dynamic combinatorial libraries.” Nature Rev. Drug Discov. 1: 26-36); tethering (Arkin & Wells (2004) “Small-molecule inhibitors of protein-protein interactions: progressing towards the dream.” Nature Rev. Drug Discov. 3: 301-317); and speed screen (Muckenschnabel et al (2004) “SpeedScreen: label-free liquid chromatography-mass spectrometry-based high-throughput screening for the discovery of orphan protein ligands.” Anal. Biochem. 324: 241-249). In some embodiments, a small molecule may have a dissociation constant for a target in the nanomolar range. In some embodiments, a small molecule is a small molecule that can undergo passive diffusion through the brain capillary endothelial wall, which makes up the blood-brain-barrier. In some embodiments, a small molecule is a small molecule that is actively transported via a lipid transporter present in brain endothelial cells. For example, such a small molecule may be a lipid-soluble and has a molecular weight less than 400-600 Da. Additional information about development of neuropharmaceuticals can be found, e.g., in Pardridge, Advanced Drug Delivery Reviews, 15: 5-35 (1995), which describes the biology of small molecule transport through the BBB, and also provides various methodologies for assessing whether small molecules undergo significant transport through the blood-brain-barrier in vivo. The contents of the aforementioned reference are incorporated by reference in their entireties for the purposes described herein.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Subject: As used herein, the term “subject” refers to an organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans, etc.). In some embodiments, a subject may be suffering from, and/or susceptible to a neurodegenerative or neurovascular disease, disorder, and/or condition.

Substantially: As used herein, the term “substantially” refers to the qualitative condition of exhibiting total or near-total extent or degree of a characteristic or property of interest. For example, “substantially complementary” in the context of degree of complementarity between two nucleotide sequences intends to encompass 100% complementarity and a few mismatches between two nucleotide sequences such that they are still able to be hybridized to each other.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the phrase “therapeutic agent” refers to an agent (e.g., a provided compound) that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature.

Vascular dementia: As used herein, the term “vascular dementia” is also referred to as “multi-infarct dementia” in the art refers to a group of syndromes caused by different mechanisms all resulting in vascular lesions in the brain. The main subtypes of vascular dementia are, for example vascular mild cognitive impairment, multi-infarct dementia, vascular dementia due to a strategic single infarct (affecting the thalamus, the anterior cerebral artery, the parietal lobes or the cingulate gyrus), vascular dementia due to hemorrhagic lesions, small vessel disease (including, e.g. vascular dementia due to lacunar lesions and Binswanger disease), and mixed Alzheimer's Disease with vascular dementia.

Vector: As used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

Alzheimer's disease (AD) is a multifactorial disease with majority of dementia patients displaying cerebrovascular pathology. However, the pathological mechanism that mediates the brain endothelial cells in AD is unclear.

The present disclosure is based at least in part, on an unexpected discovery that endothelial cell necroptosis in brain can contribute to development of a neurovascular or neurodegenerative disease or disorder, e.g., AD. Modulation of expression and/or activity of one or more molecular mediators involved in a necroptosis signaling pathway in the brain can, in turn, reduce neuroinflammation and/or the severity of cognitive decline in AD subjects. The present disclosure also encompasses the surprising discovery that N-acetyltransferase 1 (Nat1) or N-acetyltransferase 2 (Nat2) is an upstream regulator of a necroptosis signaling pathway involving tumor necrosis factor alpha-induced protein 3 (Tnfaip3/A20), receptor-interacting serine/threonine-protein kinase 1 (RIPK1), and receptor-interacting serine/threonine-protein kinase 3 (RIPK3). The present disclosure also encompasses the surprising discovery that inhibiting expression of receptor-interacting serine/threonine-protein kinase 3 (RIPK3), inhibiting expression of receptor-interacting serine/threonine-protein kinase 1 (RIPK1), and/or inducing expression of N-acetyltransferase 1 (Nat1) or N-acetyltransferase 2 (Nat2) in brain endothelial cells of an animal model of AD can reduce the severity of cognitive decline of the AD animals. Additionally, the present disclosure encompasses the unexpected discovery that increasing expression and/or activity of low density lipoprotein receptor-related protein 1 (LRP1) can increase clearance of AP, which can lead to decreased Nat1 or Nat2 levels in brain endothelial cells of an animal model of AD and reduce the severity of cognitive decline of the AD animals.

Accordingly, the present disclosure provides methods and compositions (including agents) for treating a neurovascular or neurodegenerative disease or disorder, which involve inhibiting or reducing necroptosis in brain endothelial cells of a subject. Necroptosis in brain endothelial cells of a subject can be inhibited, e.g., by (a) inhibiting or decreasing expression of RIPK3, (b) inhibiting or decreasing expression of RIPK1, (c) increasing expression of Nat1 or Nat2; (d) increasing expression of tumor necrosis factor alpha-induced protein 3 (Tnfaip3/A20), and/or (e) increasing expression of low-density lipoprotein receptor-related protein 1(LRP1). The present disclosure also provides methods and compositions (including agents) for inhibiting necroptosis in the brain of a subject in need thereof, which involve increasing expression of Nat1 or Nat2 in the brain of the subject.

### Agents for Inhibiting or Reducing Necroptosis

Receptor-interacting serine/threonine-protein kinase 1 (RIPK1), RIPK3, and mixed lineage kinase domain-like (MLKL) are known to be the key molecules in the necroptosis signaling pathway (Cho et al., Cell 137: 1112-1123 (2009); Sun et al. Cell 148: 213-227 (2012)). Many stimuli, such as tumor necrosis factor (TNF), Fas ligand (FasL), TNF-related apoptosis-inducing ligand (TRAIL), interferon (IFN), double-strand RNA (dsRNA) and lipopolysaccharide (LPS) can trigger necroptosis in cells (Jouan-Lanhouet et al. Cell Dev. Biol. 35: 2-13 (2014)).

In some embodiments, necroptosis is TNF-induced necroptosis. For example, the ligation of TNF receptor 1 (TNFR1) by TNF promotes the recruitment of TNFR1-associated death domain protein (TRADD), TNF receptor-associated factor 2 (TRAF2), RIPK1, and cellular inhibitor of apoptosis 1 and 2 (cIAP1/2) to form receptor complex I (Chen et al. Science 296: 1634-1635 (2002)). The internalization of complex I following cleavage of ubiquitin from RIPK1 by deubiquitinating enzymes, such as A20 or CYLD, leads to the formation of complex II, which is composed of RIPK1, caspase-8, and Fas-associated protein with death domain (FADD) (Vanlangenakker et al. Cell. Death Dis. 2 (2011); Vandenabeele et al. Nat. Rev. Mol. Cell Biol. 11: 700-714 (2010); Christofferson et al. Curr. Opin. Cell Biol. 22, 263-268 (2010); and Hitomi et al. Cell 135: 1311-1323 (2008). When caspase activity is inhibited, RIPK1 and RIPK3 are recruited and activated by phosphorylation; the activated RIPK3 then phosphorylates MLKL (Cho et al., Cell 137: 1112-1123 (2009); Sun et al. Cell 148: 213-227 (2012); Li et al. Cell 150: 339-350 (2012); Rodriguez et al. Cell Death Differ. 23: 76-88 (2016)). Phosphorylated MLKL is oligomerized and translocated to the plasma membrane, which is an essential event in the induction of necroptosis (Cal et al. Cell Biol. 16: 55-65 (2014); Hildebrand et al. Proc. Natl. Acad. Sci. USA 111:15072-15077(2014))

Receptor-Interacting Serine/Threonine-Protein Kinase 3 (RIPK3)

In some aspects, the present disclosure provides agents for inhibiting or reducing expression and/or activity of receptor-interacting serine/threonine-protein kinase 3 (RIPK3) (referred to as “RIPK3 inhibitors” herein). These agents are useful for inhibiting or reducing necroptosis. Inhibition or reduction of necroptosis in brain endothelial cells can provide therapeutic benefits for a neurovascular and/or neurodegenerative disease or disorder (e.g., Alzheimer's disease).

RIPK3 is a member of the receptor-interacting protein (RIP) family of serine/threonine protein kinases, and contains a C-terminal domain unique from other RIP family members. An RIPK3 polypeptide is typically localized to the cytoplasm, and can undergo nucleocytoplasmic shuttling dependent on novel nuclear localization and export signals. It is a component of the tumor necrosis factor (TNF) receptor-I signaling complex, and can induce apoptosis and weakly activate the NF-kappaB transcription factor. RIPK3 is shown to interact with RIPK1. Yu et al. Curr Biol. 9 (10): 539-42 (1999) and Li et al. Cell 150 (2): 339-50 (2012).

Wild-type RIPK3 sequences of various species are available on the world wide web from the NCBI, including, e.g., but not limited human and mouse. For example, the nucleotide sequence encoding an isoform of human RIPK3 polypeptide is available at NCBI under GenBank Accession No. NM_006871 (SEQ ID NO: 1) and its corresponding amino acid sequence is provided under GenBank Accession N. NP_006862 (SEQ ID NO: 2).

The nucleotide sequence of SEQ ID NO: 1 is shown below:

| | |
|---|---|
| 1 | gcgggactgt agaggcgcct ataagggaag ttgttcagtc aactcggaaa aagggtaaca |
| 61 | acccggaaag tagactcacc gtcttggtct agagactgac ccctgcacag acagacccct |
| 121 | tcccctctct gcgaaaggac caagccccag aagtcactcc atctcctacg gctcgcaatt |
| 181 | tccagaggcc ccctggcacc ttccagcctg atgtcgtgcg tcaagttatg gcccagcggt |
| 241 | gcccccgccc ccttggtgtc catcgaggaa ctggagaacc aggagctcgt cggcaaaggc |
| 301 | gggttcggca cagtgttccg ggcgcaacat aggaagtggg gctacgatgt ggcggtcaag |
| 361 | atcgtaaact cgaaggcgat atccagggag gtcaaggcca tggcaagtct ggataacgaa |
| 421 | ttcgtgctgc gcctagaagg ggttatcgag aaggtgaact gggaccaaga tcccaagccg |
| 481 | gctctggtga ctaaattcat ggagaacggc tccttgtcgg ggctgctgca gtcccagtgc |
| 541 | cctcggccct ggccgctcct ttgccgcctg ctgaaagaag tggtgcttgg gatgttttac |
| 601 | ctgcacgacc agaacccggt gctcctgcac cgggacctca agccatccaa cgtcctgctg |
| 661 | gacccagagc tgcacgtcaa gctggcagat tttggcctgt ccacatttca gggaggctca |
| 721 | cagtcaggga cagggtccgg ggagccaggg ggcaccctgg gctacttggc cccagaactg |
| 781 | tttgttaacg taaaccggaa ggcctccaca gccagtgacg tctacagctt cgggatccta |
| 841 | atgtgggcag tgcttgctgg aagagaagtt gagttgccaa ccgaaccatc actcgtgtac |
| 901 | gaagcagtgt gcaacaggca gaaccggcct tcattggctg agctgcccca agccgggcct |
| 961 | gagactcccg gcttagaagg actgaaggag ctaatgcagc tctgctggag cagtgagccc |
| 1021 | aaggacagac cctccttcca ggaatgccta ccaaaaactg atgaagtctt ccagatggtg |
| 1081 | gagaacaata tgaatgctgc tgtctccacg gtaaaggatt tcctgtctca gctcaggagc |
| 1141 | agcaatagga gattttctat cccagagtca ggccaaggag ggacagaaat ggatggcttt |
| 1201 | aggagaacca tagaaaacca gcactctcgt aatgatgtca tggtttctga gtggctaaac |
| 1261 | aaactgaatc tagaggagcc tcccagctct gttcctaaaa aatgcccgag ccttaccaag |
| 1321 | aggagcaggg cacaagagga gcaggttcca caagcctgga cagcaggcac atcttcagat |
| 1381 | tcgatggccc aacctcccca gactccagag acctcaactt tcagaaacca gatgcccagc |
| 1441 | cctacctcaa ctggaacacc aagtcctgga ccccgaggga atcagggggc tgagagacaa |
| 1501 | ggcatgaact ggtcctgcag gaccccggag ccaaatccag taacagggcg accgctcgtt |
| 1561 | aacatataca actgctctgg ggtgcaagtt ggagacaaca actacttgac tatgcaacag |
| 1621 | acaactgcct tgcccacatg gggcttggca ccttcgggca aggggagggg cttgcagcac |
| 1681 | cccccaccag taggttcgca agaaggccct aaagatcctg aagcctggag caggccacag |
| 1741 | ggttggtata atcatagcgg gaaataaagc accttccaag cttgcctcca agagttacga |
| 1801 | gttaaggaag agtgccaccc cttgaggccc ctgacttcct tctagggcag tctggcctgc |
| 1861 | ccacaaactg actttgtgac ctgtccccca ggagtcaata aacatgatgg aatgctagtc |
| 1921 | aaaaaaaaaa aaaaaaaaaa |

The amino acid sequence of SEQ ID NO: 2 is shown below:

| | |
|---|---|
| 1 | mscvklwpsg apaplvsiee lenqelvgkg gfgtvfraqh rkwgydvavk ivnskaisre |
| 61 | vkamasldne fvlrlegvie kvnwdqdpkp alvtkfmeng slsgllqsqc prpwpllcrl |
| 121 | lkevvlgmfy lhdqnpvllh rdlkpsnvll dpelhvklad fglstfqggs qsgtgsgepg |
| 181 | gtlgylapel fvnvnrkast asdvysfgil mwavlagrev elptepslvy eavcnrqnrp |
| 241 | slaelpqagp etpgleglke lmqlcwssep kdrpsfqecl pktdevfqmv ennmnaayst |
| 301 | vkdflsqlrs snrrfsipes gqggtemdgf rrtienqhsr ndvmvsewln klnleeppss |
| 361 | vpkkcpsltk rsraqeeqvp qawtagtssd smaqppqtpe tstfrnqmps ptstgtpspg |
| 421 | prgnqgaerq gmnwscrtpe pnpvtgrplv niyncsgvqv gdnnyltmqq ttalptwgla |
| 481 | psgkgrglqh pppvgsqegp kdpeawsrpq gwynhsgk |

As another example, the nucleotide sequence encoding an isoform of mouse RIPK3 polypeptide is available at NCBI under GenBank Accession No. NM_001164108 and its corresponding amino acid sequence is provided under GenBank Accession No. NP_001157580.

An RIPK3 inhibitor is an agent that partially or fully blocks, inhibits, or neutralizes a biological activity of an RIPK3 polypeptide (e.g., by reducing expression, localization, and/or activity of RIPK3 polypeptide—e.g., an active form thereof). In some embodiments, RIPK3 inhibitors may include, e.g., one or more of RIPK3 antagonist antibodies (e.g., full length antibodies or antibody fragments, or agents that incorporate or include either), RIPK3 polypeptide fragments or variants (e.g., defective and/or dominant negative variant(s), whether produced, for example, by isolation, by chemical synthesis, by recombinant technologies, or otherwise) of native RIPK3 polypeptides, RIPK3 antisense oligonucleotides, RIPK3 inhibitory small molecules, etc. Methods for identifying RIPK3 inhibitors can comprise contacting a polypeptide with a candidate RIPK3 inhibitor and measuring a detectable change in one or more biological activities normally associated with an RIPK3 polypeptide.

In some embodiments, an RIPK3 inhibitor can be an agent (e.g., an antibody, an aptamer, or a small molecule) that interferes with the binding affinity and/or interaction activity of RIPK3. Alternatively or additionally, in some embodiments an RIPK3 inhibitor can be an agent (e.g., an inhibitory polynucleotide or oligonucleotide such as interfering RNA or antisense oligonucleotide) that suppresses transcription and/or translation of RIPK3, thereby reducing the mRNA/protein level of RIPK3. In some embodiments, an RIPK3 inhibitor (e.g., ones as described herein) may reduce RIPK3 level and/or transcript level in brain endothelial cells (e.g., cerebral endothelial cells), for example by an amount that may be at least 20% or more, e.g., 30%, 40%, 50%, 60%, 70%, 80%, 90%, 95% or above. In some embodiments, inhibitory activity of such an RIPK3 inhibitor can be determined by any of a variety of methods, e.g., measuring transcript level and/or or protein level using any methods known in the art, e.g., mRNA expression assays such as PCR, and/or protein assays such as ELISA or Western blot.

In some embodiments, an RIPK3 inhibitor may comprise an antibody agent that specifically binds to RIPK3; in some such embodiments, binding interferes with (e.g., neutralizes) RIPK3's ability to interact with one or more other proteins involved in necroptosis pathway. In some embodiments, an RIPK3 inhibitor is considered to specifically bind to RIPK3 if the inhibitor binds to RIPK3 with a greater affinity than for an irrelevant or non-target polypeptide. In some embodiments, an RIPK3 inhibitor may bind to RIPK3 with at least 5, or at least 10 or at least 50 times greater affinity than for the irrelevant non-target polypeptide. In some embodiments, an RIPK3 inhibitor may bind to RIPK3 with at least 100, or at least 1,000, or at least 10,000 times greater affinity than for an irrelevant or non-target polypeptide. Such binding may be determined by methods well known in the art, such surface plasmon resonance or competitive binding assays. In some embodiments, an RIPK3 inhibitor may have an affinity (as measured by a dissociation constant, KD) for RIPK3 of at least 10⁻⁷ M, 10⁻⁸ M, 10⁻⁹ M, 10⁻¹⁰ M, or 10⁻¹¹ M.

In some embodiments, an RIPK3 inhibitor is a small molecule agent such as a small molecule, which typically has a molecular weight less than 5,000 kDa. In some embodiments, a suitable small molecule may be one that binds RIPK3 as identified by screening one or more libraries of compounds. In some embodiments, an RIPK3 small molecule inhibitor may have a dissociation constant for RIPK3 in the nanomolar range.

In some embodiments, an RIPK3 inhibitor comprises an interfering RNA such as a small interfering RNA (siRNA) and short hairpin RNA (shRNA). In some embodiments, an RIPK3 inhibitor comprises an RNA agent whose nucleotide sequence is sufficiently complementary to that of an RIPK3 transcript, or portion thereof, that the RIPK3 inhibitor hybridizes to the RIPK3 transcript. In some embodiments, an RIPK3 inhibitor comprises a small interfering RNA (siRNA) that binds to the mRNA of RIPK3 and blocks its translation or degrades the mRNA via RNA interference.

In some embodiments, an RIPK3 inhibitor is or comprises a short hairpin RNA (shRNA) that is complementary to an RIPK3 nucleic acid (e.g., an RIPK3 mRNA). In some embodiments, an RIPK3 shRNA may contain a stem of 18-30 or 18-25 base pairs, a loop of at least 4 nucleotides (nt), and optionally a dinucleotide overhang at the 3′ end. Expression of an RIPK3 shRNA in a subject can be obtained by delivery of a vector (e.g., a plasmid or viral or bacterial vectors) encoding the shRNA. siRNAs and shRNAs may be designed using any method known in the art, for example, based on the known sequence of RIPK3. Interfering RNA agents may also comprise one or more chemical modifications, such as a base modification and/or a bond modification to at least improve its stability and binding affinity to an RIPK3 mRNA. In some embodiments, an RIPK3 shRNA comprises the nucleotide sequence of CCTCAGATTCCACATACTTTA.

In some embodiments, an RIPK3 inhibitor may be or comprise an antisense oligonucleotide that is substantially complementary to an RIPK3 nucleic acid (e.g., an RIPK3 mRNA). In some embodiments, an RIPK3 antisense oligonucleotide is 10 to 40, 12 to 35, or 15 to 35 bases in length, or any integer in between. In some embodiments, an RIPK3 antisense oligonucleotide can comprise one or more modified bases, such as 2-Aminopurine, 2,6-Diaminopurine (2-Amino-dA), 5-Bromo dU, 5-Methyl dC, deoxylnosine, Locked Nucleic Acid (LNA), 5-Nitroindole, 2′-O-Methyl bases, Hydroxmethyl dC, 2′ Fluoro bases. In some embodiments, an RIPK3 antisense oligonucleotide can comprise one or more modifications to the backbone structure, such as using one or more phosphorothioate bonds, morpholinos, and/or peptide nucleic acids (PNA), which all confer nuclease resistance for improved potency and/or activity of antisense oligonucleotide. In some embodiments, chemical modifications and delivery methods described in Schoch et al., Neuron 94: 1056-1070 (2017) may be used in antisense oligonucleotides described herein.

In some embodiments, an RIPK3 inhibitor may be or comprise a ribozyme that is complementary to an RIPK3 nucleic acid (e.g., an RIPK3 mRNA) and cleaves the RIPK3 nucleic acid. Ribozymes are RNA or RNA-protein complexes that cleave nucleic acids in a site-specific fashion. In some embodiments, an RIPK3 ribozyme may have specific catalytic domains that possess endonuclease activity. An RIPK3 ribozyme may be a synthetic ribozyme, e.g., having a separate hybridizing region and catalytic region, wherein the hybridizing region is designed to recognize an RIPK3 sequence.

Nucleic acid agents for RIPK3 inhibition, e.g., siRNAs, shRNAs, ribozymes, and antisense oligonucleotides as described herein may be complementary to an RIPK3 nucleic acid (e.g., an RIPK3 mRNA), or a portion thereof. In some embodiments, a nucleic acid agent for RIPK3 inhibition, e.g., siRNAs, shRNAs, ribozymes, and antisense oligonucleotides may be 100% complementary to an RIPK3 nucleic acid (e.g., an RIPK3 mRNA). In some embodiments, a nucleic acid agent for RIPK3 inhibition, e.g., siRNAs, shRNAs, ribozymes, and antisense oligonucleotides may be at least 70% (including, e.g., at least 80%, at least 90%, at least 95%, at least 98%, or at least 99% or higher) complementary to an RIPK3 nucleic acid (e.g., an RIPK3 mRNA).

In some embodiments, an RIPK3 inhibitor may be or comprise a non-antibody peptide or protein. An RIPK3 peptide or protein may comprise an amino acid sequence that interferes with RIPK3 signaling pathway, e.g., interaction with RIPK1 polypeptide. Proteins and peptides may be designed using any method known in the art, e.g., by screening libraries of proteins or peptides for binding to RIPK3 polypeptide or inhibition of RIPK3 polypeptide binding to a ligand or polypeptide, such as RIPK1 polypeptide.

The capability of an RIPK3 candidate agent, such as a small molecule, protein, or peptide, to bind to or interact with an RIPK3 polypeptide or fragment thereof may be measured by any method of detecting/measuring a protein/protein interaction or other compound/protein interaction. Suitable methods include methods such as, for example, yeast two-hybrid interactions, co-purification, ELISA, co-immunoprecipitation and surface plasmon resonance methods. Thus, the candidate compound may be considered capable of binding to the polypeptide or fragment thereof if an interaction may be detected between the candidate compound and the polypeptide or fragment thereof by ELISA, co-immunoprecipitation or surface plasmon resonance methods or by a yeast two-hybrid interaction or co-purification method, all of which are known in the art. Screening assays which are capable of high throughput operation are also contemplated. Examples may include cell based assays and protein-protein binding assays.

Receptor-Interacting Serine/Threonine-Protein Kinase 1 (RIPK1)

In some aspects, the present disclosure provides agents for inhibiting or reducing expression and/or activity of receptor-interacting serine/threonine-protein kinase 1 (RIPK1) (referred to as “RIPK1 inhibitors” herein). These agents are useful for inhibiting or reducing necroptosis, which can, in turn, provide therapeutic benefits for a neurovascular and/or neurodegenerative disease or disorder (e.g., Alzheimer's disease).

RIPK1 is the first member of the receptor-interacting protein (RIP) family of serine/threonine protein kinases. RIPK1 plays a role in inflammation and cell death in response to tissue damage, pathogen recognition, and as part of developmental regulation. Festjens et al. Cell Death and Differentiation 14(3): 400-10 (2007). In terms of cell death, RIPK1 plays a role in apoptosis and necroptosis. Some of the cell survival pathways RIPK1 involves NF-kappaB, Akt, and JNK. Lin, Necrotic Cell Death. Springer New York. pp. 23-43 (2014). RIPK1 is known to express in brains of AD patients. Ofengeim et al. Proceedings of the National Academy of Sciences of the United States of America. 114: E8788-E879 (2014). RIPK1 regulates not only necroptosis, but cell inflammation as well, and as a result it is involved in the regulation of microglial functions, e.g., those associated with the development of neurodegenerative diseases such as AD. Id.

Wild-type RIPK1 sequences of various species are available on the world wide web from the NCBI, including, e.g., but not limited human and mouse. For example, the nucleotide sequence encoding an isoform of human RIPK1 polypeptide is available at NCBI under GenBank Accession No. NM_003804 (SEQ ID NO: 3) and its corresponding amino acid sequence is provided under GenBank Accession N. NP_003795 (SEQ ID NO: 4).

The nucleotide sequence of SEQ ID NO: 3 is shown below:

| | |
|---|---|
| 1 | gaacgcgcgc gcctccccgg ctagtcccgg caggcggagc cccagttaga ctgctcgtca |
| 61 | agtgtgggaa aagctccgtg gcgtcacaag ctactatata aaaggcggtg cccgccgggg |
| 121 | ccgagtggga gtccgcggcg agcgcagcag cagggcccgg tcctgcgcct cgggagtcgg |
| 181 | cgtccaggct cggagcgcga cacggagact aggtggcagg gtacagctct gccggggggg |
| 241 | gaaaaagtgg taccattttg ggcgttcttg agcttcagaa tgcaaccaga catgtccttg |
| 301 | aatgtcatta agatgaaatc cagtgacttc ctggagagtg cagaactgga cagcggaggc |
| 361 | tttgggaagg tgtctctgtg tttccacaga acccagggac tcatgatcat gaaaacagtg |
| 421 | tacaaggggc ccaactgcat tgagcacaac gaggccctct tggaggaggc gaagatgatg |
| 481 | aacagactga gacacagccg ggtggtgaag ctcctgggcg tcatcataga ggaagggaag |
| 541 | tactccctgg tgatggagta catggagaag ggcaacctga tgcacgtgct gaaagccgag |
| 601 | atgagtactc cgctttctgt aaaaggaagg ataattttgg aaatcattga aggaatgtgc |
| 661 | tacttacatg gaaaaggcgt gatacacaag gacctgaagc ctgaaaatat ccttgttgat |
| 721 | aatgacttcc acattaagat cgcagacctc ggccttgcct cctttaagat gtggagcaaa |
| 781 | ctgaataatg aagagcacaa tgagctgagg gaagtggacg gcaccgctaa gaagaatggc |
| 841 | ggcaccctct actacatggc gcccgagcac ctgaatgacg tcaacgcaaa gcccacagag |
| 901 | aagtcggatg tgtacagctt tgctgtagta ctctgggcga tatttgcaaa taaggagcca |
| 961 | tatgaaaatg ctatctgtga gcagcagttg ataatgtgca taaaatctgg gaacaggcca |
| 1021 | gatgtggatg acatcactga gtactgccca agagaaatta tcagtctcat gaagctctgc |
| 1081 | tgggaagcga atccggaagc tcggccgaca tttcctggca ttgaagaaaa atttaggcct |
| 1141 | ttttatttaa gtcaattaga agaaagtgta gaagaggacg tgaagagttt aaagaaagag |
| 1201 | tattcaaacg aaaatgcagt tgtgaagaga atgcagtctc ttcaacttga ttgtgtggca |
| 1261 | gtaccttcaa gccggtcaaa ttcagccaca gaacagcctg gttcactgca cagttcccag |
| 1321 | ggacttggga tgggtcctgt ggaggagtcc tggtttgctc cttccctgga gcacccacaa |
| 1381 | gaagagaatg agcccagcct gcagagtaaa ctccaagacg aagccaacta ccatctttat |
| 1441 | ggcagccgca tggacaggca gacgaaacag cagcccagac agaatgtggc ttacaacaga |
| 1501 | gaggaggaaa ggagacgcag ggtctcccat gacccttttg cacagcaaag accttacgag |
| 1561 | aattttcaga atacagaggg aaaaggcact gcttattcca gtgcagccag tcatggtaat |
| 1621 | gcagtgcacc agccctcagg gctcaccagc caacctcaag tactgtatca gaacaatgga |
| 1681 | ttatatagct cacatggctt tggaacaaga ccactggatc caggaacagc aggtcccaga |
| 1741 | gtttggtaca ggccaattcc aagtcatatg cctagtctgc ataatatccc agtgcctgag |
| 1801 | accaactatc taggaaatac acccaccatg ccattcagct ccttgccacc aacagatgaa |
| 1861 | tctataaaat ataccatata caatagtact ggcattcaga ttggagccta caattatatg |
| 1921 | gagattggtg ggacgagttc atcactacta gacagcacaa atacgaactt caaagaagag |
| 1981 | ccagctgcta agtaccaagc tatctttgat aataccacta gtctgacgga taaacacctg |
| 2041 | gacccaatca gggaaaatct gggaaagcac tggaaaaact gtgcccgtaa actgggcttc |
| 2101 | acacagtctc agattgatga aattgaccat gactatgagc gagatggact gaaagaaaag |
| 2161 | gtttaccaga tgctccaaaa gtgggtgatg agggaaggca taaagggagc cacggtgggg |
| 2221 | aagctggccc aggcgctcca ccagtgttcc aggatcgacc ttctgagcag cttgatttac |
| 2281 | gtcagccaga actaaccctg gatgggctac ggcagctgaa gtggacgcct cacttagtgg |
| 2341 | ataaccccag aaagttggct gcctcagagc attcagaatt ctgtcctcac tgataggggt |
| 2401 | tctgtgtctg cagaaatttt gtttcctgta cttcatagct ggagaatggg gaaagaaatc |
| 2461 | tgcagcaaag gggtctcact ctgttgccag gctggtctca aacttctgga ctcaagtgat |
| 2521 | cctcccgcct cggccttcca aagtgctggg atatcaggca ctgagccact gcgcccagcc |
| 2581 | aacaatccgc tctgaggaaa gcgtaagcag gaagacctct taatggcata gcaccaataa |
| 2641 | aaaaatgact cctagttgtg tttggaaagg gagagaagag atgtctgagg aaggtcatgt |
| 2701 | tctttcagct tatggcattt cctagagttt tgttgaagca agaagaaaaa ctcagagaat |
| 2761 | ataaaatcaa cttttaaaat tgtgtgctct cttcttcacg taggctcctg ttaaaaacaa |
| 2821 | agtgcagtca gattctaagc cctgttcaga gacttcgtgg atcacagctg cagctcaccg |
| 2881 | ccacatcaca ggatccgtta acgttaatac ccaatactct gtcagccact gtaggctcta |
| 2941 | agaaccacgt gcagtcttca gcccattaaa ttatcgatta ttttttaatg aattgaattt |
| 3001 | atattgagtc ttcaaattaa ctgaatggat ttaaaggggt accaaggagg ggggaaacat |
| 3061 | cagaatttcc caggcagttg ttgcaaggaa ttggtactaa ccgtgactac aacaaaaatt |
| 3121 | cttgattgac ttttaaagtt atttcctggc attctggtac cttcacccag cctgagtgcc |
| 3181 | ctggagaggg aacaggaaat gctgatctct acccctgggt gagaccagaa cctcagggct |
| 3241 | gatactgttg agtggcttcc tcggtttact ctgtgtactg tgaaagtatt ttcatatttt |
| 3301 | ttctgtgtgc cagagtgaaa aaggacagct tctgagtgtg gtaattgtgc ctctagcacc |
| 3361 | cagcctttca aagcccacct gaaacctggg ggtggatgaa agaactagaa tagaagactg |
| 3421 | aagctgggta ggccgctcag tgtccactgg cattttgcta aaccgacaag gaaggctgtg |
| 3481 | tgcttagctc tccccagagg gagggcgaga agggtgtggt gatggtcaat ctggctgtcg |
| 3541 | gaacagattc tggtgtcttg ggctgataac agtgttgttg attctgattg tgaatcccct |
| 3601 | caactctagc agacacatac acacccctga aatggggctg cagagcaggc tgtctcagcc |
| 3661 | ttgccactgt cggcatctcg gcctgggtaa ttctgttgtg gggactgtcc tgttccttgt |
| 3721 | aggatgttta gtagcatccc tgcccccacc tactagatgc caggggcact gttctcccca |
| 3781 | gccccccgcc ccagttgtga caatagtctc taaacattgt caaatggtcc aaggaaaggg |
| 3841 | gaaaattgcc ccggttgaga agagcactgc tgtaaagtaa tgagcctcgg ctctcctgtc |
| 3901 | tgcacctgtc cggttactac ttggccacca cgcagccttg gctcctacag cccaaaaggg |
| 3961 | agaatggagg gaggctccag gctttgctgg aggggcctgg gtgagttctg tttgctcctt |
| 4021 | gtaccaccat ccaaatggtg ttatcaaatc tcttagattc caaagaggtt gaataattaa |
| 4081 | tgttcaaagg caagagggca aggcattttt taacactttt taaaataaaa atttatacca |
| 4141 | caactttaaa aaaaaaaaaa aaaaa |

The amino acid sequence of SEQ ID NO: 4 is shown below:

| | |
|---|---|
| 1 | mqpdmslnvi kmkssdfles aeldsggfgk vslcfhrtqg lmimktvykg pnciehneal |
| 61 | leeakmmnrl rhsrvvkllg viieegkysl vmeymekgnl mhvlkaemst plsvkgriil |
| 121 | eiiegmcylh gkgvihkdlk penilvdndf hikiadlgla sfkmwsklnn eehnelrevd |
| 181 | gtakknggtl yymapehlnd vnakpteksd vysfavvlwa ifankepyen aiceqqlimc |
| 241 | iksgnrpdvd diteycprei islmklcwea npearptfpg ieekfrpfyl sqleesveed |
| 301 | vkslkkeysn enavvkrmqs lqldcvavps srsnsateqp gslhssqglg mgpveeswfa |
| 361 | pslehpqeen epslqsklqd eanyhlygsr mdrqtkqqpr qnvaynreee rrrrvshdpf |
| 421 | aqqrpyenfq ntegkgtays saashgnavh qpsgltsqpq vlyqnnglys shgfgtrpld |
| 481 | pgtagprvwy rpipshmpsl hnipvpetny lgntptmpfs slpptdesik ytiynstgiq |
| 541 | igaynymeig gtssslldst ntnfkeepaa kyqaifdntt sltdkhldpi renlgkhwkn |
| 601 | carklgftqs qideidhdye rdglkekvyq mlqkwvmreg ikgatvgkla qalhqcsrid |
| 661 | llssliyvsq n |

As another example, the nucleotide sequence encoding an isoform of mouse RIPK1 polypeptide is available at NCBI under GenBank Accession No. NM_001359997 and its corresponding amino acid sequence is provided under GenBank Accession No. NP_001346926.

An RIPK1 inhibitor is an agent that partially or fully blocks, inhibits, or neutralizes a biological activity of an RIPK1 polypeptide (e.g., by reducing expression, localization, and/or activity of RIPK1 polypeptide—e.g., an active form thereof). In some embodiments, RIPK1 inhibitors may include, e.g., one or more of RIPK1 antagonist antibodies (e.g., full length antibodies or antibody fragments, or agents that incorporated or include either), RIPK1 polypeptide fragments or variants (e.g., defective and/or dominant negative variant(s), whether produced, for example, by isolation, by chemical synthesis, by recombinant technologies, or otherwise) of native RIPK1 polypeptides, RIPK1 antisense oligonucleotides, RIPK1 inhibitory small molecules, etc. Methods for identifying RIPK1 inhibitors can comprise contacting a polypeptide with a candidate RIPK1 inhibitor and measuring a detectable change in one or more biological activities normally associated with an RIPK1 polypeptide.

In some embodiments, an RIPK1 inhibitor can be an agent (e.g., an antibody, an aptamer, or a small molecule) that interferes with the binding affinity and/or interaction activity of RIPK1. Alternatively or additionally, in some embodiments an RIPK1 inhibitor can be an agent (e.g., an inhibitory polynucleotide or oligonucleotide such as interfering RNA or antisense oligonucleotide) that suppresses transcription and/or translation of RIPK1, thereby reducing the mRNA/protein level of RIPK1. In some embodiments, an RIPK1 inhibitor (e.g., ones as described herein) may reduce RIPK1 level and/or transcript level in brain endothelial cells (e.g., cerebral endothelial cells), for example by an amount that may be at least 20% or more, e.g., 30%, 40%, 50%, 60%, 70%, 80%, 90%, 95% or above. In some embodiments, inhibitory activity of such an RIPK1 inhibitor can be determined by any of a variety of methods, e.g., measuring transcript level and/or or protein level using any methods known in the art, e.g., mRNA expression assays such as PCR, and/or protein assays such as ELISA or Western blot.

In some embodiments, an RIPK1 inhibitor may comprise an antibody agent that specifically binds to RIPK1; in some embodiments, binding interferes with (e.g., neutralizes) RIPK1's ability to interact with one or more other proteins involved in necroptosis pathway. In some embodiments, an RIPK1 inhibitor is considered to specifically bind to RIPK1 if the inhibitor binds to RIPK1 with a greater affinity than for an irrelevant or non-target polypeptide. In some embodiments, an RIPK1 inhibitor may bind to RIPK1 with at least 5, or at least 10 or at least 50 times greater affinity than for the irrelevant non-target polypeptide. In some embodiments, an RIPK1 inhibitor may bind to RIPK1 with at least 100, or at least 1,000, or at least 10,000 times greater affinity than for an irrelevant or non-target polypeptide. Such binding may be determined by methods well known in the art, such surface plasmon resonance or competitive binding assays. In some embodiments, an RIPK1 inhibitor may have an affinity (as measured by a dissociation constant, KD) for RIPK1 of at least 10⁻⁷ M, 10⁻⁸ M, 10⁻⁹ M, 10⁻¹⁰ M, or 10⁻¹¹ M.

In some embodiments, an RIPK1 inhibitor is a small molecule agent such as a small molecule, which typically has a molecular weight less than 5,000 kDa. In some embodiments, a suitable small molecule may be one that binds RIPK1 and may be identified by screening large libraries of compounds.

Non-limiting examples of RIPK1 small molecule inhibitors include, but are not limited to ones described in WO2018/148626, WO 2018/109097, WO2016/101887, WO2016/101885. In some embodiments, an RIPK1 small molecule inhibitor is or comprises a compound as described in U.S. Provisional Application No. 62/750,211 entitled “Compounds and Methods for Inhibiting Necroptosis” and filed Oct. 24, 2018, the contents of which are incorporated herein by reference in its entireties.

In some embodiments, an RIPK1 inhibitor comprises an interfering RNA such as a small interfering RNA (siRNA) short hairpin RNA (shRNA). In some embodiments, an RIPK1 inhibitor comprises an RNA agent whose nucleotide sequence is sufficiently complementary to that of an RIPK1 transcript, or portion thereof, that the RIPK1 inhibitor hybridizes to the RIPK1 transcript. In some embodiments, an RIPK1 inhibitor comprises a small interfering RNA (siRNA) that binds to the mRNA of RIPK1 and blocks its translation or degrades the mRNA via RNA interference.

In some embodiments, an RIPK1 inhibitor is or comprises a short hairpin RNA (shRNA) that is complementary to an RIPK1 nucleic acid (e.g., an RIPK1 mRNA). In some embodiments, an RIPK1 shRNA may contain a stem of 18-30 or 18-25 base pairs, a loop of at least 4 nucleotides (nt), and optionally a dinucleotide overhang at the 3′ end. Expression of an RIPK1 shRNA in a subject can be obtained by delivery of a vector (e.g., a plasmid or viral or bacterial vectors) encoding the shRNA. RIPK1 siRNAs and shRNAs may be designed using any method known in the art, for example, based on the known sequence of RIPK1. RIPK1 interfering RNA agents may also comprise one or more chemical modifications, such as a base modification and/or a bond modification to at least improve its stability and binding affinity to an RIPK1 mRNA.

In some embodiments, an RIPK1 inhibitor may be or comprise an antisense oligonucleotide that is complementary to an RIPK1 nucleic acid (e.g., an RIPK1 mRNA). In some embodiments, an RIPK1 antisense oligonucleotide is 10 to 40, 12 to 35, or 15 to 35 bases in length, or any integer in between. In some embodiments, an RIPK1 antisense oligonucleotide can comprise one or more modified bases, such as 2-Aminopurine, 2,6-Diaminopurine (2-Amino-dA), 5-Bromo dU, 5-Methyl dC, deoxylnosine, Locked Nucleic Acid (LNA), 5-Nitroindole, 2′-O-Methyl bases, Hydroxmethyl dC, 2′ Fluoro bases. In some embodiments, an RIPK1 antisense oligonucleotide can comprise one or more modifications to the backbone structure, such as using one or more phosphorothioate bonds, morpholinos, and/or peptide nucleic acids (PNA), which all confer nuclease resistance for improved potency and/or activity of antisense oligonucleotide. In some embodiments, chemical modifications and delivery methods described in Schoch et al., Neuron 94: 1056-1070 (2017) may be used in antisense oligonucleotides described herein.

In some embodiments, an RIPK1 inhibitor may be or comprise a ribozyme that is complementary to an RIPK1 nucleic acid (e.g., an RIPK1 mRNA) and cleaves the RIPK1 nucleic acid. In some embodiments, an RIPK1 ribozyme may have specific catalytic domains that possess endonuclease activity. An RIPK1 ribozyme may be a synthetic ribozyme, e.g., having a separate hybridizing region and catalytic region, wherein the hybridizing region is designed to recognize an RIPK1 sequence.

Nucleic acid agents for RIPK1 inhibition, e.g., siRNAs, shRNAs, ribozymes, and antisense oligonucleotides as described herein may be complementary to an RIPK1 nucleic acid (e.g., an RIPK1 mRNA), or a portion thereof. In some embodiments, a nucleic acid agent for RIPK1 inhibition, e.g., siRNAs, shRNAs, ribozymes, and antisense oligonucleotides may be 100% complementary to an RIPK1 nucleic acid (e.g., an RIPK1 mRNA). In some embodiments, a nucleic acid agent for RIPK1 inhibition, e.g., siRNAs, shRNAs, ribozymes, and antisense oligonucleotides may be at least 70% (including, e.g., at least 80%, at least 90%, at least 95%, at least 98%, or at least 99% or higher) complementary to an RIPK1 nucleic acid (e.g., an RIPK1 mRNA).

In some embodiments, an RIPK1 inhibitor may be or comprise a non-antibody peptide or protein. An RIPK1 peptide or protein may comprise an amino acid sequence that interferes with RIPK1 signaling pathway, e.g., its interaction with RIPK3. Proteins and peptides may be designed using any method known in the art, e.g., by screening libraries of proteins or peptides for binding to RIPK1 polypeptide or inhibition of RIPK1 polypeptide binding to a ligand or polypeptide, such as RIPK3. The capability of an RIPK1 candidate agent, such as a small molecule, protein, or peptide, to bind to or interact with an RIPK1 polypeptide or fragment thereof may be measured by any method of detecting/measuring a protein/protein interaction or other compound/protein interaction.

N-acetyltransferase 1 (Nat1) or N-acetyltransferase 2 (Nat2)

The present disclosure recognizes, among others, that a reduction in N-acetyltransferase 1 (Nat1) or N-acetyltransferase 2 (Nat2) can lead to degradation of tumor necrosis factor alpha-induced protein 3 (A20, encoded by Tnfaip3), which is a negative regulator of RIPK1 activation and, in turn, sensitizes brain endothelial cells to TNF-alpha-induced cell death.

In some aspects, the present disclosure provides agents for increasing expression and/or activity of Nat1 or Nat2 (referred to as “Nat1 or Nat2 agonists” herein). These agents are useful for inhibiting or reducing necroptosis, which can, in turn, provide therapeutic benefits for a neurovascular and/or neurodegenerative disease or disorder (e.g., Alzheimer's disease).

N-acetyltransferase 2 (Nat2), also known as arylamine N-acetyltransferase, is an enzyme which in humans is encoded by Nat2 gene. Nat2 polypeptide generally functions to activate and deactivate arylamine and hydrazine drugs and carcinogens. Polymorphisms in Nat2 gene are reported to be responsible for the N-acetylation polymorphism in which human populations segregate into rapid, intermediate, and slow acetylator phenotypes. Polymorphisms in Nat2 gene are also reported to be associated with higher incidences of cancer and drug toxicity. A mouse homolog of Nat2 (in humans) is Nat1.

Wild-type Nat2 sequences of various species are available on the world wide web from the NCBI, including, e.g., but not limited human and mouse. For example, the nucleotide sequence encoding an isoform of human Nat2 polypeptide is available at NCBI under GenBank Accession No. NM_000015 (SEQ ID NO: 5) and its corresponding amino acid sequence is provided under GenBank Accession N. NP_000006 (SEQ ID NO: 6).

The nucleotide sequence of SEQ ID NO: 5 is shown below:

| | |
|---|---|
| 1 | tgagatcact tcccttgcag actttggaag ggagagcact ttattacaga ccttggaagc |
| 61 | aagaggattg cattcagcct agttcctggt tgctggccaa agggatcatg gacattgaag |
| 121 | catattttga aagaattggc tataagaact ctaggaacaa attggacttg gaaacattaa |
| 181 | ctgacattct tgagcaccag atccgggctg ttccctttga gaaccttaac atgcattgtg |
| 241 | ggcaagccat ggagttgggc ttagaggcta tttttgatca cattgtaaga agaaaccggg |
| 301 | gtgggtggtg tctccaggtc aatcaacttc tgtactgggc tctgaccaca atcggttttc |
| 361 | agaccacaat gttaggaggg tatttttaca tccctccagt taacaaatac agcactggca |
| 421 | tggttcacct tctcctgcag gtgaccattg acggcaggaa ttacattgtc gatgctgggt |
| 481 | ctggaagctc ctcccagatg tggcagcctc tagaattaat ttctgggaag gatcagcctc |
| 541 | aggtgccttg cattttctgc ttgacagaag agagaggaat ctggtacctg gaccaaatca |
| 601 | ggagagagca gtatattaca aacaaagaat ttcttaattc tcatctcctg ccaaagaaga |
| 661 | aacaccaaaa aatatactta tttacgcttg aacctcgaac aattgaagat tttgagtcta |
| 721 | tgaatacata cctgcagacg tctccaacat cttcatttat aaccacatca ttttgttcct |
| 781 | tgcagacccc agaaggggtt tactgtttgg tgggcttcat cctcacctat agaaaattca |
| 841 | attataaaga caatacagat ctggtcgagt ttaaaactct cactgaggaa gaggttgaag |
| 901 | aagtgctgag aaatatattt aagatttcct tggggagaaa tctcgtgccc aaacctggtg |
| 961 | atggatccct tactatttag aataaggaac aaaataaacc cttgtgtatg tatcacccaa |
| 1021 | ctcactaatt atcaacttat gtgctatcag atatcctctc taccctcacg ttattttgaa |
| 1081 | gaaaatccta aacatcaaat actttcatcc ataaaaatgt cagcatttat taaaaaacaa |
| 1141 | taacttttta aagaaacata aggacacatt ttcaaattaa taaaaataaa ggcattttaa |
| 1201 | ggatggcctg tgattatctt gggaagcaga gtgattcatg ctagaaaaca tttaatattg |
| 1261 | atttattgtt gaattcatag taaattttta ctggtaaatg aataaagaat attgtgg |

The amino acid sequence of SEQ ID NO: 6 is shown below:

| | |
|---|---|
| 1 | mdieayferi gyknsrnkld letltdileh qiravpfenl nmhcgqamel gleaifdhiv |
| 61 | rrnrggwclq vnqllywalt tigfqttmlg gyfyippvnk ystgmvhlll qvtidgrnyi |
| 121 | vdagsgsssq mwqplelisg kdqpqvpcif clteergiwy ldqirreqyi tnkeflnshl |
| 181 | lpkkkhqkiy lftleprtie dfesmntylq tsptssfitt sfcslqtpeg vyclvgfilt |
| 241 | yrkfnykdnt dlvefktlte eeveevlrni fkislgrnlv pkpgdgslti |

As another example, the nucleotide sequence encoding an isoform of a mouse homolog of human Nat2 (i.e., corresponding to Nat1 in mouse species) is available at NCBI under GenBank Accession No. NM_008673 and its corresponding amino acid sequence is provided under GenBank Accession No. NP_032699.

A Nat1 or Nat2 agonist is an agent that directly or indirectly stabilizes Nat1 or Nat2 polypeptide, e.g., partially or fully blocking or reducing degradation of Nat1 or Nat2 polypeptide or mRNA (which in turn increasing the half-life of Nat1 or Nat2 in brain endothelial cells); and/or directly or indirectly increases a biological activity of a Nat1 or Nat2 polypeptide (e.g., by increasing expression, localization, and/or activity of Nat1 or Nat2 polypeptide—e.g., an active form thereof). In some embodiments, Nat1 or Nat2 agonists may include, e.g., one or more of Nat1 or Nat2 agonistic antibodies (e.g., full length antibodies or antibody fragments, or agents that incorporate or include either), Nat1 or Nat2 polypeptide fragments or variants (e.g., polypeptide fragments or variants that retain a biological activity of native Nat1 or Nat2 polypeptides, whether produced, for example, by isolation, by chemical synthesis, by recombinant technologies, or otherwise) of native Nat1 or Nat2 polypeptides, Nat1 or Nat2 stimulatory small molecules, etc. Methods for identifying Nat1 or Nat2 agonists can comprise contacting a polypeptide with a candidate Nat1 or Nat2 agonist and measuring a detectable change in its level and/or one or more biological activities normally associated with a Nat1 or Nat2 polypeptide.

In some embodiments, a Nat1 or Nat2 agonist can be a molecule (e.g., an activating polynucleotide or oligonucleotide) that induces transcription and/or translation of Nat1 or Nat2, thereby increasing the mRNA/protein level of Nat1 or Nat2 polypeptide. In some embodiments, a Nat1 or Nat2 agonist described herein may increase the transcription and/or translation of Nat1 or Nat2 in brain endothelial cells (e.g., cerebral endothelial cells), for example, by an amount that may be at least 20% or more, e.g., 30%, 40%, 50%, 60%, 70%, 80%, 90%, 95% or above, as compared to transcription and/or translation of Nat1 or Nat2 in brain endothelial cells in the absence of the Nat1 or Nat2 agonist. In some embodiments, a Nat1 or Nat2 agonist described herein may increase the transcription and/or translation of Nat1 or Nat2 in brain endothelial cells (e.g., cerebral endothelial cells), for example, by an amount that may be at least 1.1-fold or more, e.g., 2-fold, 3-fold, 4-fold, 5-fold, or more, as compared to transcription and/or translation of Nat1 or Nat2 in brain endothelial cells in the absence of the Nat1 or Nat2 agonist. In some embodiments, activity of such a Nat1 or Nat2 agonist can be determined by any of a variety of methods, e.g., measuring transcript level and/or or protein level using any methods known in the art, e.g., mRNA expression assays such as PCR, and/or protein assays such as ELISA or Western blot.

In some embodiments, a Nat1 or Nat2 agonist may be or comprise a Nat1 or Nat2 polypeptide fragment or variant of a native Nat1 or Nat2 polypeptide that retains at least 30% or more (including, e.g., at least 40%, at least 50%, at least 60%, at least 70%, at least 80%, at least 90%, or more) of a biological activity of a native Nat1 or Nat2 polypeptide, whether produced, for example, by isolation, by chemical synthesis, by recombinant technologies, or otherwise.

In some embodiments, a Nat1 or Nat2 agonist may be or comprise a Nat1 or Nat2 peptidomimetic (e.g., peptoid).

In some embodiments, a Nat1 or Nat2 agonist may comprise an expression vector comprising nucleic acid agent that encodes a Nat1 or Nat2 or a functional fragment thereof.

Tumor Necrosis Factor Alpha-Induced Protein 3 (Tnfaip3/A20)

In some aspects, the present disclosure provides agents for increasing expression and/or activity of tumor necrosis factor alpha-induced protein 3 (Tnfaip3/A20) (referred to as “A20 agonist” herein). These agents are useful for inhibiting or reducing necroptosis, which can, in turn, provide therapeutic benefits for a neurovascular and/or neurodegenerative disease or disorder (e.g., Alzheimer's disease).

Tumor necrosis factor alpha-induced protein 3 is an ubiquitin-editing enzyme that contains both ubiquitin ligase and deubiquitinase activities, and it is also known as a zinc finger protein A20. It is known to be involved in immune and inflammatory responses signaled by cytokines, such as TNF-alpha and IL-1 beta, or pathogens via Toll-like receptors (TLRs) through terminating NF-kappa-B activity. Upon TNF stimulation, deubiquitinates “Lys-63”-polyubiquitin chains on RIPK1 and catalyzes the formation of “Lys-48”-polyubiquitin chains. This leads to RIPK1 proteasomal degradation and consequently termination of the TNF- or LPS-mediated activation of NF-kappa-B.

Wild-type TNF alpha induced protein 3 sequences of various species are available on the world wide web from the NCBI, including, e.g., but not limited to human. For example, the nucleotide sequence encoding an isoform of human TNF alpha induced protein 3 polypeptide is available at NCBI under GenBank Accession No. NM_001270507 (SEQ ID NO: 7) and its corresponding amino acid sequence is provided under GenBank Accession N. NP_001257436 (SEQ ID NO: 8).

The nucleotide sequence of SEQ ID NO: 7 is shown below:

| | |
|---|---|
| 1 | ctttggaaag tcccgtggaa atccccgggc ctacaacccg catacaactg aaacggggca |
| 61 | aagcagactg cgcagtctgc agtcttcgtg gcgggccaag cgagcttgga gcccgcgggg |
| 121 | gcggagcggt gagagcggcc gccaagagag atcacacccc cagccgaccc tgccagcgag |
| 181 | cgagcccgac cccaggcgtc catggagcgt cgcctccgcc cggtccctgc cccgaccccc |
| 241 | gcctgcggcg cgctcctgcc ttgaccagga cttgggactt tgcgaaagga tcgcggggcc |
| 301 | cggagaggta accgccgcgc ctcccggaga ggtgttggag agcacaatgg ctgaacaagt |
| 361 | ccttcctcag gctttgtatt tgagcaatat gcggaaagct gtgaagatac gggagagaac |
| 421 | tccagaagac atttttaaac ctactaatgg gatcattcat cattttaaaa ccatgcaccg |
| 481 | atacacactg gaaatgttca gaacttgcca gttttgtcct cagtttcggg agatcatcca |
| 541 | caaagccctc atcgacagaa acatccaggc caccctggaa agccagaaga aactcaactg |
| 601 | gtgtcgagaa gtccggaagc ttgtggcgct gaaaacgaac ggtgacggca attgcctcat |
| 661 | gcatgccact tctcagtaca tgtggggcgt tcaggacaca gacttggtac tgaggaaggc |
| 721 | gctgttcagc acgctcaagg aaacagacac acgcaacttt aaattccgct ggcaactgga |
| 781 | gtctctcaaa tctcaggaat ttgttgaaac ggggctttgc tatgatactc ggaactggaa |
| 841 | tgatgaatgg gacaatctta tcaaaatggc ttccacagac acacccatgg cccgaagtgg |
| 901 | acttcagtac aactcactgg aagaaataca catatttgtc ctttgcaaca tcctcagaag |
| 961 | gccaatcatt gtcatttcag acaaaatgct aagaagtttg gaatcaggtt ccaatttcgc |
| 1021 | ccctttgaaa gtgggtggaa tttacttgcc tctccactgg cctgcccagg aatgctacag |
| 1081 | ataccccatt gttctcggct atgacagcca tcattttgta cccttggtga ccctgaagga |
| 1141 | cagtgggcct gaaatccgag ctgttccact tgttaacaga gaccggggaa gatttgaaga |
| 1201 | cttaaaagtt cactttttga cagatcctga aaatgagatg aaggagaagc tcttaaaaga |
| 1261 | gtacttaatg gtgatagaaa tccccgtcca aggctgggac catggcacaa ctcatctcat |
| 1321 | caatgccgca aagttggatg aagctaactt accaaaagaa atcaatctgg tagatgatta |
| 1381 | ctttgaactt gttcagcatg agtacaagaa atggcaggaa aacagcgagc aggggaggag |
| 1441 | agaggggcac gcccagaatc ccatggaacc ttccgtgccc cagctttctc tcatggatgt |
| 1501 | aaaatgtgaa acgcccaact gccccttctt catgtctgtg aacacccagc ctttatgcca |
| 1561 | tgagtgctca gagaggcggc aaaagaatca aaacaaactc ccaaagctga actccaagcc |
| 1621 | gggccctgag gggctccctg gcatggcgct cggggcctct cggggagaag cctatgagcc |
| 1681 | cttggcgtgg aaccctgagg agtccactgg ggggcctcat tcggccccac cgacagcacc |
| 1741 | cagccctttt ctgttcagtg agaccactgc catgaagtgc aggagccccg gctgcccctt |
| 1801 | cacactgaat gtgcagcaca acggattttg tgaacgttgc cacaacgccc ggcaacttca |
| 1861 | cgccagccac gccccagacc acacaaggca cttggatccc gggaagtgcc aagcctgcct |
| 1921 | ccaggatgtt accaggacat ttaatgggat ctgcagtact tgcttcaaaa ggactacagc |
| 1981 | agaggcctcc tccagcctca gcaccagcct ccctccttcc tgtcaccagc gttccaagtc |
| 2041 | agatccctcg cggctcgtcc ggagcccctc cccgcattct tgccacagag ctggaaacga |
| 2101 | cgcccctgct ggctgcctgt ctcaagctgc acggactcct ggggacagga cggggacgag |
| 2161 | caagtgcaga aaagccggct gcgtgtattt tgggactcca gaaaacaagg gcttttgcac |
| 2221 | actgtgtttc atcgagtaca gagaaaacaa acattttgct gctgcctcag ggaaagtcag |
| 2281 | tcccacagcg tccaggttcc agaacaccat tccgtgcctg gggagggaat gcggcaccct |
| 2341 | tggaagcacc atgtttgaag gatactgcca gaagtgtttc attgaagctc agaatcagag |
| 2401 | atttcatgag gccaaaagga cagaagagca actgagatcg agccagcgca gagatgtgcc |
| 2461 | tcgaaccaca caaagcacct caaggcccaa gtgcgcccgg gcctcctgca agaacatcct |
| 2521 | ggcctgccgc agcgaggagc tctgcatgga gtgtcagcat cccaaccaga ggatgggccc |
| 2581 | tggggcccac cggggtgagc ctgcccccga agaccccccc aagcagcgtt gccgggcccc |
| 2641 | cgcctgtgat cattttggca atgccaagtg caacggctac tgcaacgaat gctttcagtt |
| 2701 | caagcagatg tatggctaac cggaaacagg tgggtcacct cctgcaagaa gtggggcctc |
| 2761 | gagctgtcag tcatcatggt gctatcctct gaacccctca gctgccactg caacagtggg |
| 2821 | cttaagggtg tctgagcagg agaggaaaga taagctcttc gtggtgccca cgatgctcag |
| 2881 | gtttggtaac ccgggagtgt tcccaggtgg ccttagaaag caaagcttgt aactggcaag |
| 2941 | ggatgatgtc agattcagcc caaggttcct cctctcctac caagcaggag gccaggaact |
| 3001 | tctttggact tggaaggtgt gcggggactg gccgaggccc ctgcaccctg cgcatcagga |
| 3061 | ctgcttcatc gtcttggctg agaaagggaa aagacacaca agtcgcgtgg gttggagaag |
| 3121 | ccagagccat tccacctccc ctcccccagc atctctcaga gatgtgaagc cagatcctca |
| 3181 | tggcagcgag gccctctgca agaagctcaa ggaagctcag ggaaaatgga cgtattcaga |
| 3241 | gagtgtttgt agttcatggt ttttccctac ctgcccggtt cctttcctga ggacccggca |
| 3301 | gaaatgcaga accatccatg gactgtgatt ctgaggctgc tgagactgaa catgttcaca |
| 3361 | ttgacagaaa aacaagctgc tctttataat atgcaccttt taaaaaatta gaatatttta |
| 3421 | ctgggaagac gtgtaactct ttgggttatt actgtcttta cttctaaaga agttagcttg |
| 3481 | aactgaggag taaaagtgtg tacatatata atataccctt acattatgta tgagggattt |
| 3541 | ttttaaatta tattgaaatg ctgccctaga agtacaatag gaaggctaaa taataataac |
| 3601 | ctgttttctg gttgttgttg gggcatgagc ttgtgtatac actgcttgca taaactcaac |
| 3661 | cagctgcctt tttaaaggga gctctagtcc tttttgtgta attcacttta tttattttat |
| 3721 | tacaaacttc aagattattt aagtgaagat atttcttcag ctctggggaa aatgccacag |
| 3781 | tgttctcctg agagaacatc cttgctttga gtcaggctgt gggcaagttc ctgaccacag |
| 3841 | ggagtaaatt ggcctctttg atacactttt gcttgcctcc ccaggaaaga aggaattgca |
| 3901 | tccaaggtat acatacatat tcatcgatgt ttcgtgcttc tccttatgaa actccagcta |
| 3961 | tgtaataaaa aactatactc tgtgttctgt taatgcctct gagtgtccta cctccttgga |
| 4021 | gatgagatag ggaaggagca gggatgagac tggcaatggt cacagggaaa gatgtggcct |
| 4081 | tttgtgatgg ttttattttc tgttaacact gtgtcctggg ggggctggga agtcccctgc |
| 4141 | atcccatggt accctggtat tgggacagca aaagccagta accatgagta tgaggaaatc |
| 4201 | tctttctgtt gctggcttac agtttctctg tgtgctttgt ggttgctgtc atatttgctc |
| 4261 | tagaagaaaa aaaaaaaagg aggggaaatg cattttcccc agagataaag gctgccattt |
| 4321 | tgggggtctg tacttatggc ctgaaaatat ttgtgatcca taactctaca cagcctttac |
| 4381 | tcatactatt aggcacactt tccccttaga gccccctaag tttttcccag acgaatcttt |
| 4441 | ataatttctt tccaaagata ccaaataaac ttcagtgttt tcatctaatt ctcttaaagt |
| 4501 | tgatatctta atattttgtg ttgatcatta tttccattct taatgtgaaa aaaagtaatt |
| 4561 | atttatactt attataaaaa gtatttgaaa tttgcacatt taattgtccc taatagaaag |
| 4621 | ccacctattc tttgttggat ttcttcaagt ttttctaaat aaatgtaact tttcacaaga |
| 4681 | gtcaacatta aaaaataaat tatttaagaa cagaaaaaaa aaaaaaaaa |

The amino acid sequence of SEQ ID NO: 8 is shown below:

| | |
|---|---|
| 1 | maeqvlpqal ylsnmrkavk irertpedif kptngiihhf ktmhrytlem frtcqfcpqf |
| 61 | reiihkalid rniqatlesq kklnwcrevr klvalktngd gnclmhatsq ymwgvqdtdl |
| 121 | vlrkalfstl ketdtrnfkf rwqleslksq efvetglcyd trnwndewdn likmastdtp |
| 181 | marsglqyns leeihifvlc nilrrpiivi sdkmlrsles gsnfaplkvg giylplhwpa |
| 241 | qecyrypivl gydshhfvpl vtlkdsgpei ravplvnrdr grfedlkvhf ltdpenemke |
| 301 | kllkeylmvi eipvqgwdhg tthlinaakl deanlpkein lvddyfelvq heykkwqens |
| 361 | eqgrreghaq npmepsvpql slmdvkcetp ncpffmsvnt qplchecser rqknqnklpk |
| 421 | lnskpgpegl pgmalgasrg eayeplawnp eestggphsa pptapspflf settamkcrs |
| 481 | pgcpftlnvq hngfcerchn arqlhashap dhtrhldpgk cqaclqdvtr tfngicstcf |
| 541 | krttaeasss lstslppsch qrsksdpsrl vrspsphsch ragndapagc lsqaartpgd |
| 601 | rtgtskcrka gcvyfgtpen kgfcticfie yrenkhfaaa sgkvsptasr fqntipclgr |
| 661 | ecgtlgstmf egycqkcfie aqnqrfheak rteeqlrssq rrdvprttqs tsrpkcaras |
| 721 | cknilacrse elcmecqhpn qrmgpgahrg epapedppkq rcrapacdhf gnakcngycn |
| 781 | ecfqfkqmyg |

An A20 agonist is an agent that directly or indirectly stabilizes A20 polypeptide, e.g., partially or fully blocking or reducing degradation of A20 polypeptide or mRNA (which in turn increasing the half-life of A20 in brain endothelial cells); and/or directly or indirectly increases a biological activity of an A20 polypeptide (e.g., by increasing expression, localization, and/or activity of A20 polypeptide—e.g., an active form thereof). In some embodiments, A20 agonists may include, e.g., one or more of A20 agonistic antibodies (e.g., full length antibodies or antibody fragments, or agents that incorporate or include either), A20 polypeptide fragments or variants (e.g., polypeptide fragments or variants that retain a biological activity of native A20 polypeptides, whether produced, for example, by isolation, by chemical synthesis, by recombinant technologies, or otherwise) of native A20 polypeptides, A20 stimulatory small molecules, etc. Methods for identifying A20 agonists can comprise contacting a polypeptide with a candidate A20 agonist and measuring a detectable change in its level and/or one or more biological activities normally associated with an A20 polypeptide.

In some embodiments, an A20 agonist can be a molecule (e.g., an activating polynucleotide or oligonucleotide) that induces transcription and/or translation of A20 polypeptide, thereby increasing the mRNA/protein level of A20 polypeptide. In some embodiments, an A20 agonist described herein may increase the transcription and/or translation of A20 in brain endothelial cells (e.g., cerebral endothelial cells), for example by an amount that may be at least 20% or more, e.g., 30%, 40%, 50%, 60%, 70%, 80%, 90%, 95% or above, as compared to transcription and/or translation of A20 in brain endothelial cells in the absence of the A20 agonist. In some embodiments, an A20 agonist described herein may increase the transcription and/or translation of A20 in brain endothelial cells (e.g., cerebral endothelial cells), for example by an amount that may be at least 1.1-fold or more, e.g., 2-fold, 3-fold, 4-fold, 5-fold, or more, as compared to transcription and/or translation of A20 in brain endothelial cells in the absence of the A20 agonist. In some embodiments, activity of such an A20 agonist can be determined by a variety of methods, e.g., measuring transcript level and/or or protein level using any methods known in the art, e.g., mRNA expression assays such as PCR, and/or protein assays such as ELISA or Western blot.

In some embodiments, an A20 agonist may be or comprise an A20 polypeptide fragment or variant of a native A20 polypeptide that retains at least 30% or more (including, e.g., at least 40%, at least 50%, at least 60%, at least 70%, at least 80%, at least 90%, or more) of a biological activity of a native A20 polypeptide, whether produced, for example, by isolation, by chemical synthesis, by recombinant technologies, or otherwise.

In some embodiments, an A20 agonist may be or comprise an A20 peptidomimetic (e.g., peptoid).

In some embodiments, an A20 agonist may comprise an expression vector comprising nucleic acid agent that encodes an A20 or a functional fragment thereof.

Low Density Lipoprotein Receptor-Related Protein 1 (LRP1)

In some aspects, the present disclosure provides agents for increasing expression and/or activity of low density lipoprotein receptor-related protein 1 (LRP1). These agents are useful for inhibiting or reducing accumulation of AP, which can, in turn, provide therapeutic benefits for a neurovascular and/or neurodegenerative disease or disorder (e.g., Alzheimer's disease).

Low density lipoprotein receptor-related protein is a transmembrance glycoprotein that can be involved in intracellular signaling and endocytosis and is ubiquitously expressed in many tissues. It is known to be an endocytic receptor for many ligands, including, but not limited to, Aβ. Clearance of Aβ levels in cells, e.g. brain cells, can be impaired by decreasing f Lrp1 expression, possibly through inhibition of LRP1-mediated neuronal Aβ uptake and degradation.

An LRP1 agonist is an agent that directly or indirectly stabilizes or otherwise increases level of LRP1 polypeptide, e.g., at a particular point in time or over time, e.g., in some embodiments partially or fully blocking or reducing degradation of LRP1 polypeptide or mRNA (which in turn increasing the half-life of LRP1 in brain endothelial cells); and/or directly or indirectly increases a biological activity of a LRP1 polypeptide (e.g., by increasing expression, localization, and/or activity of LRP1 polypeptide—e.g., an active form thereof). In some embodiments, LRP1 agonists may include, e.g., one or more of LRP1 agonistic antibodies (e.g., full length antibodies or antibody fragments, or agents that incorporate or include either), LRP1 polypeptide fragments or variants (e.g., polypeptide fragments or variants that retain a biological activity of native LRP1 polypeptides, whether produced, for example, by isolation, by chemical synthesis, by recombinant technologies, or otherwise) of native LRP1 polypeptides, LRP1 stimulatory small molecules, etc.

In some embodiments a method for identifying or characterizing LRP1 agonists can comprise contacting an LRP1 polypeptide, or a system that comprises or expresses it, with a candidate LRP1 agonist and measuring a detectable change in its level and/or one or more biological activities normally associated with an LRP1 polypeptide.

In some embodiments, an LRP1 agonist can be a molecule (e.g., an activating polynucleotide or oligonucleotide) that induces or stimulates production (e.g., transcription and/or translation, and/or other processing or modification that generates or contributes to generation of an active LRP1 polypeptide) and/or level of LRP1 polypeptide, e.g., by increasing mRNA/protein/active form level of LRP1 polypeptide. In some embodiments, an LRP1 agonist described herein may increase level of LRP1 in brain endothelial cells (e.g., cerebral endothelial cells), for example by an amount that may be at least 20% or more, e.g., 30%, 40%, 50%, 60%, 70%, 80%, 90%, 95% or above, as compared to that observed in brain endothelial cells under comparable conditions lacking the LRP1 agonist. In some embodiments, an LRP1 agonist described herein may increase level of LRP1 in brain endothelial cells (e.g., cerebral endothelial cells), for example by an amount that may be at least 1.1-fold or more, e.g., 2-fold, 3-fold, 4-fold, 5-fold, or more, as compared to that observed in brain endothelial cells under comparable conditions lacking the LRP1 agonist.

In some embodiments, level and/or activity of an LRP1 agonist can be determined, for example, by measuring transcript level and/or or protein level using any methods known in the art, e.g., mRNA expression assays such as PCR, and/or protein assays such as ELISA or Western blot.

In some embodiments, an LRP1 agonist may be or comprise an LRP1 polypeptide fragment or variant of a native LRP1 polypeptide that retains at least 30% or more (including, e.g., at least 40%, at least 50%, at least 60%, at least 70%, at least 80%, at least 90%, or more) of a biological activity of a native LRP1 polypeptide, whether produced, for example, by isolation, by chemical synthesis, by recombinant technologies, or otherwise.

In some embodiments, an LRP1 agonist may be or comprise an LRP1 peptidomimetic (e.g., peptoid).

In some embodiments, an LRP1 agonist may be or comprise an expression vector comprising nucleic acid agent that encodes an LRP1 or a functional fragment thereof.

### Strategies for Brain Drug Delivery

Agents for inhibiting necroptosis, e.g., RIPK1 inhibitors, RIPK3 inhibitors, Nat1 or Nat2 agonists, and/or A20 agonists described herein are delivered to the brains of subjects. The present disclosure recognizes, among other things, inhibiting necroptosis in brain endothelial cells can provide therapeutic benefits for subjects suffering from or susceptible to neurovascular or neurodegenerative diseases (e.g., AD), including, e.g., reducing the severity of cognitive decline, and/or reducing neuroinflammation in the brains, e.g., of AD subjects. Accordingly, in some embodiments, an agent for inhibiting necroptosis (e.g., RIPK1 inhibitors, RIPK3 inhibitors, Nat1 or Nat2 agonists, and/or A20 agonists described herein) can be delivered to brain endothelial cells. In some embodiments, such a delivery method can be targeted to brain endothelial cells, e.g., cerebral endothelial cells, blood-brain barrier endothelial cells, or brain microvessels or microvasculature. In some embodiments, such a delivery method can be selectively targeted to brain endothelial cells, e.g., cerebral endothelial cells, blood-brain barrier endothelial cells, or brain microvessels or microvasculature.

Viral Vectors

In some embodiments, agents for inhibiting necroptosis, e.g., RIPK1 inhibitors, RIPK3 inhibitors, Nat1 or Nat2 agonists, and/or A20 agonists described herein, are delivered to the brains of subjects by use of viral vectors. Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. In some embodiments, vectors may not be incorporated into the cells' genome. The constructs can include viral sequences for transfection, if desired. Alternatively, the construct can be incorporated into vectors capable of episomal replication, e.g., EPV and EBV vectors.

In some embodiments, viral vectors that contain nucleic acid sequences encoding a target polypeptide (e.g., Nat1 or Nat2, or A20) or a nucleic acid agent (e.g., a nucleic acid agent that inhibits RIPK3 and/or RIPK1 such as an interfering RNA agent for RIPK3 or RIPK1) can be used. For example, a retroviral vector can be used. Retroviral vectors contain the components necessary for the correct packaging of the viral genome and integration into the host cell DNA. Examples of use of retroviral vectors in gene therapy include, but are not limited to: Lundstrom, Diseases 2018: 42 (2018); Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993). In some embodiments, lentiviral vectors may be used to deliver nucleic acid agents for inhibiting necroptosis (e.g., ones described herein). Examples of lentiviral vectors for use in the agents, compositions, and/or methods described herein include, for example, HIV-1 and HIV-2 based vectors, e.g., ones as described in U.S. Pat. Nos. 6,143,520; 5,665,557; and 5,981,276, which are herein incorporated by reference.

In some embodiments, adenoviruses can be used to deliver nucleic acid agents for inhibiting necroptosis (e.g., ones described herein). Adenoviruses have the advantage of being capable of infecting non-dividing cells. Kozarsky and Wilson, Current Opinion in Genetics and Development 3:499-503 (1993) present a review of adenovirus-based gene therapy. Examples of adenoviruses which can be used to deliver nucleic acid agents for inhibiting necroptosis (e.g., ones described herein) can be found, e.g., in Lee et al., Genes & Diseases 4:43-63 (2017).

In some embodiments, nucleic acid agents for inhibiting necroptosis (e.g., nucleic acid agent-based RIPK1 inhibitors, nucleic acid agent-based RIPK3 inhibitors, nucleic acid agent-based Nat1 or Nat2 agonists, and/or nucleic acid agent-based A20 agonists described herein) can be delivered by adeno-associated virus (AAV) vectors. In some embodiments, a AAV vector that expresses a nucleic acid agent for inhibiting necroptosis (e.g., nucleic acid agent-based RIPK1 inhibitors, nucleic acid agent-based RIPK3 inhibitors, nucleic acid agent-based Nat1 or Nat2 agonists, and/or nucleic acid agent-based A20 agonists described herein) is a recombinant AAV vector having, for example, either an U6 or H1 RNA promoter, or a cytomegalovirus (CMV) promoter. Suitable AAV vectors for use in agents, compositions, and methods described include, but are not limited to AAVs described in Passini et al., Methods Mol. Biol. 246: 225-36 (2004).

In some embodiments, a nucleic acid agent for inhibiting necroptosis (e.g., nucleic acid agent-based RIPK1 inhibitors, nucleic acid agent-based RIPK3 inhibitors, nucleic acid agent-based Nat1 or Nat2 agonists, and/or nucleic acid agent-based A20 agonists described herein) can be delivered by a brain endothelial cell-specific viral vector, e.g., an adeno-associated virus (AAV) vector. For example, in some embodiments, a brain endothelial cell-specific adeno-associated virus (AAV) vector may have a capsid sequence motif XXGXXWX (where X can be any amino acid). In some embodiments, a brain endothelial cell-specific adeno-associated virus (AAV) vector may have a capsid sequence motif SDGLAWV. In some embodiments, a brain endothelial cell-specific adeno-associated virus (AAV) vector may have a capsid sequence motif NRGTEWD. See Korbelin et al., EMBO Molecular Medicine 8(6): 609-625, which discloses a brain microvasculature endothelial cell-specific AAV vector. In some embodiments, brain endothelial cell-specific viral vectors, e.g., as described in WO 2015/158749, can be used to deliver nucleic acid agents for inhibiting necroptosis (e.g., nucleic acid agent-based RIPK1 inhibitors, nucleic acid agent-based RIPK3 inhibitors, nucleic acid agent-based Nat1 or Nat2 agonists, and/or nucleic acid agent-based A20 agonists described herein).

Non-Viral Nanoparticles

In some embodiments, an agent for inhibiting necroptosis, e.g., an RIPK1 inhibitor, an RIPK3 inhibitor, a Nat1 or Nat2 agonist, and/or an A20 agonist described herein may be conjugated to and/or encapsulated by a non-viral nanoparticle, e.g., to facilitate delivery of the agent to the brain of a subject. Non-viral nanoparticles may comprise one or more biocompatible polymers, e.g., PLGA. In some embodiments, a non-viral nanoparticle may be or comprise a lipoprotein. In some embodiments, a non-viral nanoparticle may comprise a surface ligand that enhances its transfer to target cells in the brain such as cerebral endothelial cells, blood-brain barrier endothelial cells, brain microvessels or microvasculature. In some embodiments, a surface ligand present in a non-viral nanoparticle may be a ligand for a transferrin receptor (e.g., transferrin). In some embodiments, a surface ligand present in a non-viral nanoparticle may be a ligand for insulin receptor. In some embodiments, a surface ligand present in a non-viral nanoparticle may be a ligand for glucose transporter (GLUT), e.g., GLUT1. In some embodiments a surface ligand present in a non-viral nanoparticle may be a ligand for low density lipoprotein (LDL) receptor-related protein (LRP), e.g., LRP1.

Brain-Targeting Antibodies or Ligands

In some embodiments, an agent for inhibiting necroptosis, e.g., an RIPK1 inhibitor, an RIPK3 inhibitor, a Nat1 or Nat2 agonist, and/or an A20 agonist described herein may be conjugated to a ligand (e.g., a small molecule, a peptide, polypeptide), or an antibody or fragment thereof that binds to a receptor for transcytosis that is selective and present in the blood-brain-barrier, e.g., to induce receptor-mediated transcytosis of the agent across the blood-brain-barrier. For example, transferrin receptor (TFRC) antibody chimeras have been successfully used in animal models of Parkinson's disease, stroke, Alzheimer's disease and lysosomal storage disease for enhancing brain accumulation of therapeutic proteins. Insulin receptor (INSR-targeted antibody and therapeutic protein fusions have been previously reported to show great potential in non-human primates for treating mucopolysaccharidosis type I (MPS-I) and Alzheimer's disease, and also in brain delivery of GDNF, TNFR, erythropoietin and paraoxonase-1. Accordingly, in some embodiments, an agent for inhibiting necroptosis, e.g., an RIPK1 inhibitor, an RIPK3 inhibitor, a Nat1 or Nat2 agonist, and/or an A20 agonist described herein may be conjugated to an antibody that binds to TFRC. Examples of TFRC antibodies that can be used herein include, but are not limited to antibodies described in Watts and Dennis, Curr. Opin. Chem. Biol. 17: 393-399 (2013); Pardridge, Expert Opin. Ther. Targets, 19 (8): 1059-1072 (2015); Lajoie and Shusta, Annu. Rev. Pharmacol. Toxicol., 55 (1): 613-631 (2015); and Niewoehner et al., Neuron, 81 (1): 49-60 (2014). In some embodiments, an agent for inhibiting necroptosis, e.g., an RIPK1 inhibitor, an RIPK3 inhibitor, a Nat1 or Nat2 agonist, and/or an A20 agonist described herein may be conjugated to an antibody that binds to INSR. An exemplary INSR antibody that can be used herein includes, but is not limited to antibodies described in Lajoie and Shusta, Annu. Rev. Pharmacol. Toxicol., 55 (1): 613-631 (2015). In some embodiments, an agent for inhibiting necroptosis, e.g., an RIPK1 inhibitor, an RIPK3 inhibitor, a Nat1 or Nat2 agonist, and/or an A20 agonist described herein may be conjugated to an antibody that binds to GLUT (e.g., GLUT-1), and/or LRP (e.g., LRP1). Exemplary transcytotic antibodies (e.g., against LRP) are described, e.g., in WO 2008/119096. Additional brain microvasculature selective receptors can be identified, e.g., using a proteomics- and transcriptomics-based assay, e.g., as described in Mager et al., Neuropharmacology 120: 4-7 (2017), and used in combination with agents for inhibiting necroptosis described herein.

Other Delivery Strategies

In some embodiments, an agent for inhibiting necroptosis, e.g., an RIPK1 inhibitor, an RIPK3 inhibitor, a Nat1 or Nat2 agonist, and/or an A20 agonist described herein may be delivered to the brain of a subject by use of an exosome. Methods of using exosomes to deliver therapeutic agents are described, e.g., in Ha et al. Acta Pharm Sin B. 6:287-96 (2016), and can be used to deliver agents and/or compositions described herein to inhibit necroptosis in the brain (e.g., brain endothelial cells) of a subject. Other strategies for brain drug delivery known in the art, e.g., as described in Dong, Theranostics 8: 1481-1493 (2018), can also be used to deliver an agent for inhibiting necroptosis, e.g., an RIPK1 inhibitor, an RIPK3 inhibitor, a Nat1 or Nat2 agonist, and/or an A20 agonist described herein to the brain (e.g., brain endothelial cells) of a subject. In some embodiments, gene editing in brain endothelial cells using a promoter that drives brain endothelial cell-specific expression of genetic modulators (e.g., Nat1 or Nat2 and/or A20) can be used, e.g., as described in Assmann et al., Biochimica et Biophysica Acta (BBA)—Molecular Basis of Disease, 1862: 381-394 (2016). Examples of brain endothelial cell-specific promoters include, but are not limited to Alk1, VE-cadherin (Cdh5), Flk-1, Mx1, Pdgfb, Slco1c1, Sftpa1, Tal1, Tie1 or Tie2, VWF, and CD31.

### Pharmaceutical Compositions and Administration

In some embodiments, at least one or more agents (e.g., at least one, at least two or more agents) for inhibiting necroptosis (e.g., an RIPK3 inhibitor, an RIPK1 inhibitor, a Nat2 or Nat1 agonist, and/or an A20 agonist described herein) can be included in a pharmaceutical composition.

In some embodiments, a pharmaceutical composition can include a pharmaceutically acceptable carrier or excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Suitable pharmaceutically acceptable carriers include but are not limited to water, salt solutions (e.g., NaCl), saline, buffered saline, glycerol, sugars such as mannitol, sucrose, or others, dextrose, fatty acid esters, etc., as well as combinations thereof.

A pharmaceutical composition can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interfere with their activity. In certain embodiments, a water-soluble carrier suitable for intravenous administration is used. In some embodiments, a pharmaceutical composition can be sterile.

A suitable pharmaceutical composition, if desired, can also contain minor amounts of wetting or emulsifying agents, or pH buffering agents. A pharmaceutical composition can be a liquid solution, suspension, or emulsion.

A pharmaceutical composition can be formulated in accordance with the routine procedures as a pharmaceutical composition adapted for administration to human beings. The formulation of a pharmaceutical composition should suit the mode of administration. For example, in some embodiments, a composition for intravenous administration is typically a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where a pharmaceutical composition is to be administered by infusion, it can be dispensed with an infusion bottle containing sterile pharmaceutical grade water, saline or dextrose/water. Where a pharmaceutical composition is administered by injection, an ampule of sterile water for injection or saline can be provided so that the ingredients may be mixed prior to administration.

In some embodiments, pharmaceutical compositions provided herein may be administered orally, parenterally, by inhalation spray, nasally, intrathecally, intraperitoneally, intracisternally, intraventricularly, or via an implanted reservoir. In some embodiments, pharmaceutical compositions provided herein are administered orally, intraperitoneally or intravenously. In some embodiments, pharmaceutical compositions provided herein are administered intranasally. In some embodiments, pharmaceutical compositions provided herein are administered intracisternally. In some embodiments, pharmaceutical compositions provided herein are administered intraventricularly. In some embodiments, pharmaceutical compositions provided herein are administered by injection.

Sterile injectable forms of pharmaceutical compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In some embodiments, pharmaceutical compositions described herein may be formulated for delayed absorption. For example, delayed absorption of a parenteral formulation can be accomplished by dissolving or suspending, e.g., an agent for inhibiting necroptosis (e.g., ones described herein), in an oil vehicle. Alternatively, injectable depot forms are made by forming microcapsule matrices in biodegradable polymers such as polylactide-polyglycolide. Depending upon the ratio of active pharmaceutical ingredient (API) (e.g., agents for inhibiting necroptosis described herein) to polymer and the nature of the particular polymer employed, the rate of API release can be controlled. Examples of other biodegradable polymers include polyesters, poly(orthoesters) and poly(anhydrides). Depot injectable formulations are also prepared by entrapping one or more APIs in liposomes or microemulsions that are compatible with body tissues.

In some embodiments, pharmaceutical compositions provided herein are formulated for oral administration. Such pharmaceutical compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, one or more APIs are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and/or i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. One or more APIs (e.g., agents for inhibiting necroptosis described herein) can also be in micro-encapsulated form with one or more excipients as noted above. The solid dosage forms of tablets, dragees, capsules, pills, and granules can be prepared with coatings and shells such as enteric coatings, release control coatings, and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Pharmaceutical compositions provided herein may also be locally administered to a target site in the brain of a subject. Suitable topical formulations are readily prepared for each of these areas or organs. For example, for topical applications, pharmaceutical compositions may be formulated in a suitable ointment containing the active component suspended or dissolved in one or more carriers. Carriers for topical administration may include, but are not limited to, mineral oil, liquid petrolatum, white petrolatum, propylene glycol, polyoxyethylene, polyoxypropylene compound, emulsifying wax and water. Alternatively, pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts or cells in vitro or ex vivo. Modification of pharmaceutical compositions suitable for administration to humans in order to render the compositions suitable for administration to various animals or cells in vitro or ex vivo is well understood, and the ordinarily skilled practitioner, e.g., a veterinary pharmacologist, can design and/or perform such modification with merely ordinary, if any, experimentation.

Formulations of the pharmaceutical compositions described herein may be prepared by any method known or hereafter developed in the art of pharmacology. In general, such preparatory methods include the step of bringing the active ingredient into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of a pharmaceutical composition described herein. For example, a unit dose of a pharmaceutical composition comprises a predetermined amount of at least one or more agents for inhibiting necroptosis described herein (e.g., at least one or more RIPK3 inhibitors, at least one or more RIPK1 inhibitors, at least one or more Nat1 or Nat2 agonists, at least one or more A20 agonists described herein).

Relative amounts of any components in pharmaceutical compositions described herein, e.g., at least one or more agents for inhibiting necroptosis described herein (e.g., at least one or more RIPK3 inhibitors, at least one or more RIPK1 inhibitors, at least one or more Nat1 or Nat2 agonists, at least one or more A20 agonists described herein), a pharmaceutically acceptable excipient, and/or any additional ingredients can vary, depending upon the subject to be treated, target cells, and may also further depend upon the route by which the composition is to be administered.

### Kits

Another aspect of the present disclosure further provides a pharmaceutical pack or kit comprising one or more containers filled with at least one or more agents for inhibiting necroptosis described herein (e.g., at least one or more RIPK3 inhibitors, at least one or more RIPK1 inhibitors, at least one or more Nat1 or Nat2 agonists, at least one or more A20 agonists described herein). Kits may be used in any applicable method, including, for example, cell treatment, therapeutically or diagnostically. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

In some embodiments, kits provided herein may further comprise a delivery device, e.g., an injection device such a syringe and/or a catheter.

### Methods of Uses

The present disclosure, in some aspects, provides methods by which necroptosis in brains of subject in need thereof is reduced, thereby providing therapeutic benefits, e.g., reducing neuroinflammation mediated by necroptosis, reducing the severity of cognitive decline associated with necroptosis, and/or improving the cognitive function of a subject suffering from necroptosis in the brain (e.g., necroptosis in brain endothelial cells, blood-brain-barrier endothelial cells, microvessels and/or microvasculature). These methods can be also useful for treating necroptosis-mediated neurovascular or neurodegenerative diseases or disorders.

In some embodiments, agents, compositions, and methods may be used to treat any of the following disorders where necroptosis is likely to play a substantial role in the brain (e.g., necroptosis in brain endothelial cells such as cerebral endothelial cells, blood-brain-barrier endothelial cells, brain microvessels, or brain microvasculature), including, but are not limited to neurovascular or neurodegenerative disease associated with the brain or central or peripheral nervous system. Exemplary neurovascular or neurodegenerative diseases include, but are not limited to Alzheimer's disease, stroke, Huntington's disease, Parkinson's disease, amyotrophic lateral sclerosis, HIV associated dementia, cerebral ischemia, amyotrophic lateral sclerosis, multiple sclerosis, Lewy body disease, Menke's disease, Wilson's disease, Creutzfeldt Jakob disease, and Fahr disease.

In some embodiments, agents, compositions, and methods may be used to treat Alzheimer's disease (AD). Alzheimer's disease (AD) is a multifactorial disease with majority of demented patients displaying cerebrovascular pathology (Iadecola, C., Neuron. 80(4): 844-66 (2013)). It has been suggested that cerebrovascular dysregulation may contribute to the neurodegeneration in AD by promoting the dysfunction of endothelial cells (ECs) that comprise the blood-brain barrier (BBB), a key mediator of cerebral homeostasis (Di Marco, L. Y., et al., Neurobiol Dis. 82: 593-606 (2015); Iturria-Medina, Y., et al., Nat Commun. 7: 11934 (016)). However, the mechanism that mediates the cerebrovascular endothelial cell death in AD was unclear. It was also unclear whether the prevention of brain EC death and dysfunction may provide therapeutic benefits for AD.

The brain ECs display an arteriovenous hierarchy characterized by distinctive morphological, molecular and functional features (Vanlandewijck, M., et al., A molecular atlas of cell types and zonation in the brain vasculature. Nature, 2018. 554(7693): p. 475-480). Arteriolar, capillary and venular dysfunctions may be differentially affected by AD pathology. E.g. accelerated retinal venular attenuation compared to that of arterioles was found in AD patients (Frost, S., et al., Transl Psychiatry 3: e233 (2013)). In addition, cerebral amyloid angiopathy (CAA) was found to correlate with BBB permeability in capillaries while arterial endothelial basement membrane was relatively spared from amyloid deposits (Magaki, S., et al., Neurobiol Aging 70: 70-77 (2018)). However, the pathological mechanism that mediates the differential impairment of venular vs. arteriolar endothelial cells in AD was unclear. Understanding the mechanism of endothelial heterogeneity in AD pathogenesis and developing strategies to ameliorate vascular pathology are some of the major challenges in developing treatment for AD.

The recent development of single cell RNA sequencing has enabled the application of large-scale genomic approaches to determine gene expression patterns with single cell resolution (Klein, A. M., et al., Cell 161(5): 1187-1201 (2015); Macosko, E. Z., et al., Cell 161(5): 1202-1214 (2015)), which provides an unprecedented opportunity to understand the complexity of ECs with AD progresses. Using this technology, the present disclosure provides the recognition that venular and capillary ECs express higher levels of RIPK1 than that of arteriolar ECs and are selectively vulnerable to necroptosis in AD. Inhibition of necroptosis by blocking RIPK1 kinase using D138N knock in mutation or selective blocking necroptosis of brain ECs using AAV virus mediated RIPK3 knockdown blocks the BBB impairment and rescues cognitive deficits in AD mice. The present disclosure demonstrates the importance of RIPK1 and RIPK3 dependent EC dysfunction in driving AD pathogenesis and suggest that potential therapeutic benefit of blocking necroptosis of brain ECs for the treatment of AD. Further, the present disclosure demonstrates that the expression of N-acetyltransferase 1 (Nat1) in cerebral endothelial cells is reduced in an AD mouse model and AD patients. Murine Nat1 ortholog in human is Nat2, which has been identified as a risk factor for developing insulin resistance and diabetes. The present disclosure also demonstrates that the loss of Nat1 leads to the degradation of tumor necrosis factor alpha-induced protein 3 (Tnfaip3/A20), a negative regulator of RIPK1 activation and sensitizes endothelial cells to TNF-alpha induced cell death. Additionally, the present disclosure demonstrates that upregulation of Nat1 via cerebral endothelial cell-specific adeno-associated virus (AAV) successfully rescues cognitive declines in AD mice. Therefore, Nat2 in humans can be a novel molecular target to protect the integrity of cerebral endothelial cells and the blood-brain-barrier for treatment of AD.

Accordingly, methods for using any embodiment of agents for inhibiting necroptosis, and pharmaceutical compositions are provided herein. In some embodiments, a method of treating a neurovascular or neurodegenerative disease or disorder in a subject comprises inhibiting or reducing necroptosis in brain endothelial cells of the subject. In some embodiments, the step of inhibiting necroptosis comprises performing at least one of the following steps:
a. inhibiting expression and/or activity of receptor-interacting serine/threonine-protein kinase 3 (RIPK3) in brain endothelial cells of a subject in need thereof;
b. inhibiting expression and/or activity of receptor-interacting serine/threonine-protein kinase 1 (RIPK1) in brain endothelial cells of a subject in need thereof;
c. increasing expression and/or activity of N-acetyltransferase 1 (Nat1) or N-acetyltransferase 2 (Nat2) in brain endothelial cells of a subject in need thereof; and
d. increasing expression and/or activity of tumor necrosis factor alpha-induced protein 3 (Tnfaip3/A20) in brain endothelial cells of a subject in need thereof.

In some embodiments, a method described herein comprises inhibiting or reducing expression and/or activity of RIPK3 in brain endothelial cells of a subject in need thereof by at least 20%, including, e.g., at least 30%, at least 40%, at least 50%, at least 60%, at least 70%, at least 80%, at least 90%, at least 95%, at least 98%, at least 99%, or more, as compared to a reference level of expression and/or activity of RIPK3. In some embodiments, expression and/or activity of RIPK3 in brain endothelial cells is inhibited by administering one or more RIPK3 inhibitors (e.g., ones described herein). In some embodiments, one or more RIPK3 inhibitors to be administered are configured to be delivered to brain endothelial cells (e.g., cerebral endothelial cells, blood-brain-barrier endothelial cells, brain microvessels or brain microvasculature) using one or more brain delivery strategies described herein. In some embodiments, one or more RIPK3 inhibitors to be administered are configured to be selectively delivered to brain endothelial cells (e.g., cerebral endothelial cells, blood-brain-barrier endothelial cells, brain microvessels or brain microvasculature) using one or more brain delivery strategies described herein. In some embodiments, an RIPK3 inhibitor used in methods described herein is or comprises a brain endothelial cell-specific viral vector (e.g., adeno-associated viral vector) that expresses a nucleic acid agent that inhibits expression of RIPK3. In some embodiments, a nucleic acid agent of an RIPK3 inhibitor is or comprises an RNA agent whose nucleotide sequence is sufficiently complementary to that of an RIPK3 transcript, or portion thereof, that the nucleic acid agent hybridizes to the RIPK3 transcript. In some embodiments, when a nucleic acid agent of an RIPK3 inhibitor is present, RIPK3 level, RIPK3 transcript level, or both, is reduced (e.g., by at least 20%, including, e.g., at least 30%, at least 40%, at least 50%, at least 60%, at least 70%, at least 80%, at least 90%, at least 95%, at least 98%, at least 99%, or more), as compared with otherwise comparable conditions when the nucleic acid agent is absent. In some embodiments, a nucleic acid agent of an RIPK3 inhibitor is or comprises a short hairpin RNA (shRNA).

In some embodiments, a method described herein comprises inhibiting or reducing expression and/or activity of RIPK1 in brain endothelial cells of a subject in need thereof by at least 20%, including, e.g., at least 30%, at least 40%, at least 50%, at least 60%, at least 70%, at least 80%, at least 90%, at least 95%, at least 98%, at least 99%, or more, as compared to a reference level of expression and/or activity of RIPK1. In some embodiments, expression and/or activity of RIPK1 in brain endothelial cells is inhibited by administering one or more RIPK1 inhibitors (e.g., ones described herein). In some embodiments, one or more RIPK1 inhibitors to be administered are configured to be delivered to brain endothelial cells (e.g., cerebral endothelial cells, blood-brain-barrier endothelial cells, brain microvessels or brain microvasculature) using one or more brain delivery strategies described herein. In some embodiments, one or more RIPK1 inhibitors to be administered are configured to be selectively delivered to brain endothelial cells (e.g., cerebral endothelial cells, blood-brain-barrier endothelial cells, brain microvessels or brain microvasculature) using one or more brain delivery strategies described herein. In some embodiments, an RIPK1 inhibitor used in methods described herein is or comprises a brain endothelial cell-specific viral vector (e.g., adeno-associated viral vector) that expresses a nucleic acid agent that inhibits expression of RIPK1. In some embodiments, a nucleic acid agent of an RIPK1 inhibitor is or comprises an RNA agent whose nucleotide sequence is sufficiently complementary to that of an RIPK1 transcript, or portion thereof, that the nucleic acid agent hybridizes to the RIPK1 transcript. In some embodiments, when a nucleic acid agent of an RIPK1 inhibitor is present, RIPK1 level, RIPK1 transcript level, or both, is reduced (e.g., by at least 20%, including, e.g., at least 30%, at least 40%, at least 50%, at least 60%, at least 70%, at least 80%, at least 90%, at least 95%, at least 98%, at least 99%, or more), as compared with otherwise comparable conditions when the nucleic acid agent is absent. In some embodiments, a nucleic acid agent of an RIPK1 inhibitor is or comprises a short hairpin RNA (shRNA).

In some embodiments, a method described herein comprises increasing expression and/or activity of Nat1 or Nat2 in brain endothelial cells of a subject in need thereof by at least 20%, including, e.g., at least 30%, at least 40%, at least 50%, at least 60%, at least 70%, at least 80%, at least 90%, at least 95%, at least 98%, at least 99%, or more, as compared to a reference level of expression and/or activity of Nat1 or Nat2. In some embodiments, a method described herein comprises increasing expression and/or activity of Nat1 or Nat2 in brain endothelial cells of a subject in need thereof by at least 1.1-fold, including, e.g., at least 1.5-fold, at least 2-fold, at least 3-fold, at least 4-fold, at least 5-fold, at least 6-fold, at least 7-fold, at least 8-fold, at least 9-fold, at least 10-fold, or more, as compared to a reference level of expression and/or activity of Nat1 or Nat2. In some embodiments, expression and/or activity of Nat1 or Nat2 in brain endothelial cells is increased by administering one or more Nat1 or Nat2 agonists (e.g., ones described herein). In some embodiments, one or more Nat1 or Nat2 agonists to be administered are configured to be delivered to brain endothelial cells (e.g., cerebral endothelial cells, blood-brain-barrier endothelial cells, brain microvessels or brain microvasculature) using one or more brain delivery strategies described herein. In some embodiments, one or more Nat1 or Nat2 agonists to be administered are configured to be selectively delivered to brain endothelial cells (e.g., cerebral endothelial cells, blood-brain-barrier endothelial cells, brain microvessels or brain microvasculature) using one or more brain delivery strategies described herein. In some embodiments, a Nat1 or Nat2 agonist used in methods described herein is or comprises a brain endothelial cell-specific viral vector (e.g., adeno-associated viral vector) comprising a nucleic acid agent that encodes Nat1 or Nat2, or a functional fragment thereof. In some embodiments, when a nucleic acid agent of a Nat1 or Nat2 agonist is present, Nat1 or Nat2 level, Nat1 or Nat2 transcript level, or both, is increased (e.g., by at least 20%, including, e.g., at least 30%, at least 40%, at least 50%, at least 60%, at least 70%, at least 80%, at least 90%, at least 95%, at least 98%, at least 99%, or more), as compared with otherwise comparable conditions when the nucleic acid agent is absent. In some embodiments, when a nucleic acid agent of a Nat1 or Nat2 agonist is present, Nat1 or Nat2 level, Nat1 or Nat2 transcript level, or both, is increased (e.g., by at least 1.1-fold, including, e.g., at least 1.5-fold, at least 2-fold, at least 3-fold, at least 4-fold, at least 5-fold, at least 6-fold, at least 7-fold, at least 8-fold, at least 9-fold, at least 10-fold, or more), as compared with otherwise comparable conditions when the nucleic acid agent is absent. In some embodiments, when a subject to be treated is a mouse subject, a Nat1 agonist is administered to the subject. In some embodiments, when a subject to be treated is a human subject, a Nat2 agonist is administered to the subject.

In some embodiments, a method described herein comprises increasing expression and/or activity of Tnfaip3/A20 in brain endothelial cells of a subject in need thereof by at least 20%, including, e.g., at least 30%, at least 40%, at least 50%, at least 60%, at least 70%, at least 80%, at least 90%, at least 95%, at least 98%, at least 99%, or more, as compared to a reference level of expression and/or activity of Tnfaip3/A20. In some embodiments, a method described herein comprises increasing expression and/or activity of Tnfaip3/A20 in brain endothelial cells of a subject in need thereof by at least 1.1-fold, including, e.g., at least 1.5-fold, at least 2-fold, at least 3-fold, at least 4-fold, at least 5-fold, at least 6-fold, at least 7-fold, at least 8-fold, at least 9-fold, at least 10-fold, or more, as compared to a reference level of expression and/or activity of Tnfaip3/A20. In some embodiments, expression and/or activity of Tnfaip3/A20 in brain endothelial cells is increased by administering one or more A20 agonists (e.g., ones described herein). In some embodiments, one or more A20 agonists to be administered are configured to be delivered to brain endothelial cells (e.g., cerebral endothelial cells, blood-brain-barrier endothelial cells, brain microvessels or brain microvasculature) using one or more brain delivery strategies described herein. In some embodiments, one or more A20 agonists to be administered are configured to be selectively delivered to brain endothelial cells (e.g., cerebral endothelial cells, blood-brain-barrier endothelial cells, brain microvessels or brain microvasculature) using one or more brain delivery strategies described herein. In some embodiments, an A20 agonist used in methods described herein is or comprises a brain endothelial cell-specific viral vector (e.g., adeno-associated viral vector) comprising a nucleic acid agent that encodes Tnfaip3/A20, or a functional fragment thereof. In some embodiments, when a nucleic acid agent of an A20 agonist is present, Tnfaip3/A20 level, Tnfaip3/A20 transcript level, or both, is increased (e.g., by at least 20%, including, e.g., at least 30%, at least 40%, at least 50%, at least 60%, at least 70%, at least 80%, at least 90%, at least 95%, at least 98%, at least 99%, or more), as compared with otherwise comparable conditions when the nucleic acid agent is absent. In some embodiments, when a nucleic acid agent of an A20 agonist is present, Tnfaip3/A20 level, Tnfaip3/A20 transcript level, or both, is increased (e.g., by at least 1.1-fold, including, e.g., at least 1.5-fold, at least 2-fold, at least 3-fold, at least 4-fold, at least 5-fold, at least 6-fold, at least 7-fold, at least 8-fold, at least 9-fold, at least 10-fold, or more), as compared with otherwise comparable conditions when the nucleic acid agent is absent.

In some embodiments, a method described herein comprises increasing level and/or activity of LRP1 in brain endothelial cells of a subject in need thereof by at least 20%, including, e.g., at least 30%, at least 40%, at least 50%, at least 60%, at least 70%, at least 80%, at least 90%, at least 95%, at least 98%, at least 99%, or more, as compared to a reference level and/or activity of LRP1.

In some embodiments, a method described herein comprises increasing level and/or activity of LRP1 in brain endothelial cells of a subject in need thereof by at least 1.1-fold, including, e.g., at least 1.5-fold, at least 2-fold, at least 3-fold, at least 4-fold, at least 5-fold, at least 6-fold, at least 7-fold, at least 8-fold, at least 9-fold, at least 10-fold, or more, as compared to a reference level and/or activity of LRP1. In some embodiments, level and/or activity of LRP1 in brain endothelial cells is increased by administering one or more LRP1 agonists (e.g., ones described herein). In some embodiments, one or more LRP1 agonists to be administered are configured to be delivered (e.g., are formulated for delivery) to brain endothelial cells (e.g., cerebral endothelial cells, blood-brain-barrier endothelial cells, brain microvessels or brain microvasculature) using one or more brain delivery strategies described herein. In some embodiments, one or more LRP1 agonists to be administered are configured to be selectively delivered to brain endothelial cells (e.g., cerebral endothelial cells, blood-brain-barrier endothelial cells, brain microvessels or brain microvasculature) using one or more brain delivery strategies described herein. In some embodiments, an LRP1 agonist used in methods described herein is or comprises a brain endothelial cell-specific viral vector (e.g., adeno-associated viral vector) comprising a nucleic acid agent that encodes LRP1, or a functional fragment thereof. In some embodiments, when a nucleic acid agent of an LRP1 agonist is present, LRP1 level, LRP1 transcript level, or both, is increased (e.g., by at least 20%, including, e.g., at least 30%, at least 40%, at least 50%, at least 60%, at least 70%, at least 80%, at least 90%, at least 95%, at least 98%, at least 99%, or more), as compared with otherwise comparable conditions when the nucleic acid agent is absent. In some embodiments, when a nucleic acid agent of an LRP1 agonist is present, LRP1 level, LRP1 transcript level, or both, is increased (e.g., by at least 1.1-fold, including, e.g., at least 1.5-fold, at least 2-fold, at least 3-fold, at least 4-fold, at least 5-fold, at least 6-fold, at least 7-fold, at least 8-fold, at least 9-fold, at least 10-fold, or more), as compared with otherwise comparable conditions when the nucleic acid agent is absent.

In some embodiments involving any methods described herein, a subject to be treated may be suffering from or susceptible to neuroinflammation in the subject's brain; and necroptosis is inhibited by administering an agent for inhibiting necroptosis (e.g., ones described herein) to an extent sufficient that the neuroinflammation is reduced, e.g., by at least 20%, including, e.g., at least 30%, at least 40%, at least 50%, at least 60%, at least 70%, at least 80%, at least 90%, at least 95%, at least 98%, at least 99%, or more), as compared with otherwise comparable conditions when the agent for inhibiting necroptosis is absent. In some embodiments, level of the neuroinflammation can be assessed by detecting level of interferon or interleukin-1β or both.

Another aspect provided herein is a method of inhibiting necroptosis in the brain of a subject in need thereof, which involves increasing expression and/or activity of N-acetyltransferase 1 (Nat1) or N-acetyltransferase 2 (Nat2) in the brain of a subject. In some embodiments, expression and/or activity of Nat1 or Nat2 in brain endothelial cells of a subject in need thereof is increased by at least 20%, including, e.g., at least 30%, at least 40%, at least 50%, at least 60%, at least 70%, at least 80%, at least 90%, at least 95%, at least 98%, at least 99%, or more, as compared to a reference level of expression and/or activity of Nat1 or Nat2. In some embodiments, expression and/or activity of Nat1 or Nat2 in brain endothelial cells of a subject in need thereof is increased by at least 1.1-fold, including, e.g., at least 1.5-fold, at least 2-fold, at least 3-fold, at least 4-fold, at least 5-fold, at least 6-fold, at least 7-fold, at least 8-fold, at least 9-fold, at least 10-fold, or more, as compared to a reference level of expression and/or activity of Nat1 or Nat2.

In some embodiments, expression and/or activity of Nat1 or Nat2 is increased to an extent sufficient that RIPK1 activation in the brain of a subject to be treated is inhibited. For example, in some embodiments, expression and/or activity of Nat1 or Nat2 is increased such that RIPK1 activation is inhibited by at least 10%, including, e.g., at least 20%, at least 30%, at least 40%, at least 50%, at least 60%, at least 70%, at least 80%, at least 90%, at least 95%, or more, as compared with otherwise comparable conditions when increased Nat1 or Nat2 expression and/or activity is absent.

In some embodiments, expression and/or activity of Nat1 or Nat2 is increased to an extent sufficient that level of tumor necrosis factor alpha-induced protein 3 (Tnfaip3/A20) in the brain of a subject to be treated is increased. For example, in some embodiments, expression and/or activity of Nat1 or Nat2 is increased such that level of Tnfaip3/A30 is increased by at least 10%, including, e.g., at least 20%, at least 30%, at least 40%, at least 50%, at least 60%, at least 70%, at least 80%, at least 90%, at least 95%, or more, as compared with otherwise comparable conditions when increased Nat1 or Nat2 expression and/or activity is absent. In some embodiments, expression and/or activity of Nat1 or Nat2 is increased such that level of Tnfaip3/A30 is increased by at least 1.1-fold, including, e.g., at least 1.5-fold, at least 2-fold, at least 3-fold, at least 4-fold, at least 5-fold, at least 6-fold, at least 7-fold, at least 8-fold, at least 9-fold, at least 10-fold, or more, as compared with otherwise comparable conditions when increased Nat1 or Nat2 expression and/or activity is absent.

In some embodiments, expression and/or activity of Nat1 or Nat2 in brain endothelial cells is increased by administering one or more Nat1 or Nat2 agonists (e.g., ones described herein). In some embodiments, one or more Nat1 or Nat2 agonists to be administered are configured to be delivered to brain endothelial cells (e.g., cerebral endothelial cells, blood-brain-barrier endothelial cells, brain microvessels or brain microvasculature) using one or more brain delivery strategies described herein. In some embodiments, one or more Nat1 or Nat2 agonists to be administered are configured to be selectively delivered to brain endothelial cells (e.g., cerebral endothelial cells, blood-brain-barrier endothelial cells, brain microvessels or brain microvasculature) using one or more brain delivery strategies described herein. In some embodiments, a Nat1 or Nat2 agonist used in methods described herein is or comprises a brain endothelial cell-specific viral vector (e.g., adeno-associated viral vector) comprising a nucleic acid agent that encodes Nat1 or Nat2, or a functional fragment thereof. In some embodiments, when a nucleic acid agent of a Nat1 or Nat2 agonist is present, Nat1 or Nat2 level, Nat1 or Nat2 transcript level, or both, is increased (e.g., by at least 20%, including, e.g., at least 30%, at least 40%, at least 50%, at least 60%, at least 70%, at least 80%, at least 90%, at least 95%, at least 98%, at least 99%, or more), as compared with otherwise comparable conditions when the nucleic acid agent is absent. In some embodiments, when a nucleic acid agent of a Nat1 or Nat2 agonist is present, Nat1 or Nat2 level, Nat1 or Nat2 transcript level, or both, is increased (e.g., by at least 1.1-fold, including, e.g., at least 1.5-fold, at least 2-fold, at least 3-fold, at least 4-fold, at least 5-fold, at least 6-fold, at least 7-fold, at least 8-fold, at least 9-fold, at least 10-fold, or more), as compared with otherwise comparable conditions when the nucleic acid agent is absent. In some embodiments, when a subject to be treated is a mouse subject, a Nat1 agonist is administered to the subject. In some embodiments, when a subject to be treated is a human subject, a Nat2 agonist is administered to the subject. In some embodiments, a subject to be treated is suffering from or susceptible to a necroptosis-mediated neurovascular disease or disorder, e.g., Alzheimer's disease.

Other features of the invention will become apparent in the course of the following descriptions of exemplary embodiments, which are given for illustration of the invention and are not intended to be limiting thereof.

### Exemplification

### Example 1: Selective Vulnerability of Venular and Capillary ECs in AD Brain

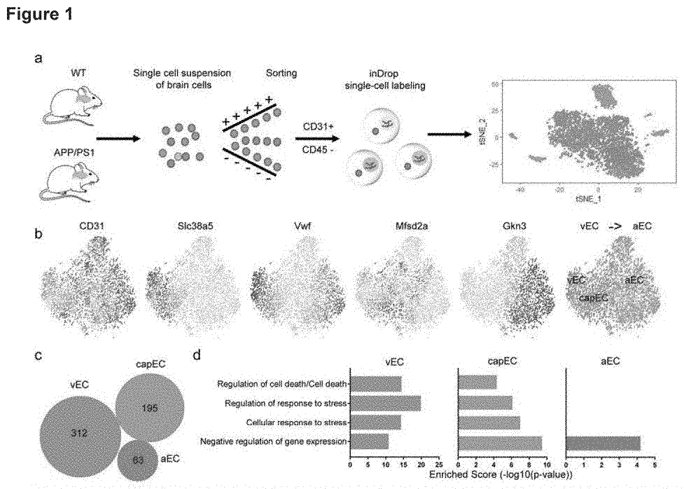

To understand the complexity of endothelial cells (ECs) in the pathogenesis of AD, fluorescence-activated cell sorting (FACS) as used to isolate ECs from the cerebrum of wild-type (WT) and APPswePSEN1dE9(APP/PS1) mice (5-6 months of age) (FIG. 1, panel a). Single cell RNA sequencing was completed with inDrop (Klein, A. M., et al. Cell, 161(5): 1187-120 (2015)) and Seurat was used to generate t-SNE plot with 4538 single cells that were separated into 10 clusters (FIG. 1, panel a). Among these clusters, non-ECs, including microglia, smooth muscle cells (SMC) and stromal cells, were detected based on their molecular signatures (Vanlandewijck, M. et al. Nature 554, 475-580 (2018)) and excluded for further analysis. The sequenced populations were confirmed that were comprised of ECs by high RNA expression of CD31 (FIG. 1, panel b). Unbiased clustering of ECs with Seurat identified three subpopulations of ECs. The differential expressed genes between these three clusters have greatly overlapped with endothelial arteriovenous markers published in Vanlandewijck, M. et al. Nature 554, 475-580 (2018). These clusters were then able to be confirmed as venous endothelial cell (vEC, Slc38a5⁺Vwf⁺Gkn3⁺), capillary endothelial cell (capEC, Vwf⁻Mfsd2a⁺) and arterial endothelial cell (aEC, Slc38a5⁻Vwf⁺Gkn3⁺) (FIG. 1, panel b). Subsequent analysis of single cell transcriptome expressions by SCDE (Kharchenko et al. Nat Methods 11: 740-2 (2014)) has identified 312 genes, 195 genes and 63 genes differently expressed between WT and APP/PS mice in vEC, capEC and aEC, separately (FIG. 1, panel c). This result is quite surprising as the cell number of aECs (2281 cells) captured for sequencing more than sextuples that of vECs (362 cells) and theoretically more significant differential genes should be detected in aEC if AD pathogenesis has equal impacts on these EC subpopulations. This result indicates that vECs and capECs may be selectively more vulnerable to the pathogenesis of AD compared to aECs. GO gene set analysis of these differential expressed genes has revealed that multiple common signaling pathways between vECs and capECs, but not in aECs, are disturbed during AD pathogenesis, including the one that regulates cell death (FIG. 1, panel d)

### Example 2: Selective Loss of Venular and Capillary ECs in AD Brain

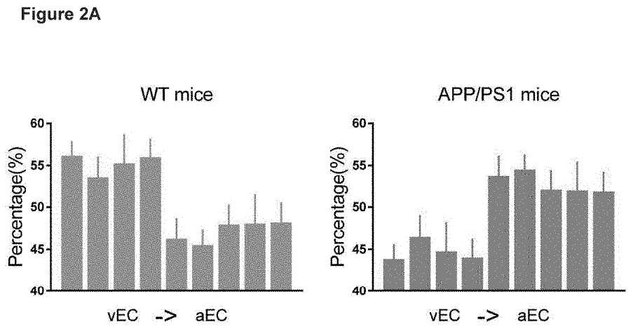

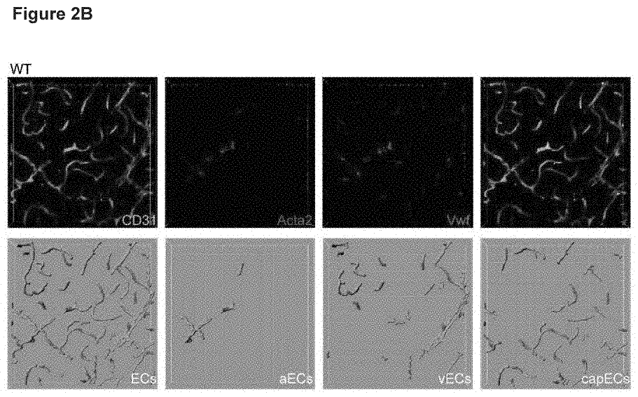

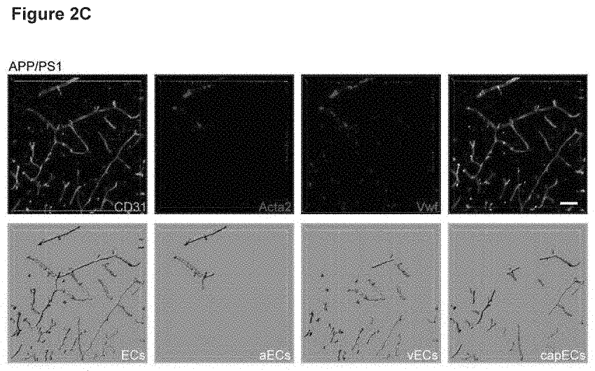

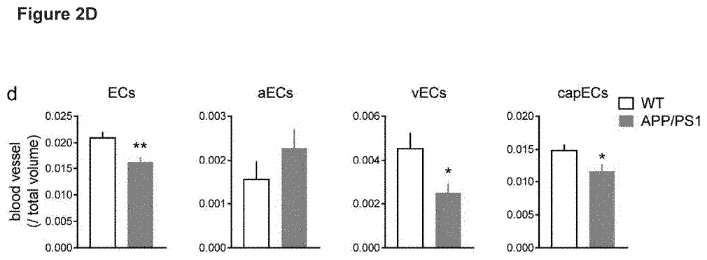

Enlightened by the finding that cell death pathway in vECs and capECs correlates with AD pathogenesis, the proportions of ECs in WT or APP/PS1 mice were calculated along venous to arteriole axis (FIG. 2A). In WT mice, the EC proportion decreases along this axis, while it increases in APP/PS1 mice. To figure out the reason that causes the change of EC subtype prevalence, aECs, vECs and capECs were identified by immunochemistry (as described in Vanlandewijck, M. et al. Nature 554, 475-580 (2018)) (FIGS. 2B and 2C). ECs were detected with anti-CD31 antibody and aECs were confirmed by the surrounding Acta2⁺ SMC. Venular endothelium was stained with Vwf but not adjacent to SMC. CapECs were negative for Vwf staining. Three dimensional quantifications of EC subtypes have revealed that the loss of brain ECs in APP/PS1 mice is due to the selective loss of vECs and capECs.

### Example 3: RIPK1 Dependent Endothelial Loss in AD Brain

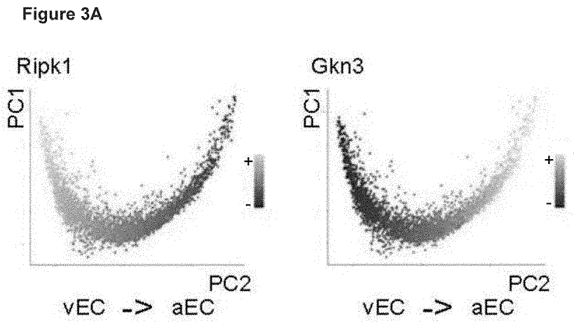

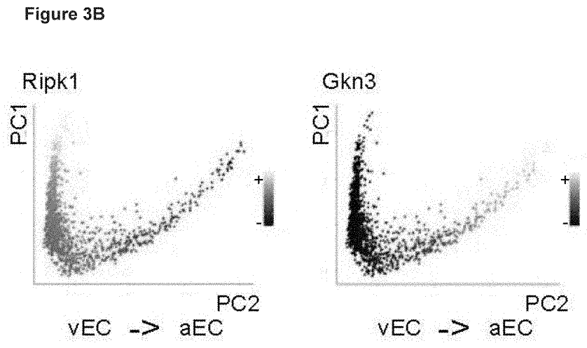

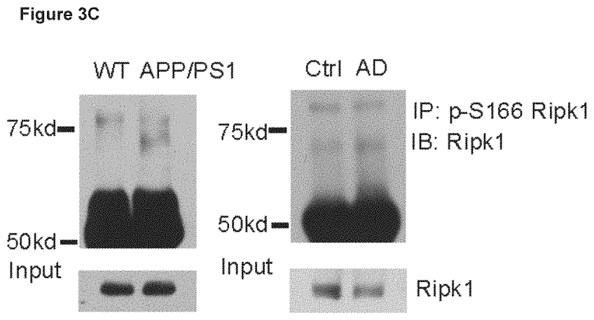

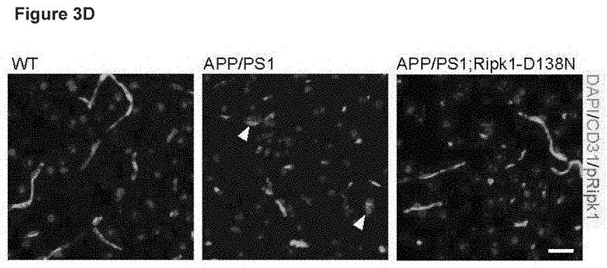

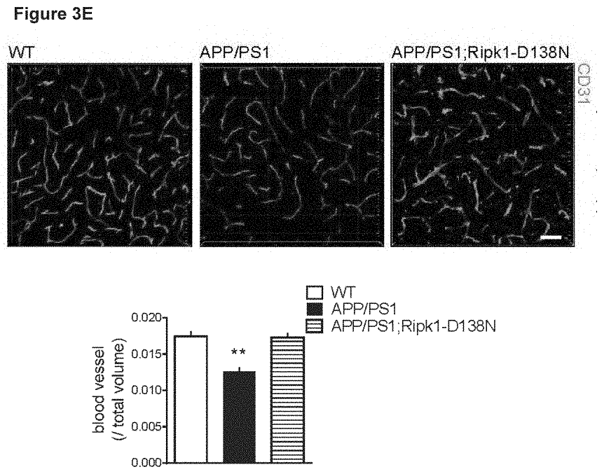

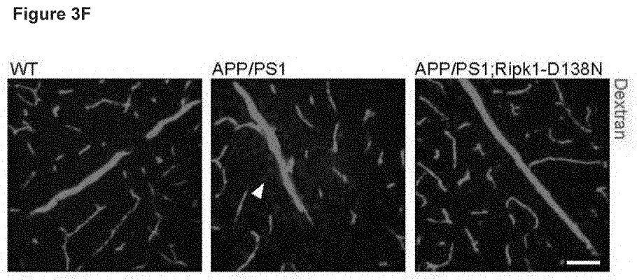

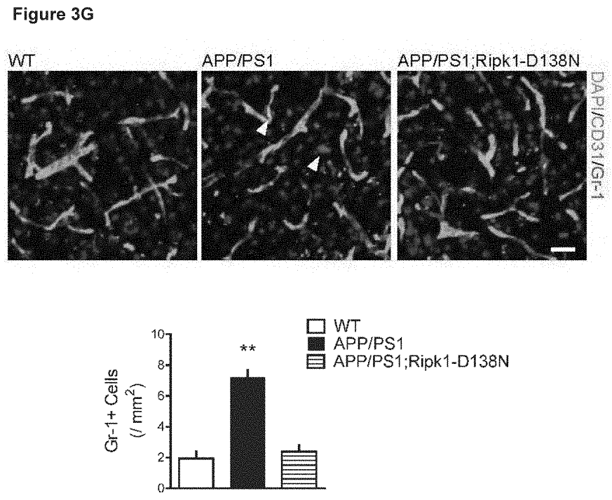

To unravel the molecular basis of selective loss on vECs and capECs in the pathogenesis of AD, the expressions of signature genes that regulates cell death were plotted along endothelial venous to arterial axis at single cell resolution. RIPK1, instead of RIPK3, Mlk1, TNF receptor, Caspase 3, Caspase 8 and Caspase 9, is found to be expressed at a higher level in vECs and capECs compared with aECs, which is further confirmed using publicly available data of cerebral endothelial cells (Vanlandewijck, M. et al. Nature 554, 475-580 (2018)) (FIGS. 3A and 3B). Recent reports have also demonstrated the propensity of peripheral ECs to die vian RIPK1 dependent pathways (Zelic, M. J. Clin. Invest. 128(5): 2064-2075 (2018)). As RIPK1's deleterious effects on cell death are predominantly mediated by its kinase activity assessed by its auto-phosphorylation at S166 (Degterev, A. et al. Nat. Chem. Biol. 4(5): 313-321 (2008)), microvessels were isolated from mouse brains or human brains (Munikoti, V. V., et al. J. Neurosci Methods 207(1): 80-85 (2012); Boulay, A. C., et al. J. Vis. Exp. 105, e53208 (2015)) and immunoprecipitation was performed against p-S166 RIPK1. Western blotting of RIPK1 has revealed an increase of RIPK1 activation in brain microvessels of APP/PS1 mice or AD patients (FIG. 3C). The colocalization of p-S166 RIPK1 staining with CD31 in the sections of APP/PS1 mouse brain has further confirmed RIPK1 is activated in brain microvascular ECs during AD pathogenesis (FIG. 3D). Genetic inactivation of RIPK1 activity with a kinase-dead D138N mutation could rescue AD phenotypes in APP/PS1 mice (APP/PS1; D138N) (Ofengeim, D., et al. Proc. Nat1. Acad. Sci. U.S.A. 114(41), E8788-E8797 (2017)). Such a transgenic mouse model was used in this Example to find a rescue of ECs loss in AD mouse brain by RIPK1 activity inhibition (FIG. 3E). Importantly, permeability of the blood-brain-barrier (BBB) as measured by the leakage of Dextan and neutrophil migration into cerebral parenchyma has also been protected by the inhibition of RIPK1 activity during AD pathogenesis (FIGS. 3F and 3G).

### Example 4: RIPK3 Dependent Endothelial Necroptosis in AD Brain

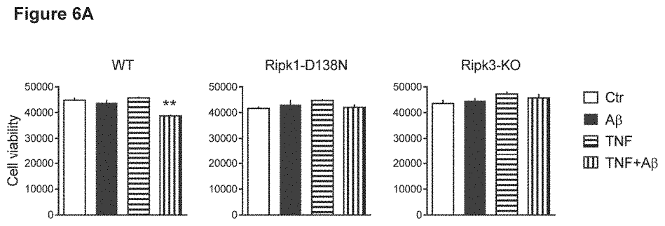

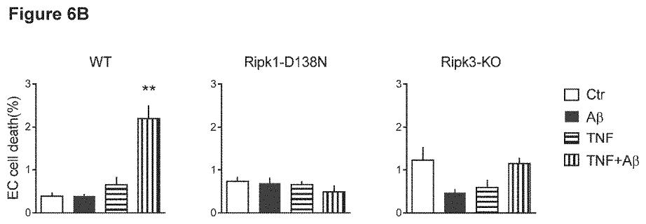

To pin down the mechanism that causes RIPK1 dependent endothelial loss in AD brain, ex vivo cultured primary ECs from mouse brains (Welser-Alves, J. V., et al. Methods Mol. Biol. 1135: 345-356 (2014)) were used. In the presence of TNFα and oligomeric Aβ1-42, inhibition of RIPK1 kinase activity and knockout of RIPK3 have exhibited equivalent levels of resistance to cell death (FIGS. 6A and 6B), which indicates that ECs are likely dying via necroptosis in AD development (Korbelin, J., et al. EMBO Mol. Med. 8(6): 609-625 (2016)). Therefore selectively knockdown of RIPK3 in brain ECs may be a viable strategy to rescue EC loss during AD pathogenesis. An AAV vector that highly selectively transduces brain microvascular ECs (e.g., as described in Korbelin et al. EMBO Mol Med 8(6): 609-25 (2016)) was used to deliver RIPK3 shRNA.

An exemplary method for production RIPK3 shRNA virus particles is as follows. AAV vector plasmids carrying a vector genome with RIPK3 shRNA (CCTCAGATTCCACATACTTTA) or control shRNA (CAACAAGATGAAGAGCACCAA) under the control of H1 promoter were transfected into, e.g., HEK293 cells with AAV2-BR1 packaging plasmid and adenovirus plasmid. Additional information about the design and/or production of RIPK3 shRNA virus particles can be found in Korbelin et al. EMBO Molecular Medicine 8(6): 609-625 (2016), the contents of which are incorporated herein by reference in their entireties. The recombinant viruses were purified, e.g., by standard CsCl gradient sedimentation method and desalted by dialysis, e.g., as described in Mueller et al., Current protocols in microbiology. Chapter 14 (2012), the contents of which are incorporated herein by reference in their entireties.

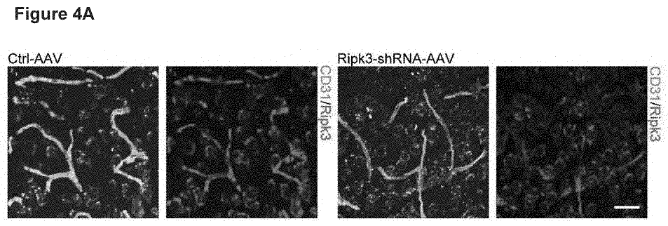

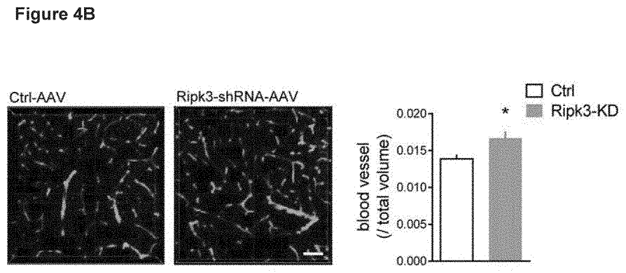

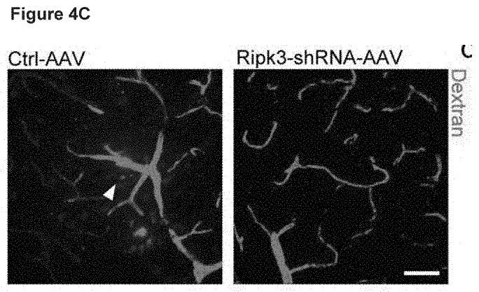

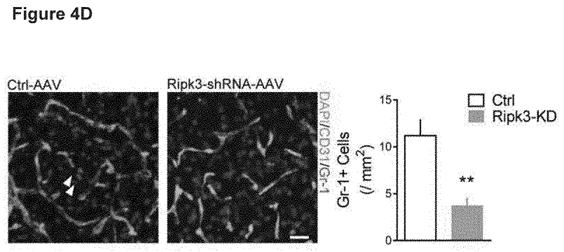

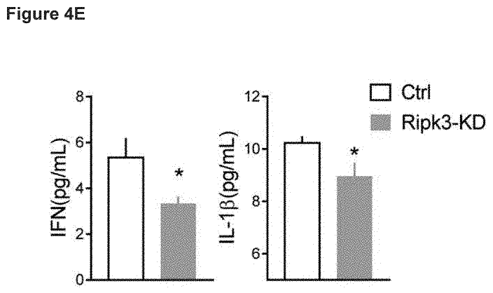

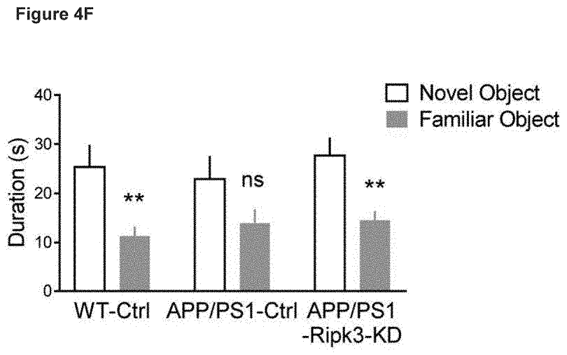

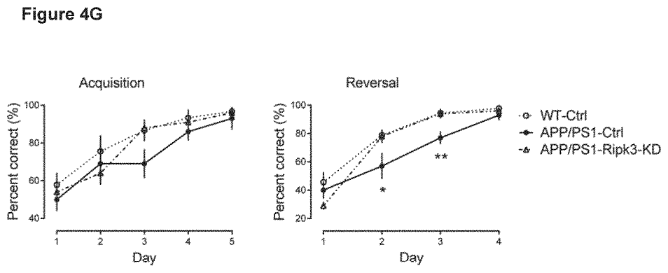

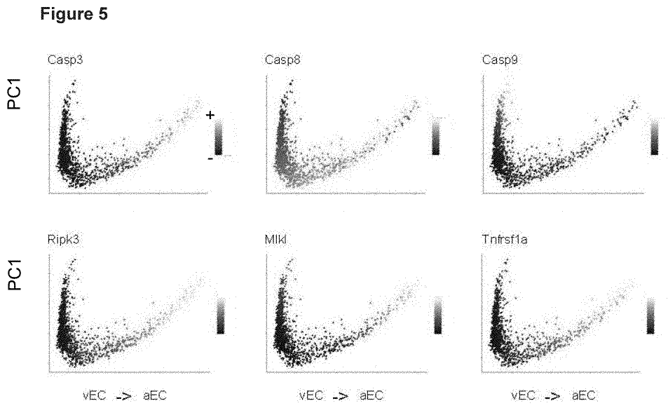

RIPK3 protein expression level in brain microvascular ECs of APP/PS1 mice was greatly reduced after injecting the RIPK3-shRNA-AAV. RIPK3 knockdown (RIPK3-KD) has successfully rescued endothelial loss and the increase of BBB permeability in APP/PS1 mice (FIG. 4B-D), confirming ECs undergo necroptosis in the brain of AD mice during AD progression. Blocking brain endothelial necroptosis and BBB leakage by RIPK3-KD also has ameliorated neuroinflammation in AD brain (FIG. 4E). More importantly, RIPK3-KD in brain ECs has been able to rescue cognitive deficits in APP/PS1 mice as examined by both novel object recognition test and water T maze test (FIGS. 4F and 4G).

### Example 5: Nat1 Expression is Reduced in AD

During Alzheimer's disease (AD) pathogenesis, dysfunction of cerebral endothelial cells (ECs) progresses over the course of the disease (Sweeney et al. Nature reviews 14: 133-150 (2018). There is an increasing appreciation of the contribution from brain vascular malfunction to the initiation of AD clinical symptoms (Iadecola Neuron 80: 844-866 (2013); Launer et al. Neurology 52: 78-84 (1999); and Di Marco et al. Neurobiology of disease 82: 593-606 (2015)). Dementia with both vascular and neurodegenerative components has emerged as a leading cause of age-related cognitive impairment. The mechanisms that mediate EC death and dysfunction in AD development still remain understudied. Also, it is still unclear whether EC dysregulation in AD brain is the primary driver of cognitive decline, and whether preventing the dysfunction of EC is a viable therapeutic strategy for AD. Furthermore, type II diabetes has been linked with increased incidence of AD. However, the mechanism by which diabetes promotes AD is unclear.

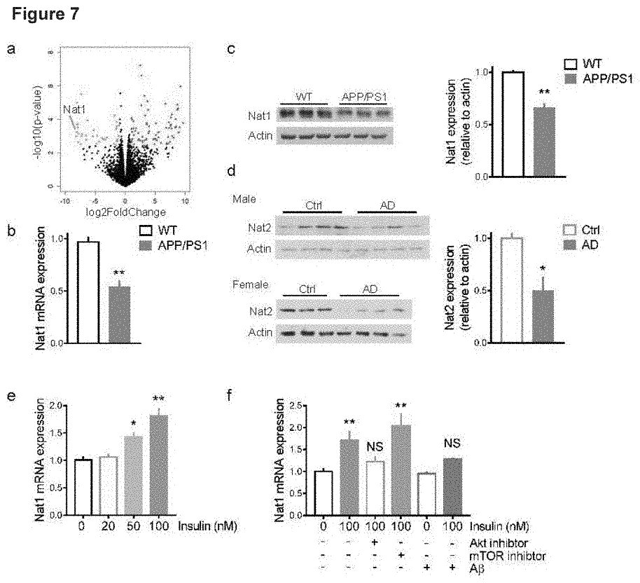

To understand the mechanisms that mediate cerebral EC death and dysfunction in the pathogenesis of AD, fluorescence-activated cell sorting (FACS) was used to isolate ECs from the cerebrum of wild-type (WT) and APPswePSEN1dE9 (APP/PS1) mice (5-6 months of age). RNA sequencing (RNAseq) was used to profile gene expression between these groups, and differential gene expression was analyzed using edgeR (as described in Robinson, M. D., et al. Bioinformatics 26, 139-140 (2010)) (FIG. 7A). Between the WT and APP/PS1 groups, 116 genes were differentially expressed in cerebral ECs. Surprisingly, of these, the most significantly altered expression was found in Nat1, which functions as a protector of necroptosis in our previous screening database. The decrease in Nat1 mRNA expression in cerebral ECs during AD pathogenesis was further confirmed by quantitative PCR (qPCR) (FIG. 7B). At the protein level, it was found that NAT1 was also downregulated in the brains of microvessels isolated from APP/PS1 mice (FIG. 7C). To validate the relevance of this finding in human AD pathogenesis, protein levels of NAT2 (the human homolog of mouse NAT1) in microvessels isolated from the brains of AD patients were assessed and compared to age-matched controls. It was found that there was also a loss of protein in AD patients (FIG. 7D). Previous reports have shown that Nat1 knockout (Nat1 KO) mice display global insulin resistance, and that NAT2 in humans was an insulin-responsive gene, although the mechanism by which this occurred is not known (Knowles, J. W. et al. The Journal of clinical investigation 125, 1739-1751 (2015); Camporez, J. P. et al. Proceedings of the National Academy of Sciences of the United States of America 114, E11285-E11292 (2017)). To determine whether Nat1 expression might also be insulin-sensitive in immortalized mouse cerebral ECs (bEnd.3 cells), it was assessed whether increased concentrations of insulin could upregulate Nat1 (FIG. 7E). Interestingly, amyloid beta (A(3) has been shown to competitively bind to the insulin receptor, identifying a possible mechanism by which type 2 diabetes increases the risk of developing AD (Xie, L. et al. The Journal of neuroscience: the official journal of the Society for Neuroscience 22, RC221 (2002)). By pre-treating bEnd.3 cells with oligomeric Aβ1-42, insulin-induced upregulation of Nat1 mRNA was blocked (FIG. 7F). Similarly, by directly inhibiting insulin signaling via the Akt pathway using the inhibitor MK-2206, but not mTOR signaling using rapamycin, the impact of insulin on Nat1 expression was negated.

### Example 6: Nat1 Knockdown Sensitizes Endothelial Cells to Cell Death

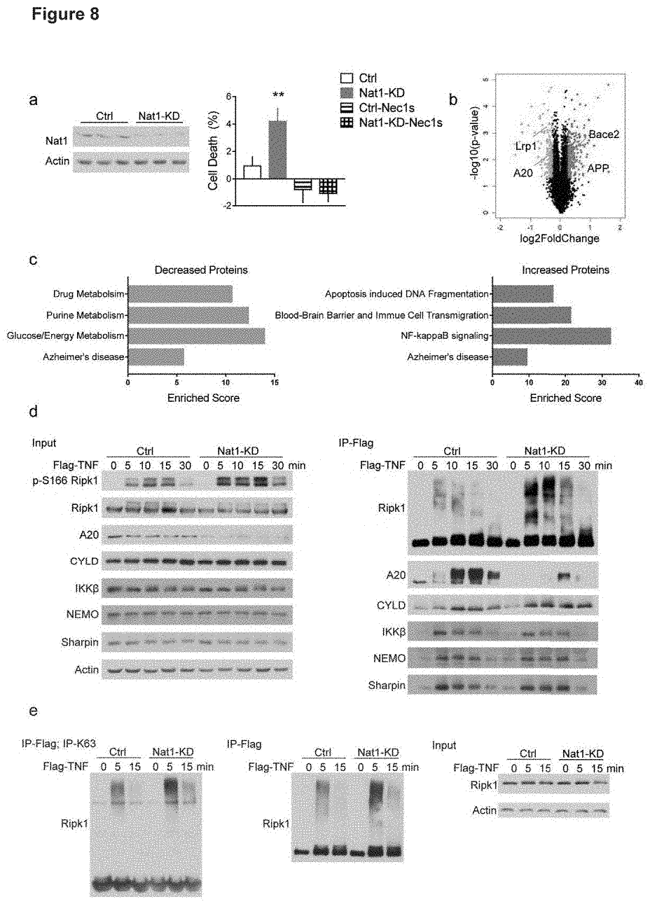

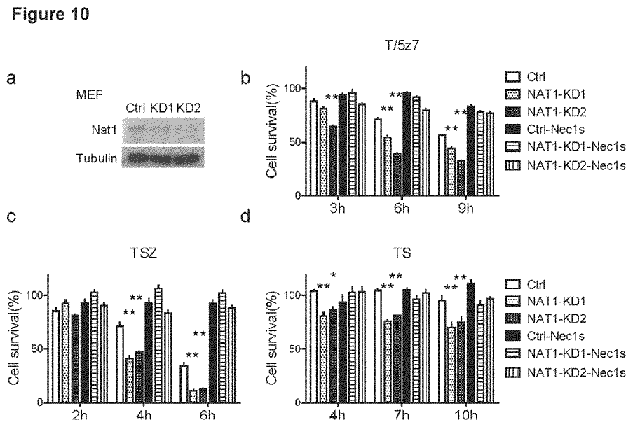

To better understand the role of Nat1 in AD pathogenesis, mouse cerebral ECs (bEnd.3) were transfected with Nat1-KD-shRNA (Nat1-KD) or control shRNA (Ctrl). The knockdown effect was confirmed by Western Blot (FIG. 8A). Cells with Nat1-KD treated with TNFα were significantly more prone to cell death (FIG. 8A). By inhibiting RIPK1 kinase activity with Nec-1s, the elevated cell death was rescued (FIG. 8A). Interestingly, it was found that Nat1-KD sensitized to multiple forms of RIPK1-regulated, TNFα-dependent cell death, including necroptosis and RIPK1-dependent apoptosis (RDA) (FIG. 10). This indicates that Nat1 may be acting as an upstream regulator sensitizing to cell death. To determine how Nat1-KD might be altering cell death pathways, a global quantitative analysis of the proteome was conducted using Tandem Mass Tag isobaric labeling mass spectrometry (TMT-MS). It was found that 7,707 proteins were altered between Nat1-KD and Ctrl bEnd.3 cells, 265 of which were upregulated in Nat1-KD cells (FIG. 8B). Of great interest, multiple proteins implicated in AD, e.g. amyloid precursor protein (APP) and β-secretase 2 (BACE2) are both increased in Nat1-KD cells, indicating a potential upregulation of the Aβ-driven feedback loop of Nat1 downregulation. In the proteome, 181 proteins were downregulated in Nat1-KD cells, which included low density lipoprotein receptor-related protein 1 (LRP1), an essential Aβ transporter from the brain to the periphery, and tumor necrosis factor alpha-induced protein 3 (A20, encoded by Tnfaip3) (FIG. 8B). In line with the role of Nat1 in metabolism of drugs and other non-endogenous substrates, gene set enrichment analysis (GSEA) identified drug, purine, and glucose/energy metabolism as the most highly downregulated pathways in the proteome (FIG. 8B). Strikingly, Nat1-KD showed a significant upregulation of proteins in the NF-κB, BBB trafficking and immune cell migration, and apoptosis-induced DNA fragmentation pathways, supporting the role that Nat1 plays in sensitizing to cell death and BBB damage (FIG. 8C). Knocking down Nat1 also altered AD related protein expression in both directions, validating that it plays an important role in mediating AD progression (FIG. 8C).

Since A20 is an important suppressor of RIPK1 activation, RIPK1 activation and Complex I assembly were next assessed in Nat1-KD or Ctrl bEnd.3 cells treated with TNFα. Nat1-KD sensitizes to multiple forms of RIPK1-regulated cell death. These cells showed greatly increased levels of RIPK1 activation as measured by p-S166, and increased ubiquitination in Complex I (FIG. 8D). A20 levels were also significantly reduced in Nat1-KD total lysates by western blotting, and even further in Complex I, indicating an impairment in A20 recruitment to the TNFRSC (FIG. 8D). While total levels of RIPK1 do not appear to be altered in Nat1-KD cells relative to Ctrl bEnd.3, there is a significant increase in K63 ubiquitination of RIPK1 in Complex I (FIG. 8E).

### Example 7: A20 is Downregulated in the Absence of Nat1

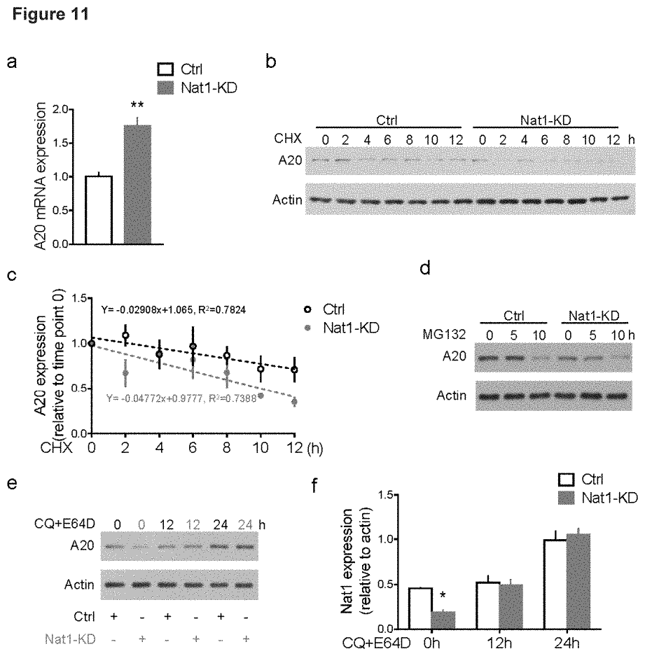

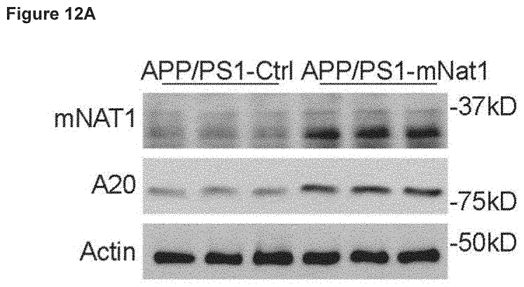

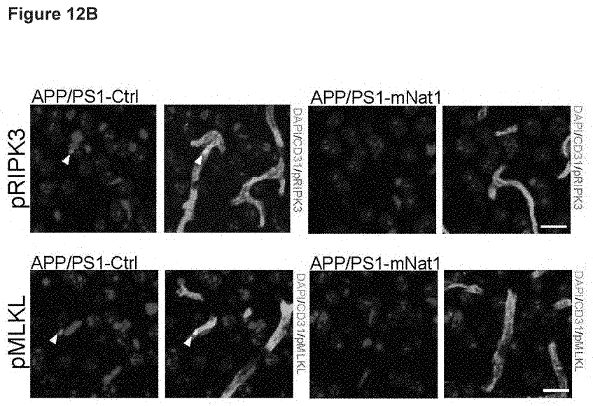

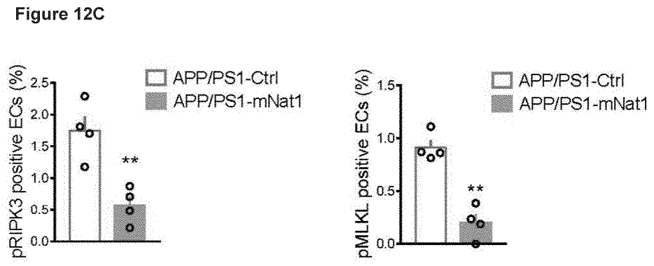

To understand the mechanism by which A20 is decreased in Nat1-KD cells, it was first assessed whether this alteration was occurring at the protein or mRNA level. Unexpectedly, in Nat1-KD cells, mRNA levels of Tnfaip3 are increased relative to Ctrl bEnd.3 cells, indicating that alteration of A20 is occurring at the protein level (FIG. 12A). Nat1-KD and Ctrl bEnd.3 cells were treated with cycloheximide to investigate the kinetics of A20 degradation. While the half-life of A20 in Ctrl cells was 19.4 hours, knocking down Nat1 shortened the half-life to 10.0 hours (FIGS. 12B and 12 C). It was found that inhibition of proteasomal degradation with MG132 reduced protein expression of A20 in both Ctrl and Nat1-KD cells, indicating that proteasomal degradation is not responsible for the changes seen in A20 protein level between these conditions (FIG. 12D). It was next assessed whether the pathway of A20 trafficking to lysosomes (Li et al. Biochimica et biophysica acta 1793, 346-353 (2009)) may be altered in Nat1-KD cells. As shown in FIGS. 12E and 12F, inhibition of lysosomal protein degradation by chloroquine and E64D rescued A20 protein levels in Nat1-KD cells to levels seen in Ctrl bEnd.3 cells.

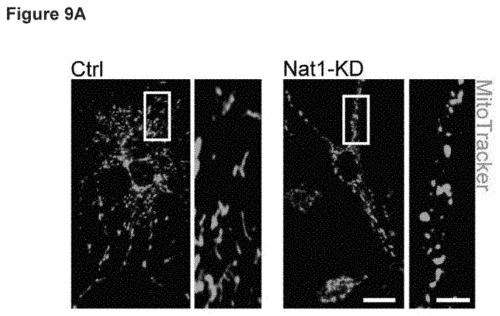

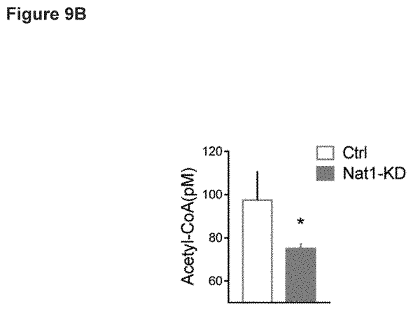

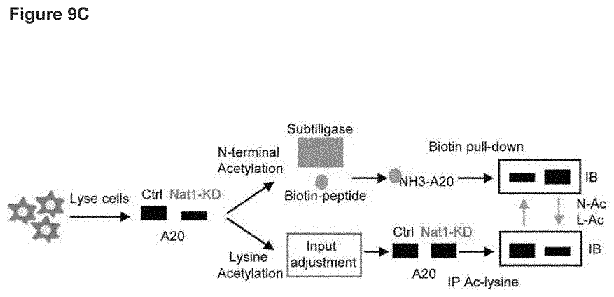

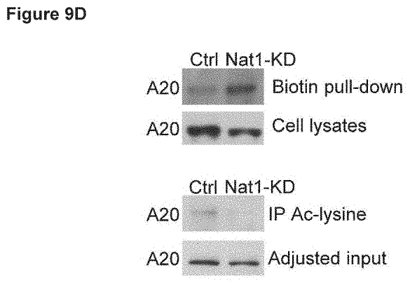

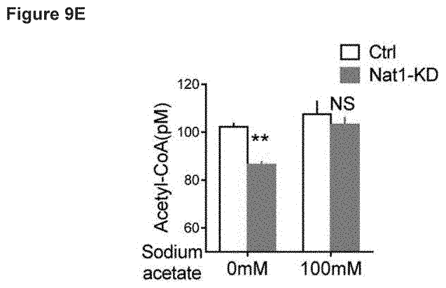

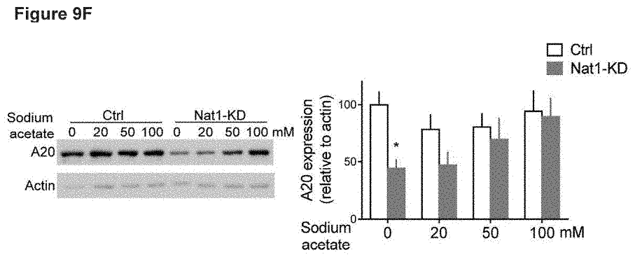

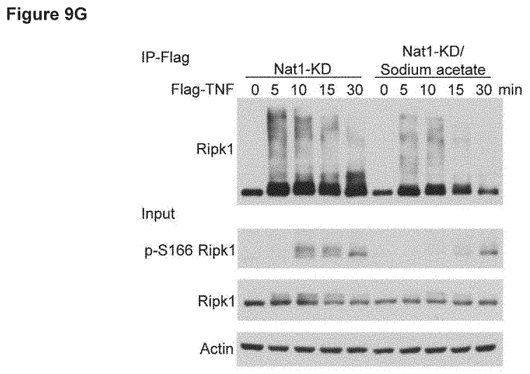

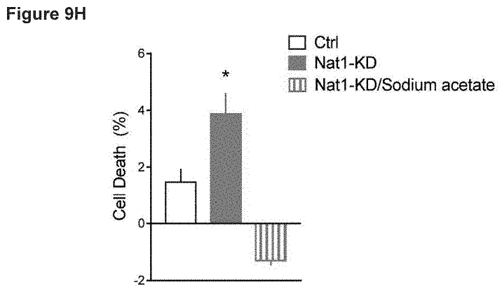

Analysis of Nat1 knockout mice previously reported increased mitochondrial damage and fragmentation (Camporez, J. P. et al. Proceedings of the National Academy of Sciences of the United States of America 114, E11285-E11292 (2017)). As shown in FIG. 9A, MitoTracker live imaging showed damaged mitochondria in Nat1-KD bEnd.3 cells. Since Nat1 deficiency affects metabolism which may affect the cellular levels of acetyl-CoA, the concentration of total acetyl-CoA in Nat1-KD cells was determined. It was found that the levels of acetyl-CoA were reduced in Nat1-KD cells (FIG. 9B). Since protein N-terminal acetylation has been shown to play an important role in regulating cell death (Yi, C. H. et al. Cell 146, 607-620 (2011), it was next assessed whether Nat1 might regulate the cellular sensitivity to cell death by affecting N-terminal acetylation of proteins. To examine the involvement of N-terminal acetylation in regulating the levels of A20, N-terminal acetylation and lysine acetylation of A20 were compared between Nat1-KD and Ctrl bEnd.3 cells (FIG. 9E). The subtiligase reaction identified a decrease in N-terminal acetylation of A20, as assessed by biotin pulldown; similarly, lysine acetylation of A20 is also reduced in Nat1-KD cells (FIG. 9D). The addition of sodium acetate to culture media, which stimulates cytosolic acetyl-CoA production by acetyl-CoA synthetase, was able to rescue decreased acetyl-CoA and A20 in Nat1-KD cells (FIGS. 9E and 9F). Further, supplementation with sodium acetate is also able to rescue the hyperactivation of RIPK1 in Nat1-KD cells in response to TNFα, as measured by RIPK1 ubiquitination (FIG. 9G). This reduction in RIPK1 ubiquitination corresponds to a rescue of the sensitization to cell death otherwise seen in Nat1-KD cells (FIG. 9H).

### Example 8: Overexpression of Nat1 can Rescue Cognitive Decline in AD

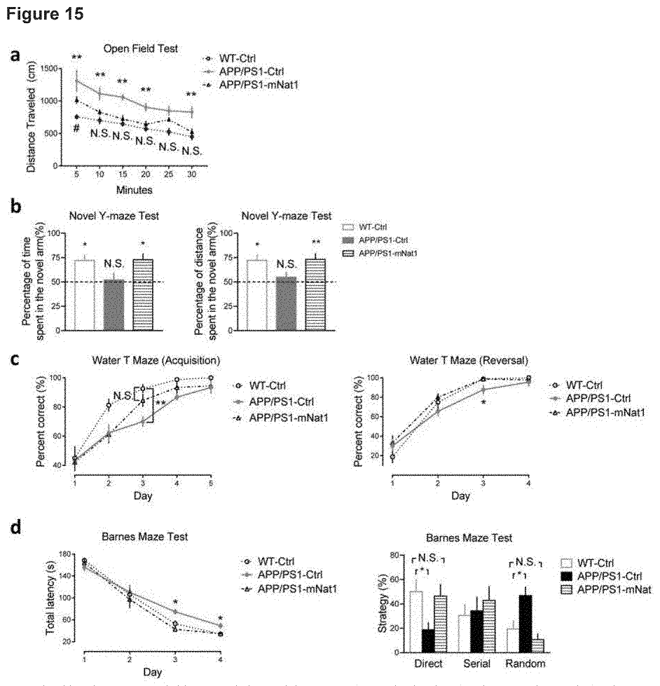

In this Example, it was assessed whether overexpression of Nat1 in cerebral ECs may rescue behavioral deficits seen in APP/PS1 mice during AD pathogenesis. Using a cerebral EC specific AAV, upregulation of Nat1 in cerebral ECs rescued deficits in spatial memory as measured by the Water T Maze in APP/PS1 mice relative to WT littermates (FIG. 15C).

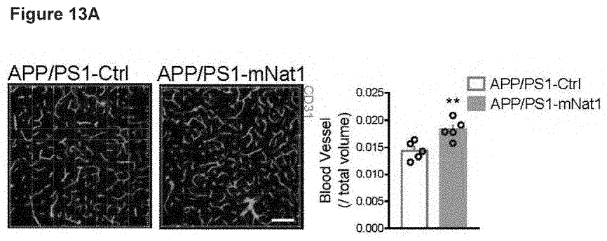

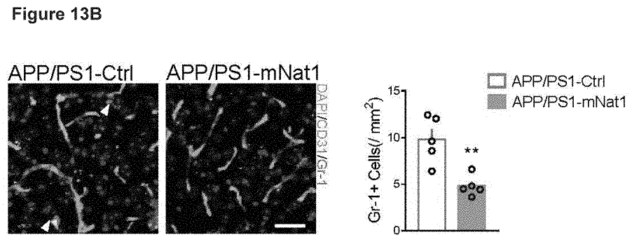

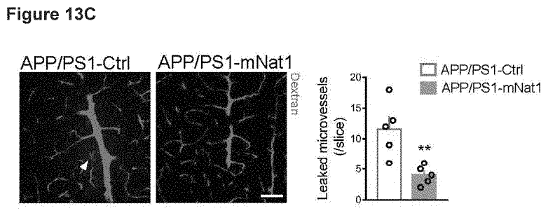

It was found that by regulating cellular metabolism, Nat1 deficiency leads to reduction in the levels of acetyl-CoA which in turn changes the cellular protein stability in cerebral endothelial cells. Competitive binding of Aβ to the insulin receptor of cerebral endothelial cells, which subsequently downregulates Nat1. Downregulation of Nat1 decreases acetylation and the protein expression of A20, a critical regulator of RIPK1 activation. Decreased expression of A20 via Nat1-KD results in RIPK1 activation and sensitization to cell death in cerebral ECs. Loss of Nat1 promotes BBB damage, which mirrors that seen in the APP/PS1 mouse model of AD. Interestingly, the overexpression of NAT1 in a highly targeted manner in cerebral ECs is able to rescue behavioral deficits seen in APP/PS1 mice (FIG. 15). Overexpression of NAT1 leads to increased A20 expression, reduced levels of necroptosis markers p-RIPK3 and p-MLKL, and restored cerebral microvessels in AAP/PS1 mice (FIGS. 12A-12C and FIG. 13A-13C).Taken together, data presented herein provide a mechanism linking insulin resistance and Aβ to neuroinflammation and cell death, indicating Nat1 as a novel target regulating cerebrovascular and metabolic health in AD.

### Example 9: LRP1 is Downregulated in AD and Restoration of Nat1 can Promote LRP1-Mediated Clearance of Amyloid Beta

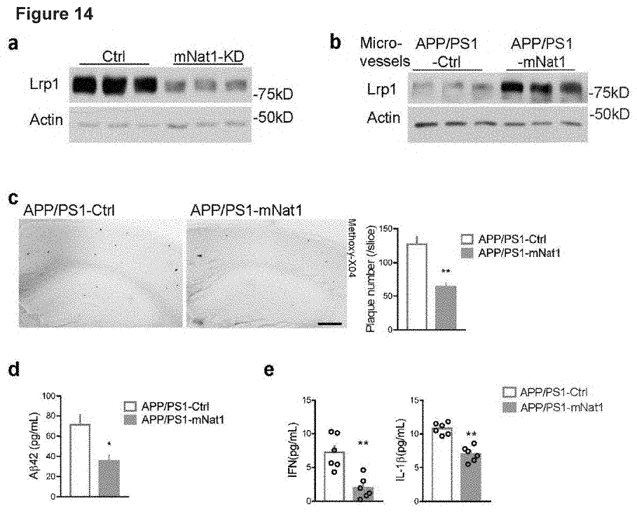

In this Example, it was assessed whether overexpression of Nat1 in cerebral ECs may increase Lrp1 protein expression. Lrp1 levels were compared in control and Nat1-KD endothelial cells using Western Blot, demonstrating that lower levels of Lrp1 are produced with decreased Nat1 expression (FIG. 14, panel A). Using a cerebral EC specific AAV, upregulation of Nat1 in cerebral ECs leads to increased Lrp1 expression in the microvessels of APP/PS1 mice (FIG. 14, panel b). Overexpression of Nat1 also leads to reduced levels of Aβ (FIG. 14, panels c and d) and inflammatory cytokines IFN and IL-1(3 in brains of APP/PS1 mice (FIG. 14, panel e). Taken together, data presented herein provide a mechanism for Nat1 regulation of Aβ formation and cytokine IFN and IL-1β levels through changes in Lrp1 protein expression.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A method of treating a neurovascular disease or disorder in a subject, the method comprising inhibiting necroptosis in brain endothelial cells of the subject.

2. The method of claim 1, wherein the step of inhibiting necroptosis comprises performing at least one of the following steps:
a. inhibiting expression and/or activity of receptor-interacting serine/threonine-protein kinase 3 (RIPK3) in the brain endothelial cells of the subject;
b. inhibiting expression and/or activity of receptor-interacting serine/threonine-protein kinase 1 (RIPK1) in the brain endothelial cells of the subject;
c. increasing expression and/or activity of N-acetyltransferase 1 (Nat1) or N-acetyltransferase 2 (Nat2) in the brain endothelial cells of the subject;
d. increasing expression and/or activity of tumor necrosis factor alpha-induced protein 3 (Tnfaip3/A20) in the brain endothelial cells of the subject; and
e. increasing expression and/or activity of low density lipoprotein receptor-related protein 1 (LRP1) in the brain endothelial cells of the subject.

3. The method of claim 1, wherein:
a. the step of inhibiting necroptosis comprises a step of inhibiting expression and/or activity of RIPK3 in the brain endothelial cells of the subject; and
b. the step of inhibiting expression and/or activity of RIPK3 comprises administering to the subject a brain endothelial-cell-specific viral vector that expresses a nucleic acid agent that inhibits expression of RIPK3, wherein the nucleic acid reduces RIPK3 transcript level, RIPK3 level, or both, relative to comparable conditions when the nucleic acid agent is absent.

4. The method of claim 3, wherein the nucleic acid agent is or comprises an RNA agent whose nucleotide sequence is sufficiently complementary to that of an RIPK3 transcript, or portion thereof, that the nucleic acid agent hybridizes to the RIPK3 transcript.

5. (canceled)

6. The method of claim 3, wherein the nucleic acid agent is or comprises a short hairpin RNA (shRNA).

7. The method of claim 3, wherein the viral vector is an adeno-associated viral vector.

8. The method of claim 1, wherein:
a. the step of inhibiting necroptosis comprises a step of increasing expression and/or activity of N-acetyltransferase 1 (Nat1) or N-acetyltransferase 2 (Nat2) in the brain endothelial cells of the subject; and
b. the step of increasing expression and/or activity of Nat1 or Nat2 comprises administering to the subject a brain endothelial-cell-specific viral vector comprising a nucleic acid agent that encodes Nat1 or Nat2, or a functional fragment thereof, wherein the nucleic acid increases Nat1 or Nat2 transcript level, Nat1 or Nat2 level, or both relative to comparable conditions when the nucleic acid agent is absent.

9. (canceled)

10. The method of claim 8, wherein the viral vector is an adeno-associated viral vector

11. The method of claim 8, wherein, when the subject is a mouse subject, the step of inhibiting necroptosis comprises a step of increasing expression of Nat1.

12. The method of claim 8, wherein, when the subject is a human subject, the step of inhibiting necroptosis comprises a step of increasing expression of Nat2.

13. The method of claim 1, wherein the neurovascular disease or disorder is Alzheimer's disease.

14. The method of claim 1, wherein the brain endothelial cells comprise brain microvessels or microvasculature; are cerebral endothelial cells, or blood-brain barrier endothelial cells, or any combination thereof.

15. The method of claim 1, wherein:
a. the subject is suffering from or susceptible to neuroinflammation in the subject's brain; and
b. the step of inhibiting comprises inhibiting to an extent sufficient that the neuroinflammation is reduced.

16. The method of claim 15, wherein level of the neuroinflammation is assessed by detecting level of interferon or interleukin-1β or both.

17. A method of inhibiting necroptosis in the brain of a subject in need thereof, the method comprising increasing expression and/or activity of N-acetyltransferase 1 (Nat1) or N-acetyltransferase 2 (Nat2) in the brain of the subject, wherein the step of increasing comprises administering an agonist of Nat1 or Nat2 to the subject.

18. The method of claim 17, wherein the step of increasing comprises increasing to an extent sufficient to inhibit activation of (RIPK1) activation or to increase the level of A20 in the brain of the subject.

19.-21. (canceled)

22. The method of claim 17, wherein the agonist is or comprises an expression vector comprising a nucleic acid sequence that encodes Nat1 or Nat2, or a functional fragment thereof.

23. (canceled)

24. The method of claim 22, wherein the expression vector is an adeno-associated viral vector or a cerebral endothelial-cell-specific viral vector.

25. (canceled)

26. The method of claim 17, wherein the subject is suffering from or susceptible to a necroptosis-mediated neurovascular disease or disorder.

27. The method of claim 26, wherein the necroptosis-mediated neurovascular disease or disorder is Alzheimer's disease.
